(19) United States
(12) Patent Application Publication
(10) Pub. No.: US 2022/0056457 A1
(43) Pub. Date: Feb. 24, 2022

(54) CIS CONJUGATIVE PLASMID SYSTEM

(71) Applicant: THE UNIVERSITY OF WESTERN ONTARIO, London (CA)

(72) Inventors: David R. EDGELL, London (CA); Gregory B. GLOOR, London (CA); Bogumil J. KARAS, London (CA)

(73) Assignee: THE UNIVERSITY OF WESTERN ONTARIO, London, ON (CA)

(21) Appl. No.: 17/413,214

(22) PCT Filed: Dec. 11, 2019
(86) PCT No.: PCT/CA2019/051787

§ 371 Date: Jun. 11, 2021

Related U.S. Application Data
(60) Provisional application No. 62/777,869, filed on Dec. 11, 2018.

(51) Int. Cl. C12N 15/70; C12N 15/11; C12N 9/22; C12Q 1/689

(52) U.S. Cl. CPC C12N 15/70; C12N 15/11; C12N 9/22; C12N 2800/101; C12N 2310/20; C12N 2800/80; C12Q 1/689

(57) ABSTRACT

A method for modulating a target organism in a microbiome, comprising contacting the microbiome with a cis-conjugative plasmid that can replicate and conjugate with organisms in the microbiome including the target organism, the conjugative plasmid comprising conjugation genes and a gene or a combination of genes capable of being expressed in the target organism and that only modulates the target organism in the microbiome. Also the isolated cis-conjugative plasmid comprising conjugation genes and a gene or a combination of genes capable of being expressed in a target bacteria within a microbiome or biofilm and that modulates the target bacteria in the microbiome or biofilm.

### FIELD OF THE INVENTION

The present invention relates in general to plasmid systems, more particularly to cis conjugative plasmid systems and methods of using cis conjugative plasmid systems for altering a microbiome or biofilm or detecting constituents of a microbiome or biofilm.

### BACKGROUND OF THE INVENTION

Microbial ecosystems are essential for human health and proper development, and disturbances of the ecosystem correlate with a multitude of diseases [1-5]. A central problem is the lack of tools to selectively control pathogenic species that cause disease, or to otherwise alter or transform the composition of the human or non-human microbiome.

Microbes persisting in a biofilm in the human body cause about two-thirds of all chronic/recurrent diseases. These biofilms are composed of bacteria and other microbes protected by an extracellular matrix that is often made up of polysaccharides, proteins and DNA which prevents the innate and adaptive immune systems, antibiotics, bacteriophage and other antibacterial agents from gaining access to the bacteria inside the biofilm. Biofilms protect the microbes by forming a barrier and make it extremely difficult to clear the infection from the body. Furthermore, biofilms can act as a reservoir for future acute infections often with lethal consequences.

Traditional methods to modify microbial communities suffer from a number of disadvantages or limitations.

Antibiotic treatment suffer from a number of limitations that preclude selective control in a defined and efficient manner, and are becoming less effective because of overuse and the development of multi-drug resistant bacteria.

Phage-based therapy is limited by host range and the rapid development of phage-resistant bacteria [6].

Probiotics and prebiotics are effective but of use in only a few defined conditions [⁷].

Stool transplants are effective treatments for gastrointestinal dysbioses, but can result in wide-spread alterations in the composition of the microbial ecosystem with unknown long-term effects [8-10].

The limitations of the traditional methods highlight an increasing need for effective and selective tools for the targeted modification of microbiomes.

Conjugative plasmids are an attractive tool to alter or modify microbiomes because conjugative plasmids have broad host ranges, are generally tought to be resistant to restriction-modification systems, are easy to engineer with large coding capacities, and do not require a cellular receptor that would provide a facile mechanism for bacterial resistance.

A low efficiency of conjugation was found to be a limiting factor in the use of trans-conjugative plasmids.

In view of the foregoing, a new tool to modify microbiomes efficiently and without including the limitations of the prior art is needed.

### SUMMARY OF THE INVENTION

Provided herein is a new cis-conjugative plasmid system and method of using said cis conjugative plasmid system in altering a bacterial microbiome or biofilm. The cis-conjugative plasmid encodes both the conjugative machinery and a gene or combination of genes of interest to alter or modify or modulate target bacteria species in the bacterial microbiome or biofilm, as opposed to previously tested trans setups where the conjugation machinery and gene of interest were separated (FIG. 1). Any bacterium in the bacterial microbiome or the biofilm that receives the cis-conjugative plasmid of the present invention becomes a potential donor for subsequent rounds of re-conjugation, leading to exponentially increasing numbers of conjugative donor bacteria in a population of bacteria such as a microbiome. The cis-conjugative plasmid of the present invention is highly efficient in conjugative transfer among the different bacteria in the microbiome and can be used to kill, alter, modify or modulate a particular species of bacteria or a particular subpopulation of bacteria within a microbiome or biofilm.

In one embodiment, the present invention is a method for modulating a target organism in a microbiome, comprising contacting the microbiome with a cis-conjugative plasmid that can replicate and conjugate with organisms in the microbiome including the target organism, the cis-conjugative plasmid comprising (i) conjugation genes (i.e. the conjugation machinery) and (ii) a gene or a combination of genes capable of being expressed in the target organism and that modulates the target organism in the microbiome (i.e. gene that modulates the target organism or modulating gene).

In another embodiment, the present invention is a method for modulating a target organism in a microbial biofilm, comprising contacting the microbial biofilm with a cis-conjugative plasmid that can replicate in and conjugate to organisms in the microbial biofilm including the target organism, the cis-conjugative plasmid comprising conjugation genes (i.e. the conjugation machinery) and a gene or a combination of genes capable of being expressed in the target organism and that modulates the target organism in the microbial biofilm (i.e. gene that modulates the target organism or modulating gene).

In another embodiment, the present invention is a method for inhibiting, preventing or treating an infection caused by an organism (“target organism”) that can accept by conjugation and express a conjugative plasmid in a subject, comprising administering to the subject an effective amount of a cis-conjugative comprising conjugation genes (i.e. the conjugation machinery) and a gene or a combination of genes capable of being expressed in the target organism and that modulates the target organism in the microbiome (i.e. gene that modulates the target organism or modulating gene to inhibit, prevent or treat the infection), thereby inhibiting, preventing or treating the infection.

In another embodiment, the present invention is a method for propagating a gene of interest in a target organism within a microbiome or biofilm, comprising contacting the microbiome or biofilm with a cis-conjugative plasmid that can replicate and conjugate organisms in the microbiome or biofilm including the target organism, the cis-conjugative plasmid comprising conjugation genes and a gene or a combination of genes capable of being expressed in the target organism and that modulates the target organism in the microbiome or biofilm to propagate the gene of interest.

In one embodiment of any of the methods of the present invention, the cis-conjugative plasmid further comprises a single or multiple single-guide RNAs corresponding to a single or multiple target sites of the target organism.

In one embodiment of any of the methods of the present invention, the gene that modulates the target organism is a coding region for TevCas9 nuclease gene.

In another embodiment according to any of the methods of the present invention, the gene that modulates the target organism is a coding region for a site-specific DNA endonuclease

In another embodiment according to any of the methods of the present invention, the gene that modulates the target organism is a coding region for a bacterial toxin, wherein the bacterial toxin includes DNA gyrase inhibitors or topoisomerase inhibitors.

In another embodiment according to any of the methods of the present invention, the gene that modulates the target organism is a coding region for a gene or genes for biosynthetic or biodegradative pathways.

In another embodiment according to any of the methods of the present invention, the gene that modulates the target organism is a coding region for regulatory sequence including small RNA molecules or transcription factors.

In another embodiment according to any of the methods of the present invention, the contacting is in vitro or in vivo.

In another embodiment according to any of the methods of the present invention, the target organism is a bacterium.

In another embodiment, the present invention provides an isolated or recombinant cis-conjugative plasmid comprising conjugation genes (i.e. the conjugation machinery) and a gene or a combination of genes capable of being expressed in a target bacteria within a microbiome or biofilm and that modulates the target bacteria in the microbiome or biofilm (i.e. the gene that modulates the target bacteria or modulating gene).

In one embodiment of the isolated or recombinant cis-conjugative plasmid of the present invention, the isolated cis-conjugative plasmid further comprises a single or multiple single-guide RNAs corresponding to a single or multiple target sites of the target bacteria.

In one embodiment of the isolated or recombinant cis-conjugative plasmid of the present invention the gene that modulates the bacteria is a coding region for TevCas9 nuclease gene and guide RNA.

In another embodiment of the isolated or recombinant cis-conjugative plasmid of the present invention the gene that modulates the target bacteria is a coding region for a site-specific DNA endonuclease

In another embodiment of the isolated or recombinant cis-conjugative plasmid of the present invention the gene that modulates the target bacteria is a coding region for a bacterial toxin, wherein the bacterial toxin includes DNA gyrase inhibitors or topoisomerase inhibitors.

In another embodiment of the isolated or recombinant cis-conjugative plasmid of the present invention the gene that modulates the target bacteria is a coding region for a gene or genes for biosynthetic or biodegradative pathways.

In another embodiment of the isolated or recombinant cis-conjugative plasmid of the present invention the gene that modulates the target bacteria is a coding region for regulatory sequence including small RNA molecules or transcription factors.

In another embodiment, the present invention relates to a use of a cis-conjugative plasmid for modulating a target organism in a microbiome or microbial biofilm, the cis-conjugative plasmid being engineered to replicate and conjugate with organisms in the microbiome or microbial biofilm including the target organism, the cis-conjugative plasmid comprising conjugation genes (i.e. the conjugation machinery) and a gene or a combination of genes capable of being expressed in a target bacteria within the microbiome or microbial biofilm and that modulates the target organism in the microbiome or microbial biofilm (i.e. the gene that modulates the target bacteria or modulating gene).

In another embodiment, the present invention relates to a use of a cis-conjugative plasmid for inhibiting, preventing or treating an infection caused by an organism that can accept by conjugation and express a conjugative plasmid in a subject, the cis-conjugative plasmid comprising conjugation genes (i.e. the conjugation machinery) and a gene or a combination of genes capable of being expressed in a target bacteria within the microbiome or microbial biofilm and that modulates the organism that causes the infection to inhibit, prevent or treat the infection, thereby inhibiting, preventing or treating the infection.

In another embodiment, the present invention relates to a use of a cis-conjugative plasmid for propagating a gene of interest in a target organism within a microbiome or biofilm, the cis-conjugative plasmid being capable to replicate and conjugate organisms in the microbiome or biofilm including the target organism, the cis-conjugative plasmid comprising conjugation genes and a gene or a combination of genes capable of being expressed in the target organism and that modulates the target organism in the microbiome or biofilm to propagate the gene of interest.

In one embodiment of the use according to any one of the previous embodiments, the cis-conjugative plasmid further comprises a single or multiple single-guide RNAs corresponding to a single or multiple target sites of the target organism.

In one embodiment of the use according to any of the previous embodiments, the gene that modulates the target organism is a coding region for TevCas9 nuclease gene.

In one embodiment of the use according to any of the previous embodiments, the gene that modulates the target organism is a coding region for a site-specific DNA endonuclease

In one embodiment of the use according to any of the previous embodiments, the gene that modulates the target organism is a coding region for a bacterial toxin, wherein the bacterial toxin includes DNA gyrase inhibitors or topoisomerase inhibitors.

In one embodiment of the use according to any of the previous embodiments, the gene that modulates the target organism is a coding region for a gene or genes for biosynthetic or biodegradative pathways.

In one embodiment of the use according to any of the previous embodiments, the gene that modulates the target organism is a coding region for regulatory sequence including small RNA molecules or transcription factors.

In one embodiment of the use according to any of the previous embodiments, the contacting is in vitro or in vivo.

In one embodiment of the use according to any of the previous embodiments, the target organism is a bacterium.

In another embodiment, the present invention relates to a method of diagnosing an infection caused by a bacteria, the method comprising contacting a site of the infection with a cis-conjugative plasmid comprising conjugation genes (i.e. the conjugation machinery) and a detectable gene specific for the bacteria that causes the infection.

In one embodiment of the method of diagnosing, the detectable gene expresses a detectable protein when the detectable gene is activated by an activator when the activator is in operative proximity to the detectable gene.

In another embodiment of the method of diagnosing, the activator is a transcriptional activation domain.

In another embodiment of the method of diagnosing, the detectable gene is a transposon for transposon-based tagging.

In another embodiment, the present invention is a method of detecting the presence of a bacteria of interest in a microbiome, the method comprising contacting the microbiome with a cis-conjugative plasmid comprising conjugation genes (i.e. the conjugation machinery) and a detectable gene that can only be expressed and active in the bacteria of interest.

In another embodiment, the present invention relates to a kit comprising: (a) an isolated cis-conjugative plasmid comprising conjugation genes and a gene or a combination of genes capable of being expressed in a target organism within a microbiome or biofilm that modulates the target organism in the microbiome or biofilm according to an embodiment of the present invention; and (b) instructions for use in inhibiting, preventing or treating an infection caused by the target organism in the microbiome or biofilm.

In another embodiment, the present invention is an isolated or recombinant nucleic acid sequence comprising SEQ ID NO:66 or an isolated or recombinant nucleic acid sequence having at least 80% sequence identity to SEQ ID NO:66.

In another embodiment, the present invention is an isolated functional fragment of SEQ ID NO:66,

### BRIEF DESCRIPTION OF THE DRAWINGS

The following figures illustrate various aspects and preferred and alternative embodiments of the invention.

FIG. 1: Impact of cis or trans localization of conjugative machinery on conjugation frequency. A. Schematic view of the pNuc-cis and pNuc-trans plasmids. oriT, conjugative origin of transfer; oriV, vegetative plasmid origin; GmR, gentamicin resistance gene; CmR, chloramphenicol resistance gene; TevSpCas9/sgRNA, coding region for TevSpCas9 nuclease gene and sgRNA; Conjugative machinery, genes required for conjugation derived from the IncP RK2 conjugative system. The corresponding nucleotide sequences of each plasmid are provided in Table 3 (SEQ ID NO:66) and Table 4 (SEQ ID NO:67). B. (Top) The TevSpCas9 and sgRNA cassette (not to scale) highlighting the arabinose regulated pBAD and constitutive pTet promoters. (Below) The modular TevSpCas9 protein and DNA binding site. Interactions of the functional TevSpCas9 domains with the corresponding region of substrate are indicated. C. Model of pNuc spread after conjugation with the cis and trans setups. Cell growth overtime will account for increase of pNuc-trans. D. Filter mating assays performed over 24 hr demonstrate that pNuc-cis has a higher conjugation frequency than pNuc-trans. Points represent independent experimental replicates, and the 95\% confidence intervals are indicated as the shaded areas. Conjugation frequency is reported as the number of transconjugants (GmR, KanR) per total recipient S. enterica cells (KanR). E. Conjugation frequency of S. enterica transconjugants harbouring either pNuc-cis or pNuc-trans to naive S. enterica recipients. Data are shown as boxplots with points representing individual replicate experiments F. pNuc-cis and pNuc-trans copy number determined by quantitative PCR in either E. coli or S. enterica. Data are shown as boxplots with solid lines indicating the median of the data, the rectangle the interquartile bounds, and the wiskers the range of the data. Points are individual experiments. G. pNuc-cis and pNuc-trans stability in E. coli or S. enterica determined as the ratio of cells harbouring the plasmid after 24 hrs growth without antibiotic selection over total cells. Data are shown as boxplots with dots indicating independent experiments.

FIG. 2: Optimizing liquid culture conditions for E. coli to S. enterica conjugation. A) Conjugation frequency for different sodium chloride (NaCl) media conditions. B) Conjugation frequency measured with different E. coli donor to S. enterica recipient ratios at the start of conjugation. D) Effect of culture agitation on conjugation frequency (RPM—revolutions per minute). For each plot, points indicate conjugation frequency for independent biological replicates.

FIG. 3: Influence of enhanced cell-to-cell contact on conjugation frequency. A) Schematic of experimental design. Liquid conjugation experiments in culture tubes with B) pNuc-cis and C) pNuc-trans were performed with 0.5 mm glass beads or without glass beads (filled diamonds) over 72 hrs at the indicated shaking speed (in revolutions per minute). Conjugations were performed with (filled circles) or without (filled diamonds) sgRNA targeting the STM1005 locus cloned into pNuc-cis and pNuc-trans. Both plasmids encoded the TevSpCas9 nuclease. Data are plotted on a log 10 as boxplots with data points from independent biological replicates. The solid line represents the median of data, the rectangle represents the interquartile range of the data, and the whiskers represent the maximum and minimum of the data.

FIG. 4: Killing efficiency of sgRNAs targeted to the S. enterica genome. A) Ranked killing efficiency of individual sgRNAs coded as to whether the target site in found in an essential gene (blue filled circles), non-essential gene (orange diamonds), or unknown if the gene is essential (inverted red triangles). Vertical lines represent the standard error of the data from at least 3 biological replicates. B) Killing efficiency of each sgRNA plotted relative to their position in the S. enterica genomes, color-coded as in panel a. The terminator region (ter) and origin of replication (ori) are indicated by vertical red and green lines, respectively.

FIG. 5. Killing of S. enterica by conjugative delivery of TevSaCas9. A) Schematic of TevSaCas9 target site in the fepB gene of S. enterica, with I-Tevl cleavage motif, DNA spacer, sgRNA binding site and PAM motif indicated. B) Plot of S. enterica killing efficiency with no sgRNA cloned in pNuc, or the fepB sgRNA cloned in pNuc. Points are independent biological replicates.

FIG. 6. Killing efficiency of multiplexed pairs of sgRNAs, with single sgRNAs plotted for comparison. Data are plotted on log 10 scale as the mean of at least three independent biological replicates, with vertical lines representing the standard error of the mean. A Mann-Whitney Wilcox test comparing if multiplexed sgRNAs had a significantly higher killing efficiency as a group than their single sgRNA constituents yielded a p-value=0.003.

FIG. 7. Examples of S. enterica escape mutants. A) Nucleotide sequence of the TevSpCas9 target site for STM sgRNA in the Gifsy prophage. Nucleotide substitutions in the seed region of the sgRNA are indicated and underlined. B) Example of an agarose gel of pNuc DNA isolated from EM30 or from wild-type pNuc (+ve) incubated with (+) or without (−) a mixture of FspI and MsII restriction enzymes. Size standards in kilobase pairs (kb) are indicated to the right of the gel image. C) Example of multiplex PCR with pNuc DNA isolated from EM19, EM20 or wild-type pNuc (+ve) with primers specific for the Cm^{R} and TevSpCas9 coding regions.

FIG. 8: Effect of sgRNA targeting parameters on killing efficiency. A) Plot of predicted sgRNA activity versus S. enterica killing efficiency for all 65 sgRNAs. The shaded area is the 95% confidence interval of the line of best fit. Boxplots of sgRNAs targeting different strands for B) transcriptional (S, sense strand; AS, anti-sense strand) and C) replication, and D) sgRNAs targeting genes with essential (Ess), non-essential (NEss) or unresolved phenotypes (Un) versus killing efficiency. E) Plot of relative position of sgRNAs within genes versus average killing efficiency for the sense strand and F) anti-sense strand of targeted genes. For each plot, points are filled according to their predicted sgRNA activity. Killing efficiency is plotted on a log 10 scale.

FIG. 9: Examples of S. enterica escape mutants. A) Nucleotide sequence of the TevSpCas9 target site for STM sgRNA in the Gifsy prophage. Nucleotide substitutions in the seed region of the sgRNA are indicated and underlined. B) Example of an agarose gel of pNuc DNA isolated from EM30 or from wild-type pNuc (+ve) incubated with (+) or without (−) a mixture of FspI and MsII restriction enzymes. Size standards in kilobase pairs (kb) are indicated to the right of the gel image. c) Example of multiplex PCR with pNuc DNA isolated from EM19, EM20 or wild-type pNuc (+ve) with primers specific for the Cm^{R} and TevSpCas9 coding regions.

FIG. 10: Summary of generalized linear model of sgRNA parameters that are indicative of killing efficiency with P-values indicated (left), and a graphical representation of the confidence intervals associated with each parameter. Note that parameters with confidence intervals that pass over the 0 line are not considered significant.

FIG. 11: Example of agarose gel of diagnostic restriction digest of different guideRNAs cloned into pNuc-trans. Each plasmid was digested with EcoRI and KpnI and compared to the pNuc-trans backbone (CTL). Asterisks indicate unexpected digestion patterns. The size of the ladder is indicated in kilo-base pairs (kb) to the left of the gel image.

FIG. 12: Generic representation of the cis-plasmid. Example generic cis-plasmid showing the basic elements of a cis-plasmid active in the microbiome. No description here is exclusive, the order and content may change as needed, but the same basic elements will remain. The generic plasmid contains one or more sequences conferring an ORI-T phenotype. The ORI-T sequence is activated by the genes encoded by one or more conjugation genes and control elements necessary to activate the ORI-T sequence to initiate conjugation. The generic plasmid contains one or more ORI-V sequences necessary for vegetative replication in one or more host species. The generic plasmid contains one or more cargo genes and the control sequences necessary to express the cargo genes in the recipient hosts. The selection genes contain sequences necessary to maintain the plasmid under selective pressure (a non-exclusive example would include antibiotic resistance genes) in the original or derivatve conjugative hosts or recipients. The generic plasmid may contain genes encoding secondary properties: a non-exclusive example would include genes that modify, augment, repress or degrade any of the sequences noted above. The elements of the generic plasmid are held together by DNA sequences that are used to assemble the elements into one plasmid. These sequences include a mixture of naturally-occurring and synthetically derived sequences commonly known in the art.

FIG. 13: Example of off-target site predictions in the E. coli genome. The sgRNA.off.target.finder.pl inputs a fasta file of sgRNA sequences, searches the sgRNA against a provided reference genome, and outputs (from left to right): the sgRNA on-target site, the predicted off-target site (off_target), the position of the off-target site in the reference genome (OT_pos), the number of nucleotide mismatches relative to the on-target site (num_mm), the number of mis-matches to positions 2 and 3 of the NGG PAM (pam_mm), the mismatch score (mm_score) calculated as described in the Methods, a map of nucleotide mis-matches where asterisks (*) indicate mismatches to the on-target site and dots (.) are nucleotide identities, and a mismatch map for positions 2 and 3 of the PAM se-quence (pam_map) where asterisks (*) are mismatches and dots (.) are identities.

### DESCRIPTION OF THE INVENTION

### Definitions

The practice of the present invention will employ, unless otherwise indicated, conventional techniques of tissue culture, immunology, molecular biology, microbiology, cell biology and recombinant DNA, which are within the skill of the art. See, e.g., Sambrook and Russell eds. (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology.

All numerical designations, e.g., pH, temperature, time, concentration and molecular weight, including ranges, are approximations which are varied (+) or (−) by increments of 1.0 or 0.1, as appropriate, or alternatively by a variation of +/−15%, or alternatively 10%, or alternatively 5% or alternatively 2%. It is to be understood, although not always explicitly stated, that all numerical designations are preceded by the term “about”. It also is to be understood, although not always explicitly stated, that the reagents described herein are merely exemplary and that equivalents of such are known in the art.

As used in the specification and claims, the singular form “a”, “an” and “the” include plural references unless the context clearly dictates otherwise. For example, the term “a polypeptide” includes a plurality of polypeptides, including mixtures thereof.

As used herein, the term “comprising” is intended to mean that the compositions and methods include the recited elements, but do not exclude others. “Consisting essentially of” when used to define compositions and methods, shall mean excluding other elements of any essential significance to the combination for the intended use. Thus, a composition consisting essentially of the elements as defined herein would not exclude trace contaminants from the isolation and purification method and pharmaceutically acceptable carriers, such as phosphate buffered saline, preservatives and the like. “Consisting of” shall mean excluding more than trace elements of other ingredients and substantial method steps for administering the compositions of this invention. Embodiments defined by each of these transition terms are within the scope of this invention.

As used herein “contacting” means any method to deliver the conjugative plasmid to a microbial cell or to a biofilm using standard microbiological or molecular biological techniques including, but not limited to plasmid transformation, conjugation, electroporation, transfection, transduction. The plasmid can be delivered as an isolated DNA or isolated plasmid, or it can be delivered within a system by being carried in another bacterium, bacteriophage, a liposome or any other cell delivery system. The plasmid may also be delivered naked.

A “biofilm” intends to mean a thin layer or an organized community of microorganisms that at times can adhere to the surface of a structure that may be organic or inorganic, together with the polymers, such as polysaccharides, proteins and DNA, that they secrete and/or release. Biofilms are very resistant to microbiotics and antimicrobial agents. They live on gingival tissues, teeth and restorations, causing caries and periodontal disease, also known as periodontal plaque disease. Biofilms are the natural state of the majority of bacteria in contact with any epithelial cell surface. They also cause chronic middle ear infections. Biofilms can also form on the surface of dental implants, stents, catheter lines and contact lenses. They grow on pacemakers, heart valve replacements, artificial joints and other surgical implants. The Centers for Disease Control estimate that over 65% of nosocomial (hospital-acquired) infections are caused by biofilms. Fungal biofilms also frequently contaminate medical devices. They cause chronic vaginal infections and lead to life-threatening systemic infections in people with hobbled immune systems. They occur in life-threatening diseases of the colon such as Clostridium dificile infection. Biofilms also are involved in numerous diseases. For instance, cystic fibrosis patients have Pseudomonas infections that often result in antibiotic resistant biofilms.

A “microbiome” is used in this document as a community of microorganisms (such as bacteria, fungi, archea, viruses and small eukaryotes) that inhabit an organic (including biological) or inorganic surface. In the context of this invention, a microbiome includes any of the above that can accept by conjugation and express the cis-conjugative plasmid of the present invention. Biological surfaces include the human or non-human bodies. Non-biological surfaces may include solid surfaces such as table tops, curtains, filters, industrial tools, industrial bioreactors, environmental surfaces and so forth. The GI tract microbiota has been implicated in disease states such as inflammatory bowel disease, colon cancer, gastric cancer, and irritable bowel syndrome. In addition, a relationship exists between diet, microbiota, and health status, particularly in older subjects.

A “subject” of treatment is a cell or an animal such as a mammal or a human. Non-human animals subject to treatment and are those subject to infections or animal models, for example, simians, murines, such as, rats, mice, chinchilla, canine, such as dogs, leporids, such as rabbits, livestock, sport animals and pets. Non-animal subjects of treatment would include as non-exclusive examples bioreactors, treatment plants, landfills etc.

The term “isolated” or “recombinant” as used herein with respect to nucleic acids, such as DNA or RNA, or plasmids refers to molecules separated from other DNAs or RNAs, respectively that are present in the natural source of the macromolecule as well as polypeptides. The term “isolated or recombinant plasmids” is meant to include plasmids which are not naturally occurring as fragments and would not be found in the natural state. The term “isolated” is also used herein to refer to polynucleotides, polypeptides and proteins that are isolated from other cellular proteins and is meant to encompass both purified and recombinant polypeptides. In other embodiments, the term “isolated or recombinant” means separated from constituents, cellular and otherwise, in which the cell, tissue, polynucleotide, peptide, polypeptide, protein, antibody or fragment(s) thereof, which are normally associated in nature. For example, an isolated cell is a cell that is separated from tissue or cells of dissimilar phenotype or genotype. An isolated polynucleotide is separated from the 3′ and 5′ contiguous nucleotides with which it is normally associated in its native or natural environment, e.g., on the chromosome. As is apparent to those of skill in the art, a non-naturally occurring polynucleotide, peptide, polypeptide, protein, antibody or fragment(s) thereof, does not require “isolation” to distinguish it from its naturally occurring counterpart.

As used herein, the terms “treating,” “treatment” and the like are used herein to mean obtaining a desired pharmacologic and/or physiologic effect. The effect may be prophylactic in terms of completely or partially preventing a disorder or sign or symptom thereof and/or may be therapeutic in terms of a partial or complete cure for a disorder and/or adverse effect attributable to the disorder.

To “prevent” intends to prevent a disorder or effect in vitro or in vivo in a system or subject that is predisposed to the disorder or effect. An example of such is preventing the formation of a biofilm in a system that is infected with a microorganism known to produce one.

“Pharmaceutically acceptable carriers” refers to any diluents, excipients or carriers that may be used in the compositions of the invention. Pharmaceutically acceptable carriers include ion exchangers, alumina, aluminum stearate, lecithin, serum proteins, such as human serum albumin, buffer substances, such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Suitable pharmaceutical carriers are described in Remington's Pharmaceutical Sciences, Mack Publishing Company, a standard reference text in this field. They are preferably selected with respect to the intended form of administration, that is, oral tablets, capsules, elixirs, syrups and the like and consistent with conventional pharmaceutical practices.

“Administration” can be effected in one dose, continuously or intermittently throughout the course of treatment. Methods of determining the most effective means and dosage of administration are known to those of skill in the art and will vary with the composition used for therapy, the purpose of the therapy, the target cell being treated and the subject being treated. Single or multiple administrations can be carried out with the dose level and pattern being selected by the treating physician. Suitable dosage formulations and methods of administering the agents are known in the art. Route of administration can also be determined and method of determining the most effective route of administration are known to those of skill in the art and will vary with the composition used for treatment, the purpose of the treatment, the health condition or disease stage of the subject being treated and target cell or tissue. Non-limiting examples of route of administration include oral administration, nasal administration, injection and topical application.

“Plasmid” refers to an extra-chromosomal DNA molecule separate from the chromosomal DNA. Plasmids replicate extra-chromosomally inside a cell and can transfer their DNA from one cell to another by a variety of mechanisms. DNA sequences controlling extra chromosomal replication (ori) and transfer (tra) are distinct from one another; i.e., a replication sequence generally does not control plasmid transfer, or vice-versa.

A “conjugative plasmid” is a plasmid that is transferred from one organism, such as a bacterial cell, to another organism during a process termed conjugation. The term refers to a self-transmissible plasmid that carries genes promoting the plasmid's own transfer by conjugation. Cis-conjugative plasmids carry their own origin of replication, oriV, and an origin of transfer, oriT, and genes promoting the plasmid's own transfer by the conjugation process. When conjugation is initiated, a relaxase enzyme creates a “nick” in one plasmid DNA strand at the oriT. The enzyme may work alone or in a complex of over a dozen proteins. The transferred, or T-strand, is unwound from the plasmid and transferred into the recipient bacterium in a 5′-terminus to 3′-terminus direction through a conjugative pilus. The remaining strand is replicated, either independent of conjugative action (vegetative replication, beginning at the oriV) or in concert with conjugative replication. Conjugation functions can be plasmid encoded, but some conjugation genes can be found in the bacterial chromosome or another plasmid and can exhibit their activity in trans to a separate plasmid that encodes the oriT sequence. Numerous conjugative plasmids are known, which can transfer associated genes within one species (narrow host range) or between many species (broad host range). Conjugation can occur between species classified as different at any taxonomic level—including in the extreme between domains, e.g. bacteria to eukaryotes.

A cis-conjugative plasmid is a plasmid that encodes both the conjugative machinery and a gene or combination of genes for targeted bacterial modulation, including killing of bacteria (such as CRISPR nuclease), metabolic manipulation of bacteria and augmentation of beneficial bacteria, as well as for the detection of bacteria and so forth.

The term “effective amount” refers to a quantity sufficient to achieve a beneficial or desired result or effect. In the context of therapeutic or prophylactic applications, the effective amount will depend on the type and severity of the condition at issue and the characteristics of the individual subject, such as general health, age, sex, body weight, and tolerance to pharmaceutical compositions. In the context of an immunogenic composition, in some embodiments the effective amount is the amount sufficient to result in a protective response against a pathogen. In other embodiments, the effective amount of an immunogenic composition is the amount sufficient to result in antibody generation against the antigen. In some embodiments, the effective amount is the amount required to confer passive immunity on a subject in need thereof. With respect to immunogenic compositions, in some embodiments the effective amount will depend on the intended use, the degree of immunogenicity of a particular antigenic compound, and the health/responsiveness of the subject's immune system, in addition to the factors described above. The skilled artisan will be able to determine appropriate amounts depending on these and other factors.

In the case of an in vitro application, in some embodiments the effective amount will depend on the size and nature of the application in question. It will also depend on the nature and sensitivity of the in vitro target and the methods in use. The skilled artisan will be able to determine the effective amount based on these and other considerations. The effective amount may comprise one or more administrations of a composition depending on the embodiment.

The agents and compositions can be used in the manufacture of medicaments and for the treatment of humans and other animals by administration in accordance with conventional procedures, such as an active ingredient in pharmaceutical compositions.

An agent of the present invention can be administered for therapy by any suitable route of administration. It will also be appreciated that the preferred route will vary with the condition and age of the recipient and the disease being treated.

The terms “equivalent” or “biological equivalent” are used interchangeably when referring to a particular molecule, biological, or cellular material and intend those having minimal homology while still maintaining desired structure or functionality.

It is to be inferred without explicit recitation and unless otherwise intended, that when the present invention relates to a plasmid, polypeptide, protein, or polynucleotide, an equivalent or a biologically equivalent of such is intended within the scope of this invention. As used herein, the term “biological equivalent thereof” is intended to be synonymous with “equivalent thereof” when referring to a reference protein, antibody, polypeptide or nucleic acid or plasmid, intends those having minimal homology while still maintaining desired structure or functionality. Unless specifically recited herein, it is contemplated that any polynucleotide, polypeptide or protein mentioned herein also includes equivalents thereof. For example, an equivalent intends at least about 70% homology or identity, or alternatively about 80% homology or identity and alternatively, at least about 85%, or alternatively at least about 90%, or alternatively at least about 95% or alternatively 98% percent homology or identity and exhibits substantially equivalent biological activity to the reference protein, polypeptide or nucleic acid. In another aspect, the term intends a polynucleotide that hybridizes under conditions of high stringency to the reference polynucleotide or its complement.

A polynucleotide or polynucleotide sequence (or a polypeptide or polypeptide sequence) having a certain percentage (for example, 80%, 85%, 90% or 95%) of “sequence identity” to another sequence means that, when aligned, that percentage of bases (or amino acids) are the same in comparing the two sequences. The alignment and the percent homology or sequence identity can be determined using software programs known in the art, for example those described in Current Protocols in Molecular Biology (Ausubel et al., eds. 1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 30% identity or alternatively less than 25% identity, less than 20 identity, or alternatively less than 10% identity with one of the sequences of the present invention.

“Homology” or “identity” or “similarity” can also refer to two nucleic acid molecules that hybridize under stringent conditions to the reference polynucleotide or its complement.

Overview

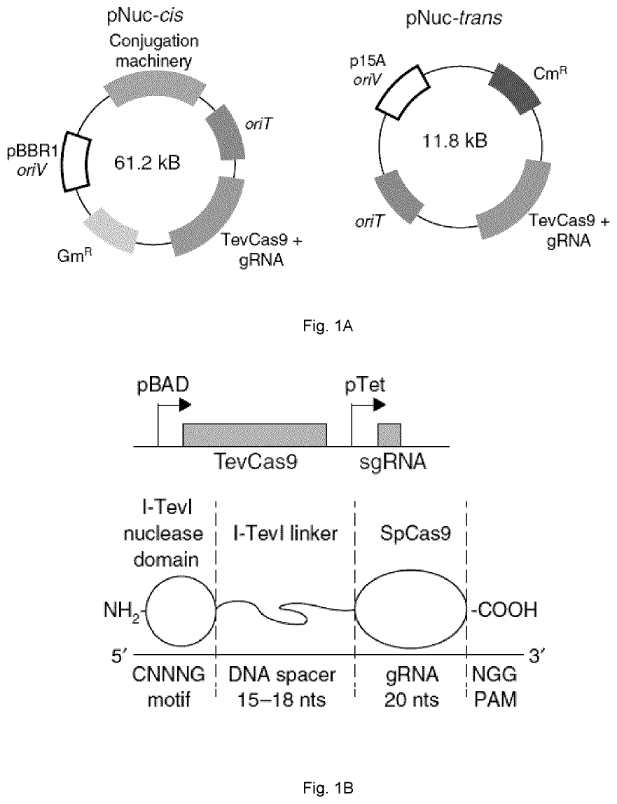

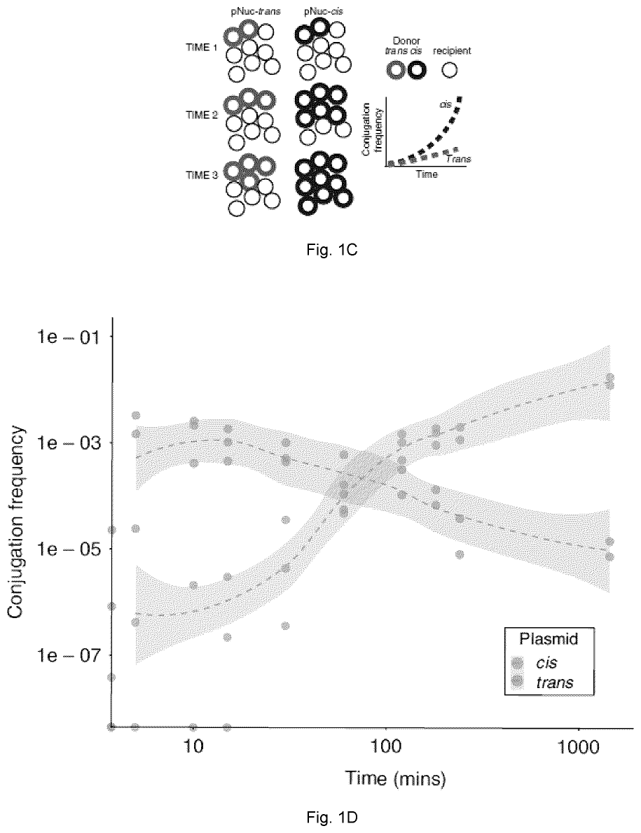

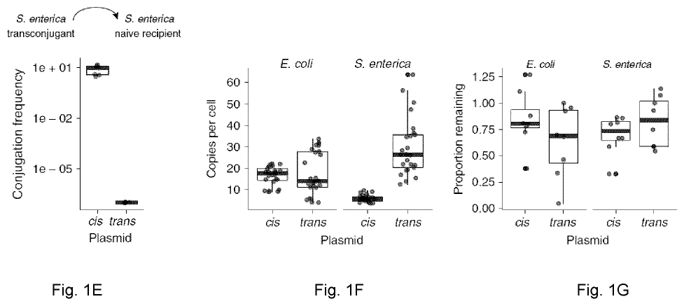

Provided herein is a new cis-conjugative plasmid system and method of using said cis-conjugative plasmid system in altering or modulating or modifying a bacterial microbiome, including biofilms. In one embodiment, the cis-conjugative plasmid of the present invention encodes both the conjugative machinery and a gene or genes of interest that is/are capable of being expressed in a target bacteria species of interest within a microbiome or biofilm, and that serves to alter or modulate only the target bacteria species in the microbiome or biofilm, as opposed to previously tested trans setups where the conjugation machinery and gene of interest were separated (FIG. 1). Any bacterium in the bacterial microbiome/biofilm that receives a cis-conjugative plasmid of the present invention becomes a donor for subsequent rounds of re-conjugation, leading to exponentially increasing numbers of conjugative donor bacteria in a population (or biofilm) of bacteria such as a microbiome carrying the gene of interest, however, only the abundance or cellular physiology of the target bacteria in the microbiome or biofilm will be directly modulated by the gene of interest. The cis-conjugative plasmid of the present invention is highly efficient in conjugative transfer among the different bacteria in the microbiome, including in a biofilm.

Applications

The gene or genes of interest may be a gene or genes that alters, modifies, modulates or manipulates the bacteria, or a subpopulation of bacteria in the bacterial microbiome or biofilm. The cis-conjugative plasmid of the present invention may include a gene or combination of genes to target specific bacteria within a population of different bacterial species. While any bacterium in the bacterial microbiome/biofilm that receives the cis-conjugative plasmid of the present invention becomes a donor for subsequent rounds of re-conjugation, leading to exponentially increasing numbers of conjugative donor bacteria in a population of bacteria such as a microbiome or a biofilm carrying the gene of interest, only the target specific bacteria within the population is modulated. The applicant surprisingly discovered a high degree of efficiency in the conjugative transfer of the cis-conjugative plasmid of the present invention intra-species and inter-species of bacteria. As such, the systems and methods of the present invention can be used as effective tools in the manipulation of microbiomes. The present invention also relates to cis-conjugated plasmids engineered so that the gene product is only active in a target bacteria.

The gene or combination of genes of interest may include genes that lead to the killing of the target bacteria, or to the growth of beneficial bacteria, or to the production of molecules of interest and so forth. The gene or combination of genes may include inducible genes that are turned on and off when certain conditions are met. For example, pH and temperature may change along the Gastrointestinal (GI) tract. pH or Temperature-sensitive genes having permissive and non-permissive pHs/temperatures could be used to deliver the plasmids of the present invention orally to a target segment of the GI tract, without having activation of the plasmid before reaching the target segment of the GI tract.

The following is a non-exhaustive list of modulations that can be manipulated with the systems of the present invention.

1. Elimination of harmful bacteria. The cis-conjugative plasmid of the present invention may include a gene or combination of genes that target specific bacteria, within a microbiome, and eliminate said specific bacteria. A non-limiting example of said gene or genes, include the gene that encodes for the TevCas9 nuclease specifically repurposed for killing specific bacteria species within a population of different bacteria species.

2. Augmentation of beneficial microbes in a microbiome. A non-limiting example would be introduction of novel biosynthetic or biodegradative pathways by the cis-conjugative plasmid to enhance growth of the beneficial microbe. A second non-limiting example would be delivery of metabolic capacity to the cis-conjugative plasmid to difficult to cultivate bacteria.

3. Metabolic manipulation of a microbiome by introduction of regulatory sequences by the cis-conjugative plasmid, including but not limited to small RNA molecules and transcription factors, to modulate expression of a gene or genes that are encoded by the target bacteria species that control biosynthesis or degradation of a metabolic product.

Administration

The cis-conjugative plasmid of the present invention is introduced by standard microbiological techniques (plasmid transformation, conjugation, electroporation, transfection, transduction, etc) into a bacterial species, such as a bacterial species that is generally recognized as safe (GRAS). This would include any species that is currently used as a probiotic or used as a food supplement or that can be introduced into an industrial setting or any other environment. The GRAS bacteria is the donor for conjugation of the cis-conjugative plasmid to the microbiome. Administration specifically refers to the bacteria, such as GRAS bacteria, containing the cis-conjugative plasmid that may be administered by a method comprising topically, transdermally, sublingually, rectally, vaginally, ocularly, subcutaneously, intramuscularly, intraperitoneally, urethrally, intranasally, by inhalation or orally. In the instance of non-animal administration, the cis-conjugative plasmid could be introduced as an inoculum into an industrial or environmental system.

In some aspects, the subject is a pediatric patient and the cis-conjugative plasmid is administered in a formulation for the pediatric patient.

In one embodiment, the cis-conjugative plasmid of the present invention is administered locally to the microbial infection.

The cis-conjugative plasmid of the present invention can be concurrently or sequentially administered with other antimicrobial agents and/or surface antigens. In one particular aspect, administration is locally to the site of the infection. Other non-limiting examples of administration include by one or more method comprising transdermally, sublingually, rectally, vaginally, ocularly, intranasally, by inhalation or orally.

Microbial infections and disease that can be treated by the methods of this invention include infection by, for example, Streptococcus agalactiae, Neisseria meningitidis, Treponemes, denticola, pallidum, Burkholderia cepacia or Burkholderia pseudomallei. In one aspect, the microbial infection is one or more of Haemophilus influenzae (nontypeable), Moraxella catarrhalis, Streptococcus pneumoniae, Streptococcus pyogenes, Pseudomonas aeruginosa, Mycobacterium tuberculosis. These microbial infections may be present in the upper, mid or lower airway (otitis, sinusitis or bronchitis) but also exacerbations of chronic obstructive pulmonary disease (COPD), chronic cough, complications of and/or primary cause of cystic fibrosis (CF) and community acquired pneumonia (CAP).

Infections might also occur in the oral cavity (caries, periodontitis) and caused by Streptococcus mutans, Porphyromonas gingivalis, Aggregatibacter actinomycetemcomitans. Infections might also be localized to the skin (abscesses, ‘staph’ infections, impetigo, secondary infection of burns, Lyme disease) and caused by Staphylococcus aureus, Staphylococcus epidermidis, Pseudomonas aeruginosa and Borrelia burdorferi. Infections of the urinary tract (UTI) can also be treated and are typically caused by Escherichia coli. Infections of the gastrointestinal tract (GI) (diarrhea, cholera, gall stones, gastric ulcers) are typically caused by Salmonella enterica serovar, Vibrio cholerae and Helicobacter pylori. Infections of the genital tract include and are typically caused by Neisseria gonorrhoeae. Infections can be of the bladder or of an indwelling device caused by Enterococcus faecalis. Infections associated with implanted prosthetic devices, such as artificial hip or knee replacements or dental implants or medical devices such as pumps or monitoring systems, typically caused by a variety of bacteria, can be treated by the methods of this invention. These devices can be coated or conjugated to the cis-conjugative plasmid of the present invention.

Infections caused by Streptococcus agalactiae are the major cause of bacterial septicemia in newborns. Such infections can also be treated by the methods of this invention. Likewise, infections caused by Neisseria meningitidis which can cause meningitis can also be treated.

Thus, routes of administration applicable to the methods of the invention include intranasal, intramuscular, intratracheal, subcutaneous, intradermal, topical application, intravenous, rectal, nasal, oral and other enteral and parenteral routes of administration. Routes of administration may be combined, if desired, or adjusted depending upon the agent and/or the desired effect. The cis-conjugative plasmid of the present invention can be administered in a single dose or in multiple doses. Embodiments of these methods and routes suitable for delivery, include systemic or localized routes. In general, routes of administration suitable for the methods of the invention include, but are not limited to, enteral, parenteral or inhalational routes.

Parenteral routes of administration other than inhalation administration include, but are not limited to, topical, transdermal, subcutaneous, intramuscular, intraorbital, intracapsular, intraspinal, intrasternal and intravenous routes, i.e., any route of administration other than through the alimentary canal. Parenteral administration can be conducted to effect systemic or local delivery of the inhibiting agent. Where systemic delivery is desired, administration typically involves invasive or systemically absorbed topical or mucosal administration of pharmaceutical preparations.

The cis-conjugative plasmid of the present invention can also be delivered to the subject by enteral administration. Enteral routes of administration include, but are not limited to, oral and rectal (e.g., using a suppository) delivery.

Methods of administration of the cis-conjugative plasmid of the present invention through the skin or mucosa include, but are not limited to, topical application of a suitable pharmaceutical preparation, transcutaneous transmission, transdermal transmission, injection and epidermal administration. For transdermal transmission, absorption promoters or iontophoresis are suitable methods. Iontophoretic transmission may be accomplished using commercially available “patches” that deliver their product continuously via electric pulses through unbroken skin for periods of several days or more.

In various embodiments of the methods of the invention, the cis-conjugative plasmid of the present invention will be administered orally on a continuous, daily basis, at least once per day (QD) and in various embodiments two (BID), three (TID) or even four times a day. For example, a minimum of 10⁹ CFU/ml of GRAS species having the cis-conjugative plasmid of the present invention may be administered as a dosage.

Dosing of can be accomplished in accordance with the methods of the invention using capsules, tablets, oral suspension, gel or cream for topical application. In the instance of non-human, non-animal administration, the dosing can be accomplished by suspension, tablets, gel or cream.

The skilled artisan will appreciate that certain factors may influence the dosage and timing required to effectively treat a subject, including but not limited to, the severity of the disease or disorder, previous treatments, the general health and/or age of the subject, and other diseases present. Moreover, treatment of a subject with a therapeutically effective amount of the therapeutic compositions described herein can include a single treatment or a series of treatments.

The compositions and related methods of the present invention may be used in combination with the administration of other therapies. These include, but are not limited to, the administration of DNase enzymes, antibiotics, antimicrobials, or other antibodies.

Kits

Kits containing the agents and instructions necessary to perform in vitro and in vivo methods as described herein also are claimed. Accordingly, the invention provides kits for performing these methods which may include a cis-conjugative plasmid of the present invention as well as instructions for carrying out the methods of this invention such as collecting tissue and/or performing the screen and/or analyzing the results and/or administration of an effective amount of biological agent as defined herein. These can be used alone or in combination with other suitable antimicrobial agents.

In another embodiment, the cis-conjugative plasmid of the present invention can be used in the detection of a target bacteria within a microbiome or biofilm or in the diagnosis of an infectious disease or condition. The guide RNA included in the cis-conjugative plasmid of the present invention may serve to detect a target bacteria in a microbiome or biofilm.

In one embodiment, the present application enables the tracking or detection of Clostridium difficile by transposon-based tagging. The transposon would be delivered by the cis-conjugative plasmid of the present invention and be engineered to only target C. difficile.

The cis-conjugative plasmid of the present invention can be used for tracking uncultivatable bacteria (and also pathogens such as C. difficile) that can be present in very low relative abundance in microbiomes yet have significant contributions to the microbial community. In one embodiment, CRISPR-guided transposons encoded on a cis-conjugative plasmid that would insert only in genes specific to the bacterium of interest. This transposon could encode, for example, a label, such as a fluorescent reporter (such as green fluorescent protein GFP) such that tagged bacteria could be isolated by fluorescent activated cell sorting for downstream attempts at cultivation, or for molecular-based studies as such RNAseq or metagenomics.

The cis-conjugative plasmid of the present invention has numerous potential applications beyond targeted specific bacteria for elimination using CRISPR. The cloning capacity of the cis-conjugative plasmid is very large (at least up to 800 kb sized inserts) meaning that cargo can range from single genes, entire biosynthetic pathways, or whole genomes. As such, the present invention enables the cis-conjugative plasmid for delivery of molecular tools for engineering microbial genomes in situ, for modulating the metabolic output of the human gut microbiome (or any microbiome) by adding additional metabolic capacity, for modulating the expression of existing pathways, or for molecular diagnostic purposes by tracking specific bacteria within complex populations. Any microbiome that is permissible to conjugation is amenable to manipulation through the delivery of genetically-encoded molecular agents. Potential applications could include (but not limited to) modifying the metabolic output of a microbiome, such as the gut microbiome, for increased tolerance to chemotherapeutic agents or tracking the dynamics of pathogens, such as Clostridium difficile, by transposon-based tagging.

### EXAMPLES

These Examples are described solely for purposes of illustration and are not intended to limit the scope of the invention. Changes in form and substitution of equivalents are contemplated as circumstances may suggest or render expedient.

### Example 1—High Efficiency Inter-Species Conjugative Transfer of a CRISPR Nuclease for Targeted Bacterial Elimination

Materials and Methods

Bacterial Strains and Plasmid Construction

E. coli EPI300 (Epicentre) was used for cloning and as a conjugative donor (F′ λ-mcrA Δ(mrr-hsdRMS-mcrBC) ϕ80dlacZ ΔM15 Δ(lac)X74 recA1 endA1 araD139 Δ(ara, leu)7697 galU galK rpsL (Str^{R}) nupG trfA dhfr). Salmonella typhimurium sub. species enterica LT2 (acquired from Dr. David Haniford at Western University) was used as a conjugative recipient strain.

Plasmid Construction.

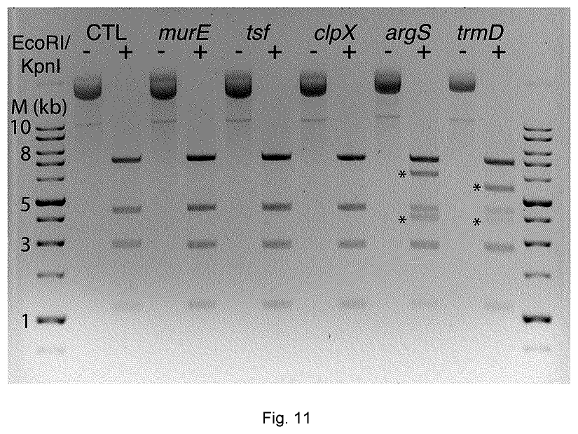

Plasmids were constructed using a modified yeast assembly. A list of primers is provided. Table 1. The pNuctrans plasmid was constructed by polymerase chain reaction (PCR) amplification of fragments with 60-120 bp homology overlaps from pre-existing plasmids. The oriT fragment was amplified from pPtGE3052 using primers DE-3302 and DE-3303. The p15A origin, chloramphenicol acetyl-transferase gene, and sgRNA cassette was amplified using primers DE-3308 and DE-3309 from a modified pX458 plasmid containing the TevSpCas9 coding region. The TevSpCas9 gene was amplified from the modified pX458 plasmid using primers DE-3306 and DE-3307. The araC gene and pBAD promoter were amplified from pBAD-24 using primers DE-3304 and DE-3305. The CEN6-ARSH4-HIS3 yeast element was amplified from pPtGE30 using primers DE-3316 and DE-3317. S. cerevisiae VL6-48 was grown from a single colony to an OD₆₀₀ of 2.5-3, centrifuged at 2500×g for 10 min and washed in 50 mL sterile ddH20 and centrifuged. Cells were resuspended in 50 mL of 1M sorbitol, centrifuged, and spheroplasting initiated by resuspending the pellet in 20 mL SPE solution (1M sorbitol, 10 mM sodium phosphate buffer pH 7, 10 mM Na2EDTA pH 7.5) and by adding 30 μL 12M 2-mercaptoethanol and 40 μL zymolyase 20T solution (200 mg zymolyase 20T (USB), 9 mL H2O, 1 mL 1M Tris pH 7.5, 10 mL 50% glycerol) and incubated at 30° C. with shaking at 75 RPM. The yeast was considered spheroplasted once the ratio of the OD₆₀₀ in sorbitol to the OD₆₀₀ of yeast in ddH20 reached 1.8-2. Spheroplasts were centrifuged at 1000×g for 5 min before being gently resuspended in 50 mL 1M sorbitol, and centrifuged again. Spheroplasts were then resuspended in 2 mL STC solution (1M sorbitol, 10 mM Tris-HCl pH 7, 10 mM CaCl₂)) and incubated at room temperature for 10 min. Pooled DNA fragments at equimolar ratio for each plasmid assembly were gently mixed with 200 μL of spheroplasted yeast and incubated at room temperature for 10 min. A volume of 1 mL of PEG-8000/CaCl₂) solution (20% (w/v) PEG 8000, 10 mM CaCl₂), 10 mM Tris-HCl, pH 7.5) was added and incubated at room temperature for 20 min before being centrifuged at 1500×g for 7 min. Yeast was resuspended in 1 mL of SOS solution (1M sorbitol, 6.5 mM CaCl₂), 0.25% (w/v) yeast extract, 0.5% (w/v) peptone) and incubated at 30° C. for 30 min. The spheroplast solution was added to 8 mL of histidine-deficient regenerative agar (Teknova), poured into a petri dish, and incubated overnight at 30° C. A volume of 8 mL histidine-deficient liquid regenerative media was then added on top of the solidified regenerative agar and grown at 30° C. for 2-5 days. Total DNA was isolated from 1.5 to 3 mL S. cerevisiae using 250 μL buffer P1 (50 mM Tris-HCl pH 8.0, 10 mM EDTA, 100 μg/mL RNase A), 12.5 μL zymolyase 20 T solution and 0.25 μL 12M 2-mercaptoethanol and incubated at 37° C. for 1 h. In total, 250 μL buffer P2 (200 mM NaOH, 1% sodium dodecyl sulfate) was added, incubated at room temperature for 10 min, followed by addition of 250 μL buffer P3 (3.0M CH3CO2K pH 5.5). DNA was precipitated with 700 μL ice-cold isopropanol, washed with 70% ethanol, briefly dried and resuspended in 50 μL sddH2O. The plasmid pool was subsequently electroporated into E. coli EPI300. Individual colonies were screened by diagnostic digest (FIG. 11) and sequencing (Table 5), and one clone for each sgRNA selected for further use. TevSpCas9 sgRNAs targeting S. enterica genes were predicted as previously described. A TevSpCas9 site consists of (in the 5′ to 3′ direction) an I-Tevl cleavage motif (5′-CNNNG-3′), a DNA spacer region of 14-19 bp separating the I-Tevl cleavage site and the SpCas9 sgRNA binding site, and a SpCas9 PAM site (5′-NGG-3′). Putative sites in the S. enterica LT2 genome were ranked according to the predicted activity of the identified I-Tevl cleavage site (relative to the I-Tevl cognate 5′-CAACG-3′ cleavage site) and the fit of the DNA spacer region to nucleotide tolerances of ITevl. Oligonucleotides corresponding to the guide RNA were cloned into a Bsal cassette site present in pNuc-trans. To construct the pNuc-cis plasmid, the oriT, araC, TevCas9, sgRNA, and CEN6-ARSH4-HIS3 elements were amplified from pNuc-trans using primers DE-3024 and DE-3025 that possessed 60 bp homology to both sides of the Avrll restriction site in pTA-Mob. The pTA-Mob plasmid was linearized by Avrll (New England Biolabs), combined with the PCR amplified fragment from pNuc-trans and transformed into S. cerevisiae VL6-48 spheroplasts. Correct pNuc-cis clones were identified as above for pNuc-trans. Both pNuc-trans and pNuc-cis were completely sequenced to confirm assembly. A detailed plasmid map and sequence of each plasmid is provided as Table 3 and 4.

The entire nucleic acid sequence of pNuc-cis is provided in Table 3 as SEQ ID NO:27.

Quantitative PCR.

E. coli EPI300 donors and S. enterica transconjugants harboring pNuc-trans and pTA-Mob (trans helper plasmid) or pNuc-cis were grown overnight under selection. sgRNAs were absent from the cis and trans plasmids.

Overnight cultures were diluted 1:50 in selective media and grown to an A₆₀₀ of ˜0.5. Each culture was diluted, plated on selective LSLB plates (10 g/L tryptone, 5 g/L yeast extract, and 5 g/L sodium chloride, 1% agar), and grown overnight. Colonies were counted manually to determine the CFUs/mL of each culture. At the same time, 500 μL of each culture was pelleted and resuspended in 500 μL 1× phosphate-buffered saline (PBS) and incubated at 95° C. for 10 min before immediate transfer to −20° C. Quantitative real-time PCR was performed on boillysed samples using SYBR Select Master Mix (Applied Biosystems) using primers DE-4635 and DE-4636 that amplified a DNA fragment present on both pNuc-trans and pNuc-cis. Purified pNuc-trans was used as a copy number standard.

Filter Mating Conjugation.

Saturated cultures of donor E. coli EPI300 and recipient S. enterica LT2 were diluted 1:50 into 50 mL nonselective LSLB media. The diluted cultures were grown to an A₆₀₀ of ˜0.5 and concentrated 100-fold by centrifugation at 4000×g for 10 min. A volume of 200 μL of concentrated donors were mixed with 200 μL concentrated recipients on polycarbonate filters adhered to conjugation plates (LSLB supplemented with 1.5% agar). Conjugation proceeded at 37° C. from 5 min to 24 h. Following conjugation, filters were placed in conical tubes containing 30 mL of 1×PBS (8 g/L NaCl. 0.2 g/L KCl, 1.42 g/L Na₂HPO₄, 0.24 g/L KH₂PO₄) and vortexed for 1 min to remove the bacteria from the filter. The supernatant was serially diluted and plated on LSLB plates with selection for donor E. coli EPI300 (gentamicin 40 μg/mL for the cis setup and gentamicin 40 μg/mL, chloramphenicol 25 μg/mL for the trans setup), recipient S. enterica LT2 (kanamycin 50 μg/mL), and transconjugants (kanamycin 50 μg/mL, chloramphenicol 25 μg/mL, 0.2% D-glucose for for pNuc-trans transconjugants or kanamycin 50 μg/mL, gentamicin 40 μg/mL, 0.2% D-glucose for pNuc-cis transconjugants). D-glucoserepresses the expression of TevCas9 in transconjugants. Plates were incubated overnight at 37° C. for 16-20 h. Colonies were counted manually.

S. enterica to S. enterica conjugation.

S. enterica LT2 transconjugants harboring pNuc-cis or pNuc-trans with no sgRNA encoded were obtained from plate conjugation experiments described in detail in the supplementary methods. Transconjugant colonies were grown overnight in LSLB supplemented with kanamycin 50 μg/mL, gentamicin 40 μg/mL and 0.2% D-glucose for pNuc-cis, or kanamycin 50 μg/mL, chloramphenicol 25 μg/mL and 0.2% D-glucose for pNuctrans. S. enterica LT2 was transformed with pUC19 to confer ampicillin resistance for use as a recipient and was grown overnight in LSLB supplemented with kanamycin 50 μg/mL and ampicillin 100 μg/mL. All donor and recipient S. enterica cultures were diluted 1:50 into LSLB and grown to an A₆₀₀ of 0.5 before spreading 200 μL of each on a conjugation plate supplemented with 0.2% w/v D-glucose to repress TevSpCas9 expression. Conjugations proceeded for 2 h at 37° C. before cells were scraped into 500 μL SOC with a cell spreader. Resulting cell suspensions were serially diluted and plated to select for donors (kanamycin 50 μg/mL, gentamicin 25 μg/mL for pNuc-cis or kanamycin 50 μg/mL, chloramphenicol 25 μg/mL for pNuc-trans), recipient (kanamycin 50 μg/mL, ampicillin 100 μg/mL), and transconjugant (kanamycin 50 μg/mL, gentamicin 40 μg/mL, ampicillin 100 μg/mL for pNuc-cis, chloramphenicol 25 μg/mL, ampicillin 100 μg/mL for pNuc-trans). Plates were incubated at 37° C. for 16-20 h and colonies were counted manually.

Liquid and Bead-Supplemented Conjugation Assays.

E. coli EPI300 and recipient S. enterica LT2 were grown overnight to saturation. Tubes containing 5 mL LSLB supplemented with 0.2% D-glucose were inoculated with 180 μL saturated E. coli and 18 μL saturated S. enterica. Bead-supplemented conjugations were prepared similarly with the addition of 1 mL soda lime glass beads (0.5 mm diameter). Conjugations proceeded by incubating at 37° C. with 0 or 60 RPM agitation for 72 h. Cultures were homogenized by vortexing, serially diluted and spot-plated in 10 μL spots on plates containing appropriate antibiotic selection for donors, recipients, and transconjugants. Plates were incubated at 37° C. for 16-20 h. Colonies were counted manually. Alterations to this protocol were made to determine the effect of donor to recipient ratio (50:1, 10:1, 1:1, 1:10, 1:50), NaCl concentration (2.5, 5, and 10 g/L) and shaking speed (0, 60, and 120 RPM) on conjugation frequency. Killing efficiency assays. Saturated cultures of E. coli EPI300 donors habouring pNuc-trans plasmids encoding sgRNAs and recipient S. enterica LT2 were diluted 1:50 into LSLB supplemented with 0.2% D-glucose. The diluted cultures were grown to an A₆₀₀ of ˜0.5. 200 μL of each donor was mixed with 200 μL of recipient on a conjugation plate supplemented with 0.2% D-glucose to repress expression of TevCas9. Conjugations proceeded for 1 h at 37° C. before cells were scraped into 500 μL SOC (20 g/L tryptone, 5 g/L yeast extract, 0.5 g/L NaCl, 2.5 mM KCl, 10 mM MgCl2, and 20 mM D-glucose) with a cell spreader. Resulting cell suspensions were serially diluted and plated on selection for donors and recipients in addition to selection for transconjugants with CRISPR repression (kanamycin 50 μg/mL, chloramphenicol 25 μg/mL, 0.2% D-glucose) and transconjugants with CRISPR activation (kanamycin 50 μg/mL, chloramphenicol 25 μg/mL, 0.2% L-arabinose). Plates were incubated overnight at 37° C. for 16-20 h. Killing efficiency is the ratio of cells on selective to nonselective plates.

Escape Mutant Analyses.

Escape mutant colonies were picked from plates selecting for exconjugant S. enterica cells with TevSpCas9 activated after conjugation. These colonies were grown overnight to saturation and plasmids were extracted using the BioBasic miniprep kit. The isolated plasmids were then electroporated into E. coli EPI300 cells and re-isolated for analysis. The plasmids were analyzed by diagnostic restriction digest with FspI and Msil, and by multiplex PCR for the chloramphenicol resistance marker, and a TevSpCas9 gene fragment. Total DNA was isolated using a standard alkaline lysis protocol followed by isopropanol precipitation of the DNA. Potential target sites were PCR amplified from the total DNA sample using Amplitaq 360 (Thermofisher Scientific) and subsequently sequenced.

sgRNA Off-Target Predictions in E. coli.

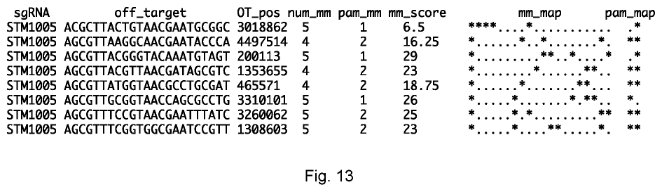

To predict sgRNA off-target sites, we searched the E. coli genome for sites with less than six mismatches to each sgRNA using a Perl script with an XOR bit search. A mismatch score was calculated that indicates the likelihood of a stable sgRNA/DNA heteroduplex using the formula

$$mm\_score = {\sum }_{mismatch} {0.5}^{non\_seed} + 1 . {2}^{seed} ,$$

where non_seed is a mismatch in the nonseed region of the sgRNA (positions 1-12 from the 5′ end of the target site) and seed is a mismatch in the seed regions (positions 13-20 from the 5′ end of the target site). By this method, mismatches in the 5′ end of sgRNA/DNA heteroduplex are more tolerated than mismatches closer to the PAM sequence. For each sgRNA, we also added a correction for if the adjacent three nucleotides matched the consensus SpCas9 PAM sequence 5′-NGG-3′. Off-target sites with perfect match PAMs were given more weight than offtarget sites with 1 or 2 mismatches. Sample fasta formatted files of sgRNAs (sgRNA.test.fa) and an E. coli genome (MG16552.fna) are also provided. Source code and instructions to execute the perl script are provided in Hamilton et al. (2019) Efficient inter-species conjugative transfer of a CRISPR nuclease for targeted bacterial killing. Nature Communications, 10: 4544. A sample output is shown in FIG. 13.
Modeling S. enterica Killing Efficiency.
To model sgRNA parameters that were predictive of S. enterica killing efficiency, we used a generalized linear model in the R statistical language with the formula

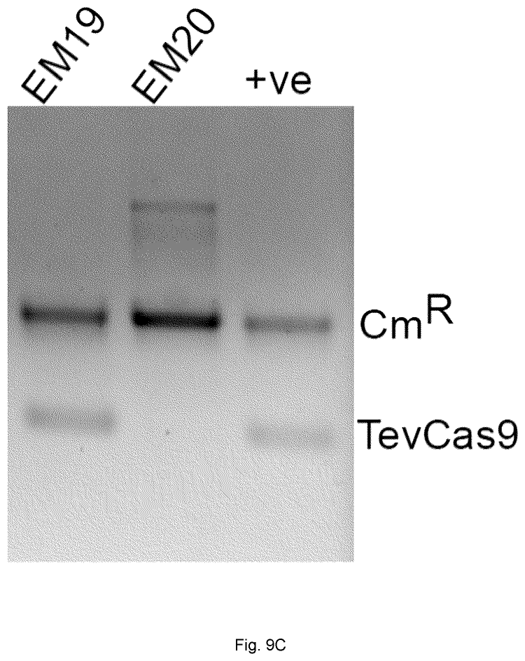

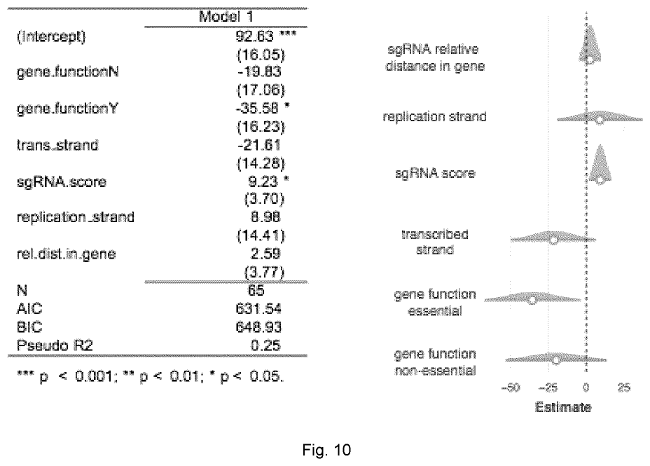

sgRNA_{KE}˜sgRNA_{score}+sgRNA_{target strand}+sgRNA_{repstrand}+sgRNA_{gene func}+sgRNA_{reldist},

where sgRNA_{KE} is the average killing efficiency for a given sgRNA, sgRNA_{score} is the predicted sgRNA activity score using the algorithm of Guo et al. (Nucleic Acids Res. 46, 7052-7069 (2018)), sgRNA_{targetstrand} is the transcription strand targeted by the sgRNA (sense or antisense), sgRNA_{repstrand} is whether the sgRNA targets the leading or lagging strand, sgRNA_{genefunc} is whether the sgRNA targets an essential or non-essential gene in S. enterica, and sgRNA_{reildist} is the position of the sgRNA relative to the AUG codon of the targeted gene. A summary table and graphical output of the model parameters is shown in FIG. 10.

Results

Increased Conjugation Frequency with a Cis-Conjugative Plasmid.

We constructed a conjugative plasmid, pNuc, based on the IncP RK2 plasmid to examine parameters that contributed to conjugation (FIG. 1A). The pNuc plasmid encoded the TevSpCas9 nuclease (I-Tevl nuclease domain fused to Streptococcus pyogenes Cas9) controlled by an arabinose-inducible pBAD promoter, and a single-guide RNA (sgRNA) cassette driven by a constitutive promoter derived from the tetracycline resistance gene (pTet) into which we cloned oligonucleotides corresponding to predicted target sites in the S. enterica genome (FIG. 1B). Two forms of the plasmid were constructed (FIG. 1A). First, a cis configuration (pNuc-cis) where the origin of transfer (oriT) and CRISPR system were cloned into the pTA-Mob backbone that encodes the genes necessary for conjugation. The second setup employed a plasmid trans configuration (pNuc-trans) that included only the CRISPR system, oriT, and chloramphenicol resistance. The oriT sequence on pNuc-trans is recognized by the relaxase expressed in trans from the pTA-Mob helper plasmid to facilitate conjugation. The pNuc-trans setup mimics the plasmids used in previous studies that examined conjugative delivery of CRISPR nucleases in an E. coli donor/recipient system.

We used the pNuc-cis and pNuc-trans plasmids to test the hypothesis that the cis setup would support higher levels of conjugation relative to the trans setup in a time-course filtermating assay using E. coli as the donor and S. enterica as the recipient. As shown in FIG. 1C, conjugation frequency (transconjugants/total recipients) for pNuc-cis continually increased over the time of the experiment reaching a maximum of 1×10⁻² by 24 h. In contrast, conjugation frequency for pNuc-trans peaked at early time points with a maximal frequency of ˜1×10⁻³, declining to ˜1×10⁻⁵ by 24 h. We isolated five S. enterica transconjugants each from experiments with the pNuc-cis or pNuc-trans plasmids and showed that the transconjugants were viable donors for subsequent conjugation of the pNuc-cis plasmid to naive recipients, but not for the pNuc-trans plasmid (FIG. 1D). Furthermore, higher frequency conjugation of pNuc-cis was not due to higher copy number relative to pNuc-trans in the E. coli donor or S. enterica transconjugants (FIG. 1E), or because pNuc-cis was significantly more stable than pNuc-trans (FIG. 1F).

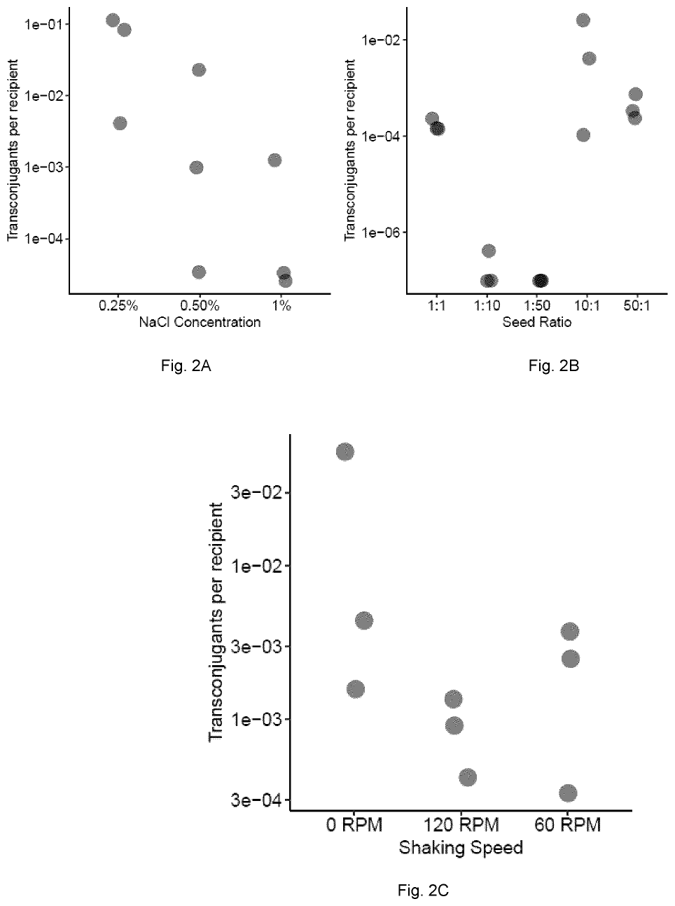

To determine if longer incubation times resulted in higher conjugation frequency with the pNuc-cis system, we used a liquid conjugation assay consisting of low-salt LB (LSLB) media into which varying ratios of donor E. coli and recipient S. enterica cells were added. After 72 h incubation at 37° C. with mild agitation at 60 RPM, we found that high donor to recipient ratios (1:1, 10:1, and 50:1) yielded more transconjugants per recipient than experiments with lower donor to recipient ratios (1:5 or 1:10) (FIG. 2A). We also showed that decreasing the NaCl concentration of the media to 0.25% w/v resulted in an increased conjugation frequency at a 10:1 donor:recipient ratio (FIG. 2B). Using the 10:1 donor:recipient ratio, and 0.25% NaCl LSLB media, we examined the effect of culture agitation on conjugation, finding that both 0 and 60 RPM resulted in similar conjugation frequencies while a higher 120 RPM resulted in lower conjugation frequency (FIG. 2C).

Collectively, these data show that pNuc-cis has an ˜1000-fold higher conjugation frequency than the pNuc-trans system at 24 h post-mixing because bacteria that receive pNuc-cis become donors for subsequent rounds of conjugation. This would lead to exponentially increasing numbers of conjugative donors in the population. Thus, our data differ significantly from previous studies that concluded that conjugation frequency with a trans system was a limiting factor for CRISPR delivery.

Cell-to-Cell Contact Significantly Increases Conjugation.

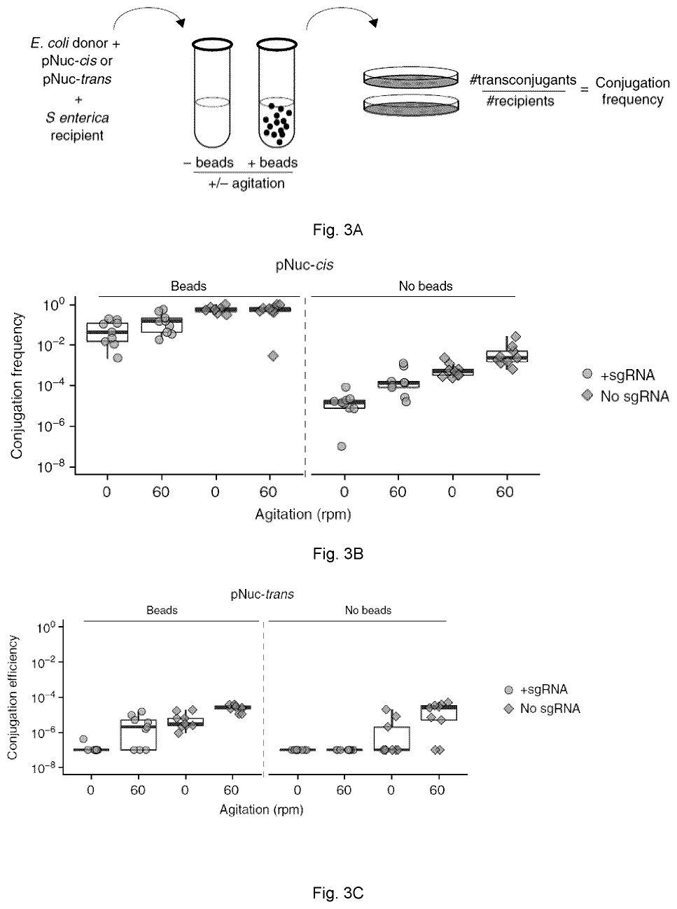

The previous experiments demonstrated that pNuc-cis was more efficient at conjugation in a filter mating assay on solid media. With reference to FIG. 3A, to test whether liquid culture conditions that enhanced cell-to-cell contact through biofilm formation resulted in increased conjugation with pNuc-cis, we included 0.5 mm glass beads in liquid cultures that would provide a solid surface for cell-to-cell contact and observed conjugation frequencies as high as 100% with pNuc-cis (FIG. 3B). This conjugation frequency represents a ˜500- to 1000-fold enhancement compared to the solution or filter-based pNuc-cis assays. Increasing culture agitation to 60 RPM had no discernible effects on conjugation frequency with pNuc-cis. With the pNuc-trans plasmid, conjugation frequency ranged from 1×10⁻⁸ to 1×10⁻⁴ (FIG. 3B), supporting the hypothesis that gains in conjugation frequency with the pNuc-cis system resulted from exponentially increasing number of cells that become donors for subsequent rounds of conjugation after receiving the plasmid.

Interestingly, we observed a reduction in conjugation frequency when a S. enterica specific sgRNA was cloned onto pNuc-cis (the +guide condition) (FIGS. 3B and 3C, filled circles). We postulate that a proportion of S. enterica are killed immediately post-conjugation. We attribute this killing to leaky expression of the TevSpCas9 nuclease from the pBAD promoter under repressive culture conditions (+0.2% glucose).

S. enterica Killing by Conjugative Delivery of Cas9 and sgRNAsS.

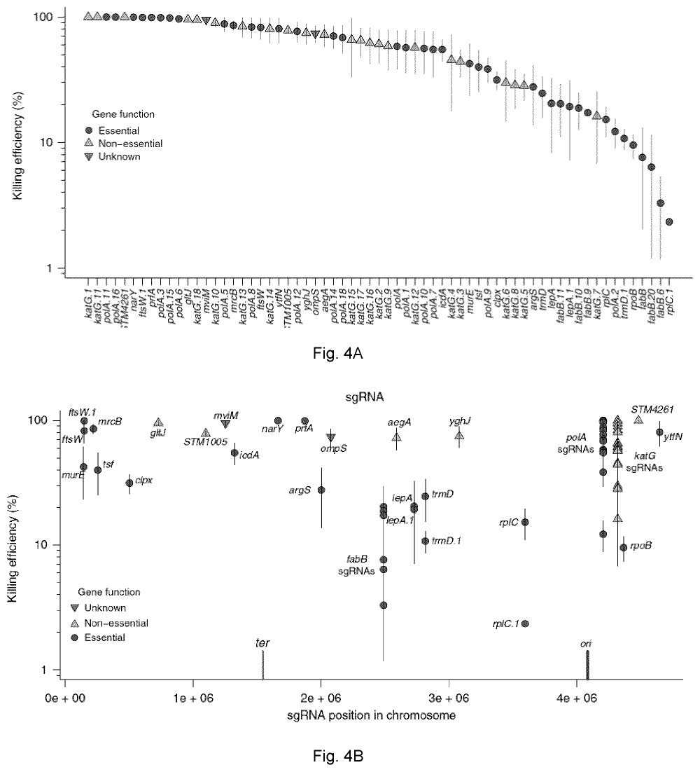

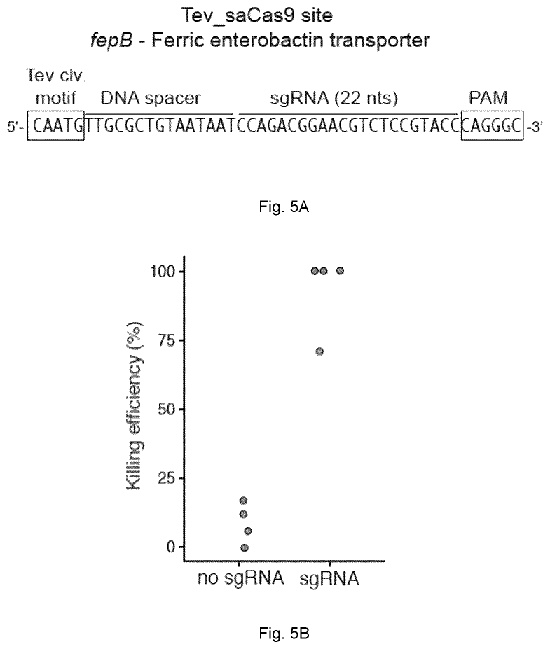

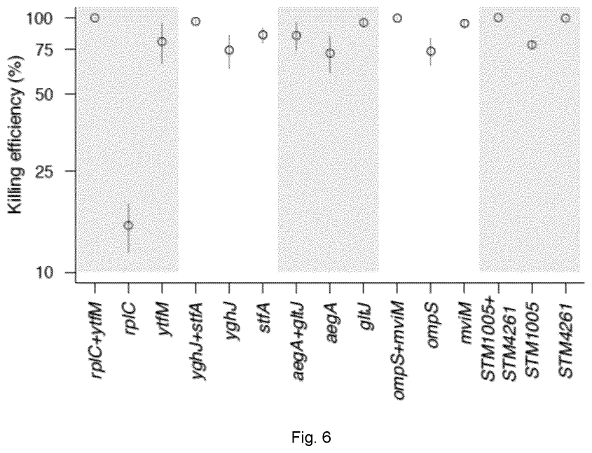

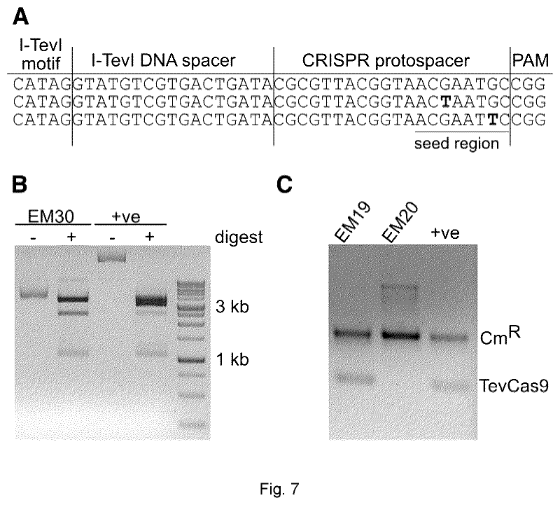

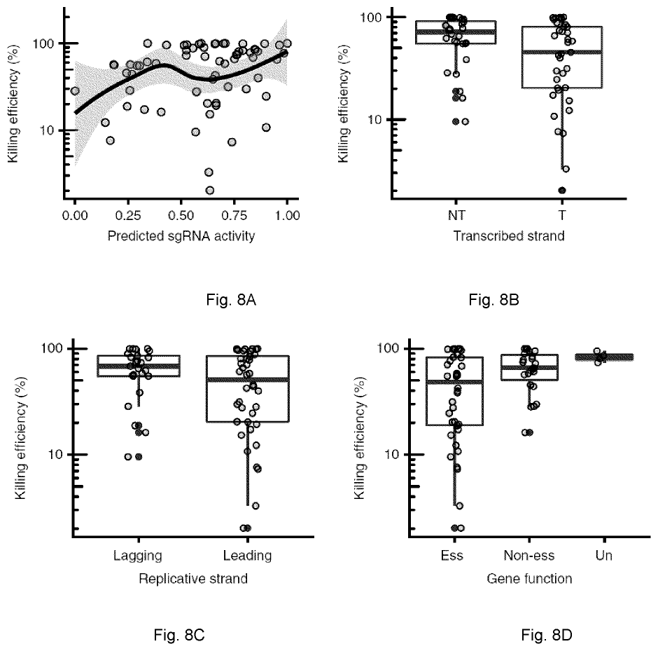

To demonstrate that the TevSpCas9 nuclease could be delivered by conjugation to eliminate specific bacterial species, we designed 65 total sgRNAs targeting 38 essential genes, 23 nonessential genes, and 4 genes with unresolved phenotypes (FIG. 4A and Table 2. The 65 sgRNA sites were arrayed around the S. enterica chromosome (FIG. 4B, differed in their relative position within each gene, and what strand was being targeted. We assessed the efficacy of each sgRNA in killing S. enterica by comparing the ratio of S. enterica colony counts under conditions where TevSpCas9 expression from the pBAD promoter was induced with arabinose or repressed with glucose. Using E. coli as the conjugative donor, we found a range of S. enterica killing efficiencies between 1 and 100% (FIG. 4A). To demonstrate that the I-Tevl nuclease domain could function in the context of other Cas9 orthologs, we fused the I-Tevl nuclease domain to SaCas9 from Staphylococcus aureus to create TevSaCas9. SaCas9 differs from SpCas9 in possessing a longer PAM requirement. With TevSaCas9 we observed high killing efficiency (93±8%, mean±standard error) when TevSaCas9 was targeted to the fepB gene of S. enterica (FIGS. 5A and B). sgRNAs expressed as pairs from separate promoters also yielded high killing efficiencies (FIG. 6), demonstrating the potential for multiplexing guides to overcome mutational inactivation of individual guides. Sampling S. enterica colonies resistant to killing from experiments with different sgRNAs revealed three types of escape mutants: nucleotide polymorphisms in the chromosome target site that would weaken sgRNA-DNA interactions, transposable element insertions that inactivated sgRNA expression, and rearrangements of pNuc that impacted TevSpCas9 function (FIG. 7A-7C).

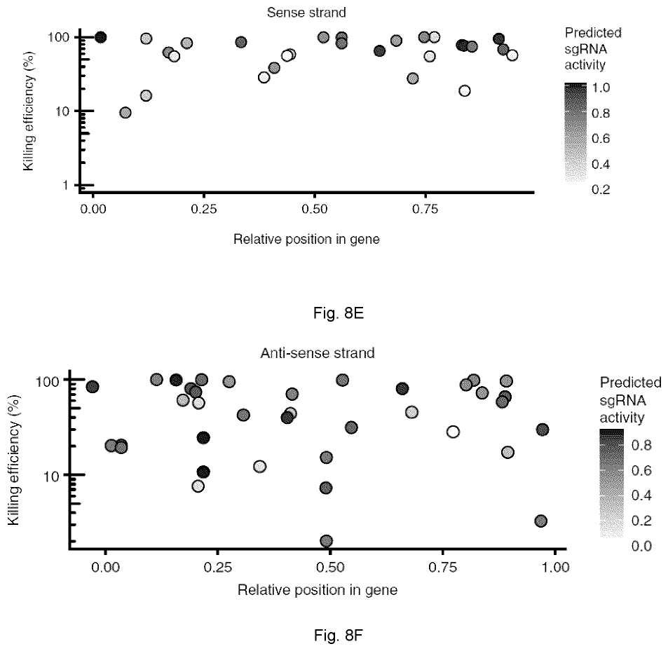

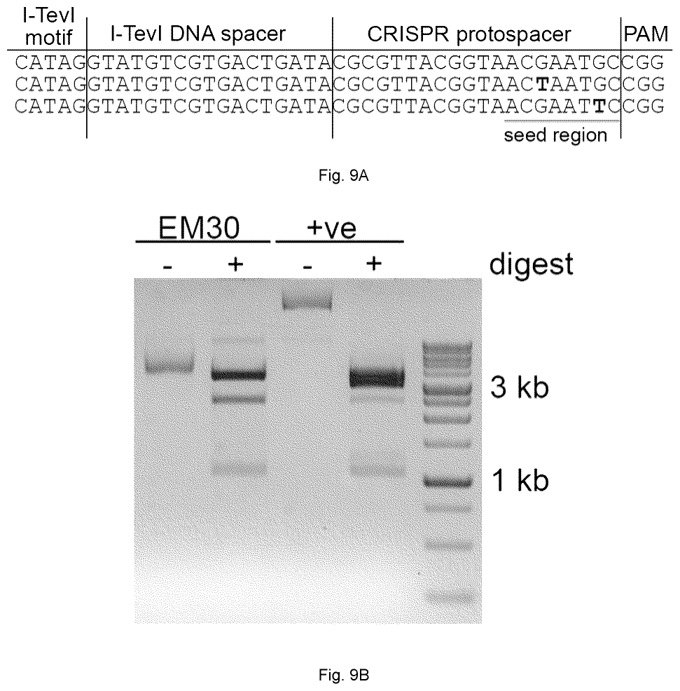

We considered a number of variables that would influence sgRNA killing efficiency in S. enterica, including predicted sgRNA activity according to an optimized prokaryotic model41, targeting of the sense or anti-sense strands for transcription, the relative position of the sgRNA in the targeted gene, targeting of the leading or lagging replicative strands, and the essentiality of the targeted gene. Taken independently, no single variable was strongly correlated with sgRNA killing efficiency (FIG. 8 and FIG. 9). A generalized linear model was used to assess the significance of each variable on sgRNA killing efficiency, revealing that sgRNA score positively correlated with predicted activity (p<0.02, t test) while targeting essential genes was negatively correlated with killing efficiency (p<0.03, t test) (FIG. 10). The moderate statistical support from the linear model suggests that a robust understanding of parameters that influence sgRNA targeting and activity in prokaryotic genomes remains a work in progress, particularly in the context of conjugative plasmids.

During the course of these experiments, we noted that some sgRNAs were recalcitrant to cloning (FIG. 11). In particular, sgRNAs targeting essential genes in S. enterica were more likely to yield inactive clones than sgRNAs targeting nonessential genes (Table 5). Whole plasmid sequencing revealed no insertions in 15 clones with sgRNAs targeting nonessential genes, whereas 7/13 clones sgRNAs targeting essential genes had insertions. These findings suggest that leaky expression of the TevSpCas9 nuclease from the pBAD promoter is sufficient to cause cellular toxicity in E. coli, and selection for inactive plasmids. Thus, choosing sgRNAs with minimal identity and off-target sites in the E. coli genome will facilitate conjugative delivery of sgRNAs and CRISPR nucleases.

This study shows an IncP RK2 conjugative plasmid to function as a delivery system. This study differs from previous attempts to use conjugation as a delivery system in one key facet—a cis setup where the pNuc plasmid encoded the conjugation machinery as well as the TevCas9 nuclease. The pNuc-cis plasmid of this invention promotes efficient conjugation because ex-conjugants become donors for subsequent re-conjugation, leading to significant increases in conjugation relative to the pNuc-trans plasmid (see FIG. 1C).

Others have employed strains with the conjugation machinery embedded in the chromosome of the donor bacteria (similar to the pNuc-trans setup), meaning that only a single round of conjugation could occur. In the two-species E. coli-S. enterica used in this study system, it was observed conjugation efficiencies approaching ˜100% with pNuc-cis in culture conditions that promoted cell-to-cell contact and biofilm formation. Because the IncP RK2 system can be conjugated to a wide diversity of bacteria, the cis-conjugation system of the present invention could be used to deliver the TevCas9 nuclease (or other CRISPR nuclease) in complex microbial communities. Anti-CRISPR proteins that are specific for relevant CRISPR systems could also be included on pNUC-cis to prevent acquisition of CRISPR-mediated resistance.

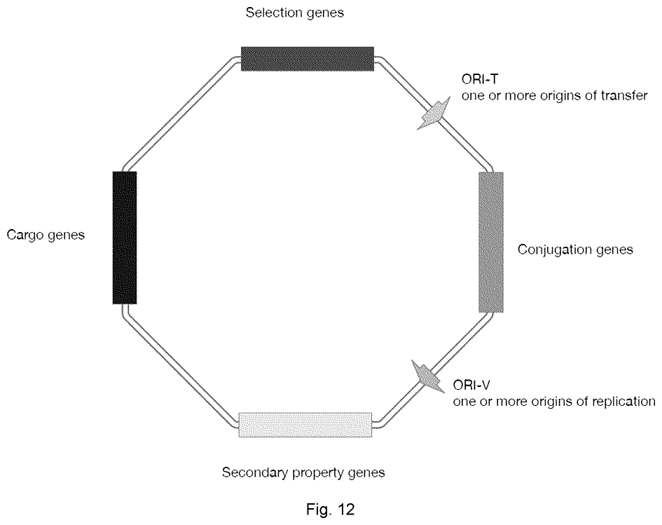

Microbiomes could also be seeded with multiple strains of donor bacteria harbouring versions of pNUC-cis based on different conjugative plasmid backbones (FIG. 12), each encoding redundant programmable CRISPR nucleases or other anti-microbial agents.

Microbial communities are complex in terms of bacterial composition and the environments they inhabit. Many human microbial communities exist as biofilms, which presents challenges for delivery of anti-microbial agents. Indeed, a number of disease conditions result from microbial imbalances in mucosal surfaces that are dominated by biofilms. Conjugative plasmids express factors to promote biofilm formation to enhance cell-to-cell contact necessary for formation of the conjugative pilus. By using a donor bacteria that is a native resident of the target microbiome the pNUC-cis plasmid could be introduced to microbial communities more readily than delivery vectors that have difficulty penetrating biofilms. Conversely, other delivery vectors, such as phage-based methods, are better suited to planktonic conditions where conjugation is less efficient.

Depending on the nature of the microbiome and dysbiosis, a combination of conjugative- and phage-based CRISPR delivery systems may also be used.

TABLE 1
Primers used to construct plasmids

| NAME | SEQUENCE (5′-3′) | NOTES |
|---|---|---|
| DE-2031 | GGGCGTTGGAATCCAGAAACC | Forward primer to amplify |
| | | TevCas9 fragment from |
| | | within the I-Tevl domain |
| DE-3116 | TTACGCCCCGCCCTGCCACT | Reverse primer to amplify |
| | | chloramphenicol resistance |
| | | gene fragment |
| DE-3302 | GGCATCGGTCGAGATCCCGGTGCCTAATGAGTGAGC | Forward primer to amplify |
| | TAACTTACATTAATTGCGTTGCGCGATCGTCTTGCC | OriT fragment with overlap |
| | TTGCTCGT | to pACYC backbone fragment |
| | | to clone pNuc-trans |
| DE-3303 | GTAGCATAGGGTTTGCAGAATCCCTGCTTCGTCCAT | Reverse primer to amplify |
| | TTGACAGGCACATTATGCATCGATATCTTCCGCTGC | OriT fragment with overlap |
| | ATAACCCT | to AraC/pBad fragment to |
| | | clone pNuc-trans |
| DE-3304 | GATGGATATACCGAAAAAATCGCTATAATGACCCCG | Forward primer to amplify |
| | AAGCAGGGTTATGCAGCGGAAGATATCGATGCATAA | AraC/pBAD fragment with |
| | TGTGCCTG | overlap to OriT fragment |
| | | to clone pNuc-trans |
| DE-3305 | CCATGGTATATCTCCTTATTAAAGTTAAACAAAATT | Reverse primer to amplify |
| | ATTTCTACAGGGCTAGCCCAAAAAAACGGG | AraC/pBAD fragment with |
| | | overlap to TevCas9 fragment |
| | | to clone pNuc-trans |
| DE-3306 | GACGCTTTTTATCGCAACTCTCTACTGTTTCTCCAT | Forward primer to amplify |
| | ACCCGTTTTTTTGGGCTAGCCCTGTAGAAATAATTT | TevCas9 with overlap to |
| | TGTTTAAC | AraC/pBad fragment to clone |
| | | pNuc-trans |
| DE-3307 | TCTCCCGTGCTCAGTATCTCTATCACTGATAGGGAT | Reverse primer to amplify |
| | GTCAATCTCTATCACTGATAGGGAATTTCGATTATG | TevCas9 with overlap to the |
| | CGGCCGTG | gRNA cassette to clone |
| | | pNuc-trans |
| DE-3308 | CGAAATTCCCTATCAGTGATAGAGATTGACATCCCT | Forward primer to amplify |
| | ATCAGTGATAGAGATACTGAGCACGGGAGACCCATG | gRNA cassette with overlap |
| | CCATAGCG | to TevCas9 fragment to |
| | | clone pNuc-trans |
| DE-3309 | GCTCCATCAAGAAGAGGCACTTCGAGCTGTAAGTAC | Reverse primer to amplify |
| | ATCACCGACGAGCAAGGCAAGACGATCGCGCAACGC | pACYC backbone with |
| | AATTAATG | overlap to OriT fragment to |
| | | clone pNuc-trans |
| DE-3315 | TTTATATATTTATATTAAAAAATTTAAATTATAATT | Reverse primer to amplify |
| | ATTTTTATAGCACGTGATGCTCGCCAAAAAACCCCT | gRNA cassette with overlap |
| | CAAGACCC | to CEN-ARS-HIS fragment to |
| | | clone pNuc-trans |
| DE-3316 | GCTCCGCTGAGCAATAACTAGCATAACCCCTTGGGG | Forward primer to amplify |
| | CCTCTAAACGGGTCTTGAGGGGTTTTTTGGCGAGCA | CEN-ARS-HIS with overlap |
| | TCACGTGC | to gRNA cassette to clone |
| | | pNuc-trans |
| DE-3351 | TATTGACTACCGGAAGCAGTGTGACCGTGTGCTTCT | Reverse primer to amplify |
| | CAAATGCCTGAGGTTTCAGTCAAGTCCAGACTCCTG | CEN-ARS-HIS with overlap |
| | TGTAAAAC | to pACYC backbone (p15A |
| | | origin and CAT gene) to |
| | | clone pNuc-trans |
| DE-3352 | ACGATGTTCCCTCCACCAAAGGTGTTCTTATGTAGT | Forward primer to amplify |
| | TTTACACAGGAGTCTGGACTTGACTGAAACCTCAGG | pACYC backbone with |
| | CATTTGAG | overlap to CEN-ARS-HIS |
| | | fragment to clone pNuc-trans |
| DE-3365 | CACGCGCGTTACGGTAACGAATGCG | Top strand oligo to clone |
| | | sgRNA 9 targeting STM1005 |
| DE-3366 | AAAACGCATTCGTTACCGTAACGCG | Bottom strand oligo to clone |
| | | sgRNA 9 targeting STM1005 |
| DE-3367 | CACGCCAGGGAATACGTGGGCGGAG | Top strand oligo to clone |
| | | sgRNA 10 targeting |
| | | STM4261 |
| DE-3368 | AAAACTCCGCCCACGTATTCCCTGG | Bottom strand oligo to clone |
| | | sgRNA 10 targeting |
| | | STM4261 |
| DE-3424 | GAATTTCTGCCATTCATCCGCTTATTATCACTTATT | Forward primer to amplify |
| | CAGGCGTAGCACCAGGCGTTTAACGATCGTCTTGCC | pNuc-trans with overlap to |
| | TTGCTCGT | pTA-mob Avrll site to clone |
| | | pNuc-cis |
| DE-3425 | GCGTCCTGCTCGTGATCGGGAGTATCTGGCTGGGCC | Reverse primer to amplify |
| | AACGTTCCAACCGCACTCCTAGTCAAGTCCAGACTC | pNuc-trans with overlap to |
| | CTGTGTAA | pTA-mob Avrll site to clone |
| | | pNuc-cis |
| DE-3537 | GAGGGCACCGATAAGATTCTT | Reverse primer to amplify |
| | | TevCas9 gene fragment from |
| | | within Cas9 domain |
| DE-3748 | CCTGGTTGAGCAGAGAAACCT | Forward primer to amplify |
| | | STM1005 target site from |
| | | Salmonella genomic DNA |
| DE-3749 | GTTGCGGGAATATGGACAAT | Reverse primer to amplify |
| | | STM1005 target site from |
| | | Salmonella genomic DNA |
| DE-3750 | CTGCTTTCTAAGGATGATACGG | Forward primer to amplify |
| | | STM4261 target site from |
| | | Salmonella genomic DNA |
| DE-3751 | TTATCGCCTTTCACGCC | Reverse primer to amplify |
| | | STM4261 target site from |
| | | Salmonella genomic DNA |
| DE-3752 | GTCCGAATAGCGCTAATAGCATATCATACGGCGAGC | Forward primer to amplify |
| | ATCACGTGCTATAA | backbone and initial sgRNA |
| | | (overhang A) for |
| | | multiplexing sgRNAs |
| DE-3753 | CGTATGATATGCTATTAGCGCTATTCGGACCAAAAA | Reverse primer to amplify |
| | ACCCCTCAAGACCC | second sgRNA to 5′ end of |
| | | backbone (overhang A) for |
| | | multiplexing sgRNAs |
| DE-3754 | ACCGTTAGCATCGATCTACACATTAGGACAGTATTG | Forward primer to amplify |
| | TACACGGCCGCATA | second sgRNA cassette |
| | | (overhang B) for |
| | | multiplexing sgRNAs |
| DE-3755 | TGTCCTAATGTGTAGATCGATGCTAACGGTCAAAAA | Reverse primer to amplify |
| | ACCCCTCAAGACCC | backbone with overhang to |
| | | second sgRNA cassette |
| | | (overhang B) for |
| | | multiplexing sgRNAs |
| DE-3777 | ATGGAGAAAAAAATCACTGGATATAC | Forward primer to amplify |
| | | chloramphenicol resistance |
| | | gene fragment |
| DE-4188 | AATGCCGTGTTTATCTCGTCAACTTGTTGGCGAGAT | Forward primer to amplify |
| | TTTTTCCGCTGAGCAATAACTAGC | saCas9 with homololgy to |
| | | I-Tevl linker in pNuc |
| | | construct |
| DE-4189 | CCAGGATGTAGTTCCGCTTGGCTGCTGGGACTCCGT | Reverse primer to amplify |
| | GGATACCGCTACCTCCGGTACCAC | saCas9 with homology to |
| | | gRNA cassette in pNuc |
| | | construct |
| DE-4255 | CACGCCAGACGGAACGTCTCCGTACC | Forward primer to amplify |
| | | pNuc backbone with |
| | | homology to the RNA |
| | | cassette |
| DE-4256 | AAACGGTACGGAGACGTTCCGTCTGG | Reverse primer to amplify |
| | | pNuc with Tev backbone |
| | | with homology to saCas9 |

TABLE 2

| Target Site (SEQ ID NO: #) | Target Sequence | Notes |
|---|---|---|
| Target Site 1 (SEQ ID NO: 1) | gttaaaaaagttgacgtaac | Targets in the rpIC gene at position 3595884 |
| | | in S. enterica LT2 genome |
| Target Site 2 (SEQ ID NO: 2) | gttaaaaaagttgacgtaac | Targets in the rpIC.1 gene at position 3595884 |
| | | in S. enterica LT2 genome |
| Target Site 3 (SEQ ID NO: 3) | ctgaatatcgagtcatttcg | Targets in the ytfN gene at position 4648516 |
| | | in S. enterica LT2 genome |
| Target Site 4 (SEQ ID NO: 4) | gttgatcggttcataaaacg | Targets in the yghJ gene at position 3081003 |
| | | in S. enterica LT2 genome |
| Target Site 5 (SEQ ID NO: 5) | acgccagtatgatctttcgc | Targets in the mrcB gene at position 221766 |
| | | in S. enterica LT2 genome |
| Target Site 6 (SEQ ID NO: 6) | acgcggcttggcgaaccgga | Targets in the aegA gene at position 2589934 |
| | | in S. enterica LT2 genome |
| Target Site 7 (SEQ ID NO: 7) | ccatagccagccgagatagg | Targets in the gltJ gene at position 728675 |
| | | in S. enterica LT2 genome |
| Target Site 8 (SEQ ID NO: 8) | attaaggtaaacaccaccga | Targets in the ompS gene at position 2077806 |
| | | in S. enterica LT2 genome |
| Target Site 9 (SEQ ID NO: 9) | tgccggcgtccatgtctgcg | Targets in the mviM gene at position 1254019 |
| | | in S. enterica LT2 genome |
| Target Site 10 (SEQ ID NO: 10) | cgcgttacggtaacgaatgc | Targets in the STM1005 gene at position |
| | | 1098447 in S. enterica LT2 genome |
| Target Site 11 (SEQ ID NO: 11) | ccagggaatacgtgggcgga | Targets in the STM4261 gene at position |
| | | 4486054 in S. enterica LT2 genome |
| Target Site 12 (SEQ ID NO: 12) | aggcagtggccgacgccggtc | Targets in the fabB gene at position 2489593 |
| | | in S. enterica LT2 genome |
| Target Site 13 (SEQ ID NO: 13) | gatcccgacggagaacacaac | Targets in the murE gene at position 143935 |
| | | in S. enterica LT2 genome |
| Target Site 14 (SEQ ID NO: 14) | tcgaagaagagcgcgttgctc | Targets in the tsf gene at position 255625 |
| | | in S. enterica LT2 genome |
| Target Site 15 (SEQ ID NO: 15) | cgagatgcccatcccgataa | Targets in the ftsW gene at position 149408 |
| | | in S. enterica LT2 genome |
| Target Site 16 (SEQ ID NO: 16) | cgagatgcccatcccgataa | Targets in the ftsW gene at position 149408 |
| | | in S. enterica LT2 genome |
| Target Site 17 (SEQ ID NO: 17) | tacgcgcagcggtgcggaat | Targets in the rpoB gene at position 4366214 |
| | | in S. enterica LT2 genome |
| Target Site 18 (SEQ ID NO: 18) | aggggcgccgcctttacctgc | Targets in the polA gene at position 4208600 |
| | | in S. enterica LT2 genome |
| Target Site 19 (SEQ ID NO: 19) | aacctgagccgccagggcat | Targets in the icdA gene at position 1325325 |
| | | in S. enterica LT2 genome |
| Target Site 20 (SEQ ID NO: 20) | ataacgaatgcgcccgacgc | Targets in the narY gene at position 1665221 |
| | | in S. enterica LT2 genome |
| Target Site 21 (SEQ ID NO: 21) | atccgcagcaggagttcttac | Targets in the clpx gene at position 504775 |
| | | in S. enterica LT2 genome |
| Target Site 22 (SEQ ID NO: 22) | gctcgtcagccggcatatcc | Targets in the argS gene at position 2003842 |
| | | in S. enterica LT2 genome |
| Target Site 23 (SEQ ID NO: 23) | ggcggaccggggatgttaatga | Targets in the trmD gene at position 2815864 |
| | | in S. enterica LT2 genome |
| Target Site 24 (SEQ ID NO: 24) | ggcggaccggggatgttaatga | Targets in the trmD gene at position 2815864 |
| | | in S. enterica LT2 genome |
| Target Site 25 (SEQ ID NO: 25) | aggttcaggacgatatcgaga | Targets in the prfA gene at position 1874237 |
| | | in S. enterica LT2 genome |
| Target Site 26 (SEQ ID NO: 26) | tgaccgtattatccaaatctg | Targets in the lepA gene at position 2728509 |
| | | in S. enterica LT2 genome |
| Target Site 27 (SEQ ID NO: 27) | tgaccgtattatccaaatctg | Targets in the lepA gene at position 2728509 |
| | | in S. enterica LT2 genome |
| Target Site 28 (SEQ ID NO: 28) | tattccgggcgtaccaggcg | Targets in the polA gene at position 4206710 |
| | | in S. enterica LT2 genome |
| Target Site 29 (SEQ ID NO: 29) | atcgcccagcgaaccggcag | Targets in the polA gene at position 4207091 |
| | | in S. enterica LT2 genome |
| Target Site 30 (SEQ ID NO: 30) | agatcgcactggaggaagcg | Targets in the polA gene at position 4207606 |
| | | in S. enterica LT2 genome |
| Target Site 31 (SEQ ID NO: 31) | gccgctggatagcgtgaccg | Targets in the polA gene at position 4208375 |
| | | in S. enterica LT2 genome |
| Target Site 32 (SEQ ID NO: 32) | ttaaatccagcaacgcggcg | Targets in the polA gene at position 4208626 |
| | | in S. enterica LT2 genome |
| Target Site 33 (SEQ ID NO: 33) | taacgacttcatccgggccg | Targets in the polA gene at position 4206642 |
| | | in S. enterica LT2 genome |
| Target Site 34 (SEQ ID NO: 34) | tacgcccggaatattatccg | Targets in the polA gene at position 4206722 |
| | | in S. enterica LT2 genome |
| Target Site 35 (SEQ ID NO: 35) | caggttcgatggcaaacgag | Targets in the polA gene at position 4207275 |
| | | in S. enterica LT2 genome |
| Target Site 36 (SEQ ID NO: 36) | gcagttccagagcacgctgg | Targets in the polA gene at position 4207356 |
| | | in S. enterica LT2 genome |
| Target Site 37 (SEQ ID NO: 37) | taaatgcctgacgaatgcgg | Targets in the polA gene at position 4208223 |
| | | in S. enterica LT2 genome |
| Target Site 38 (SEQ ID NO: 38) | aagctggcgagaaagaccga | Targets in the polA gene at position 4208474 |
| | | in S. enterica LT2 genome |
| Target Site 39 (SEQ ID NO: 39) | acctgtcgcgcatgattatc | Targets in the polA gene at position 4207292 |
| | | in S. enterica LT2 genome |
| Target Site 40 (SEQ ID NO: 40) | ttaactttggcctgatttac | Targets in the polA gene at position 4208422 |
| | | in S. enterica LT2 genome |
| Target Site 41 (SEQ ID NO: 41) | cgagaataagtgggttttct | Targets in the polA gene at position 4206177 |
| | | in S. enterica LT2 genome |
| Target Site 42 (SEQ ID NO: 42) | catggcgcgcttgatgatat | Targets in the polA gene at position 4208723 |
| | | in S. enterica LT2 genome |
| Target Site 43 (SEQ ID NO: 43) | gtggccgaaccagcttcgcg | Targets in the katG gene at position 4319703 |
| | | in S. enterica LT2 genome |
| Target Site 44 (SEQ ID NO: 44) | tgaccgattcacaaccgtgg | Targets in the katG gene at position 4319840 |
| | | in S. enterica LT2 genome |
| Target Site 45 (SEQ ID NO: 45) | cctcggtaaaacccacggcg | Targets in the katG gene at position 4320399 |
| | | in S. enterica LT2 genome |
| Target Site 46 (SEQ ID NO: 46) | cgcggcggcgataagcgtgg | Targets in the katG gene at position 4321030 |
| | | in S. enterica LT2 genome |
| Target Site 47 (SEQ ID NO: 47) | accttttgcgccgggccggg | Targets in the katG gene at position 4321245 |
| | | in S. enterica LT2 genome |
| Target Site 48 (SEQ ID NO: 48) | gtttgtgaaggacttcgtcg | Targets in the katG gene at position 4321710 |
| | | in S. enterica LT2 genome |
| Target Site 49 (SEQ ID NO: 49) | gctggttcggccaccagtcg | Targets in the katG gene at position 4319716 |
| | | in S. enterica LT2 genome |
| Target Site 50 (SEQ ID NO: 50) | ggtagcgcgaatagcggcgg | Targets in the katG gene at position 4320339 |
| | | in S. enterica LT2 genome |
| Target Site 51 (SEQ ID NO: 51) | gccctgcgcttcaatcggcg | Targets in the katG gene at position 4320477 |
| | | in S. enterica LT2 genome |
| Target Site 52 (SEQ ID NO: 52) | gccgccgcggaaagtagacg | Targets in the katG gene at position 4321038 |
| | | in S. enterica LT2 genome |
| Target Site 53 (SEQ ID NO: 53) | gatgctgacacccgcagcag | Targets in the katG gene at position 4321239 |
| | | in S. enterica LT2 genome |
| Target Site 54 (SEQ ID NO: 54) | aaccaaacaccagatcggcg | Targets in the katG gene at position 4321651 |
| | | in S. enterica LT2 genome |
| Target Site 55 (SEQ ID NO: 55) | caactatatctatttgctcc | Targets in the katG gene at position 4319533 |
| | | in S. enterica LT2 genome |
| Target Site 56 (SEQ ID NO: 56) | ttctattagcgagatggttt | Targets in the katG gene at position 4320981 |
| | | in S. enterica LT2 genome |
| Target Site 57 (SEQ ID NO: 57) | tgacttcttcgctaatctgc | Targets in the katG gene at position 4321515 |
| | | in S. enterica LT2 genome |
| Target Site 58 (SEQ ID NO: 58) | cgccttgagatcccctttca | Targets in the katG gene at position 4319835 |
| | | in S. enterica LT2 genome |
| Target Site 59 (SEQ ID NO: 59) | ttgataatgtcttcctgcgt | Targets in the katG gene at position 4320950 |
| | | in S. enterica LT2 genome |
| Target Site 60 (SEQ ID NO: 60) | agctcattagcgtcgtcggt | Targets in the katG gene at position 4321580 |
| | | in S. enterica LT2 genome |
| Target Site 61 (SEQ ID NO: 61) | tggcggcaccaacgccacgc | Targets in the fabB gene at position 2488660 |
| | | in S. enterica LT2 genome |
| Target Site 62 (SEQ ID NO: 62) | agagctggatgagcaggctg | Targets in the fabB gene at position 2488750 |
| | | in S. enterica LT2 genome |
| Target Site 63 (SEQ ID NO: 63) | cgccagccgcgcccagcgag | Targets in the fabB gene at position 2488818 |
| | | in S. enterica LT2 genome |
| Target Site 64 (SEQ ID NO: 64) | cgtgcagtgattactggcct | Targets in the fabB gene at position 2489829 |
| | | in S. enterica LT2 genome |
| Target Site 65 (SEQ ID NO: 65) | ggcctgtgagttcgatgcga | Targets in the fabB gene at position 2489245 |
| | | in S. enterica LT2 genome |

TABLE 3
Sequence of pNuc-Cis (SEQ ID NO: 66)
ttcacccccgaacacgagcacggcacccgcgaccactatgccaagaatgcccaaggtaaaaattgccggccccgccatgaagtccgtga
atgccccgacggccgaagtgaagggcaggccgccacccaggccgccgccctcactgcccggcacctggtcgctgaatgtcgatgccagc
acctgcggcacgtcaatgcttccgggcgtcgcgctcgggctgatcgcccatcccgttactgccccgatcccggcaatggcaaggactgc
cagcgccgcgatgaggaagcgggtgccccgcttcttcatcttcgcgcctcgggcctcgaggccgcctacctgggcgaaaacatcggtgt
ttgtggcattcatacggactcctgttgggccagctcgcgcacgggctggcgggtcagcttggcttgaagatcgccacgcattgcggcga
tctgcttctcggcatccttgcgcttctgcacgccttcctgctggatgcgaataacgtcctcgacggtcttgatgagcgtcgtctgaacc
tgcttgagcgtgtccacgtcgatcaccaggcgttggttctccttcgccgtctcgacggacgtgcgatgcagcagggccgcattgcgctt
catcaggtcgttggtggtgtcgtcgatggccgtggccagttcgacggcgttcttctgctcgttgaggctcaaggccagcatgaattgcc
gcttccacgccggcacggtgatttcgcggatggtgtggaatttatcgaccagcatctggttgttggcctggatcatgcggatggtcggc
aggctctgcatggccgaatgttgcaaggcgatcaggtcgccgatgcgcttgtccaggttggcaaccatcgcatcgaggtcggccagctc
ctgcacgcggcccgggtcgttcccgacattgccgcgcagaccctcggcctgctcgcgcagctcggcaaggcggaccttgccggccgcga
tgtggacgccaagaaggcggtgttcctcgcgcacggctgcgaacatttcgtcgagcgaggcattgcgctgcgcgatgccttgctgggtg
gtctgcacttcgctgaccaggtgttcgatctgctcgcgggtcgtgtcgaagcgcgccatgaagcccgtcgaacggacgcggaagcggtc
gatcagcgggccaatcaggggcaggcgggaacggttgtcggacaaagggccgacgttcagggaacgggccttggcgacaacctgggtca
gtttctcgcctgcttcgtccaggtcgctgttgcgcacctggtccagcaggctatcggcgtagcgggacgtgtgctcggccacgtcgcgg
ccgaactcggcaacggtctgcggactgccgacctcgatccgctgcgcgaccgcatggacttccggcacgtcgctttcctgcaagcccag
ctcgcgcagggttgccggggtcatgtcgaaggcgacgataggggccttggcgtcgtgcgtcgttttcagtgcgttcatagggttctccc
gccgtgttattggttgatgccttccaggctctgcgaaaggctccgcatgagcgcctggtgagctttggccgcctcggcgaccattgccg
gattcatgttcttggtggtgatgagcgcgagggtgtgctgacgccagacgggcaccaggacggatgccgtttcagagaagcggtccagc
atgtccacggcctgcgcccgcgtgagcttcatctgagtgacgctcatttcatgggacgccatgagggttgccaggttggcgagcttgcg
cgcgaagcgttcgcgcggcttgtcgaactcgatcacgccggccttggccgcgccggcctcggggttctcgtccaggaactcgcgcccgg
cttgaatgtaggctctgagccggtctacctcggcctcatgcgtattgagcatgtcatccaaggcgcgcaacgtgtcccgcacgcgctgc
gctacgccctcggcttcgtccagcaactggtcgagcgtcttgcgggcgacctgatacctcacctggcgttcaacctcacggccaagcat
cttctcgaaccaggtaggcttttccgcgatcttgcgggggtccgcgtcggccagcttcgccacgatctggctgattttgtcggccagcg
cggcaactgcgccgtgctccatcagattcgacagctcgttgagggaatccgccccgtcgatgccggccccgtactcgccaatcgtcgcc
ggcgacgcgaagagggcgggcaaaacctcccccttcaatcgcgccatgttcacgctttgttcttccattcgatacaccctcgcggtggg
ttaattgcttttcgatggaagaagtttagctaaactttctatccctcgtcaacacctttagccgctaaaatttggggacaggtcattta
cagaaagccagctcactcctggcgttgccccttgagcgccgctaggcgcgcagcatccttcgcgctgagaaagaacgtcatcagcggcc
cgaccgtcttgcttgaaccgtcggcaaagcaaacatccatcgaacagccttgcgtgtgggggtccacgccttcgaccagtttccaaggg
tccatgccccagccctcggcctccggattgaaccagtacgccgatgcgtcgccgtttaggtcgctgtcggcgtagtccttgaccagcac
ggccacccgcccggtcgtcgggcacacgtagcccggctgcttaggttcctgtcttggcattgctcaaagctccttgaaggggccgctct
acagccccttgggcttgtagagcgacacgaaataggtgagtgcggtcagtaccgcgaaatgcaccaggaacgtccagccggcatgaacg
ccaagggtgttccagtggtacagcatccgcaggaactgaaagaaaacgtcgatagagatgatccacttcgccaccggccacaccaggac
agtaacgacccatacaaagcggaccagggcctggacacccttggcaaaagtgaaccggggcggggccttgctcggggcctcaacgcgcg
gggcaggggcctcggcctccacttccacgcctgggaacttgataatcttcgacattgcttgaccctccacggcgatgcgtgttcaattc
gtccagcgctcgcgcgcctagaccgtgatgtgacagcatcgaggtcaagcgccccggagaaatccggggcgtcatccctatgccccgtc
caactcgggaaccggcttttccctggtgctcaacctggccggctcgacccacttggtgacttgctgccactcgttaccactgcgaacgg
ctacccgaatctgcacacgtttagccgcgatcttcgtcactatgccagcaacacaaacggaatacccgtacccgccgcgcggggtgtgc
tgccagttcaccctatctcctacttgcatcatcatcccctggcgtcagtgaccggcccggaatttcgccaagtcgatttcgttgaaggt
ccagcgctgtttcgcccgttcctccaacctcgacgactcccgcatgacctcgatgcgcaggcgctcgacctggtgcatcagctcatcag
cgcgccgctgcttctcggcaatagcattccgctgcgcgaccagctcctggtctacgttcggcagctcgtcgatccacggcatgaactta
tcggtcatcggattggcctccggtaattgacctgggaatctacccggcctcaaaacaagaatagggcataatgccctaacttgtcaagc
aattttagctaaacaattgaggggattcagcgaggcgtcatgcttgaaaacacctttcccctggcgtgcaatcagcttgtcccggcggc
agcgcactgccgcagcgcggccagcaaggtgccttgctcgatccgctgtcgttcctccggcgtaatgcaatcgccgcagataccgccca
gctcttcgcgcagggcgtccgccccgatcaggctgcggctaagggtcgaaatggacttaccgcagcggcggcaattggtggtgacaatc
tcgatcttgcggttgggcatggccctatctccttgagagaggcccgaccgtagccgggcctcgttccgttaccagcctgcgcgagcctt
cgcgcgttcgacttcctgggtggaccagtggccctttgcttcaccctccagggtgcaaccttgcaggtacgccttgtgccgcagctctt
cggcctggcgggtaagctctgccgcctgctccatcagcgccgcgatctggtcacgacgggccagaaaatccgtggcggccgcgccgggc
agctcatcgagccaagacatgatcttgcttttcatcggggttgatcctccggttgctgacctgggcgaagtgcccggccttggattgct
atattagggcattatgccctagatagtcaaggaaatttagctaaacaatttgcggcgggcgcacgaaaaaacccggcttgcgggccggg
ctgctggcagcatatcgcaacgatcagggttggcggttttagccgctaaagtcctctcccttggcgtaaagtcctgcgggcgtcagccc
tggcctttccagatcgccccaatccccgctagatcgcaaaggatcgcccaggcggcataggggatcggcgaatcctcgccaacccaacg
ccgcaccgtgcggtcgcccttggcacccaagcccaagatgcgcgcagcctgtccgccggtgaggccggccaagtgcaagacttcccgga
tttctgcgccggtcggctgcacccagcgttccgccgggcgcaggcactcaagccggatattcacgtcgctcatgctgcttttctcctaa
tcgttatcaaatggcggcccgcaattggtcgtagccgtagcacgactcgatgcaacgcgggtcatattcacaaacctttctaccgttgc
gattgatgcgatagcgcggcggcttgtcgtcgtgcgcccacacctgtcgcgtttcgggactgatcttcgtaatgatgacgtgcttcccg
atttccttggcaaaaaccgggtgatgcacgctcacaatctcgcaccgcaggccaggaaagaacgtcgccggccacgccggcgcgtcctc
gtccttggccggctccggctccggcttggccggtgtaaccggctccctgcgctgcccggccggctcctgagctttcgcacgcgcggccg
cgagcttcgccttggcgctggccgcccatttgcgggcgatgaatgcctggtgcgccggcagcacggcatcaacccaggcgaagcccggc
ggcagttcgtccggcttccgccagtcctcgaccacggcccgcacgcggcgcggcggtggcgtgatacccgcgagccacgccggcagtgg
ctcgtcggcaaggcggtcctggtcgcgggcgtccagataccggcgatgcgagcgcaacagggcgcgcaaaaccccgtccgctacctggt
ccaccgtcatgccgccgcgcgcatggtcgtaatgggaccgatagcccgtttcggaaataaaaggggtctgacgctcagtggaacgaaaa
ctcacgttaagggattttggtcatgagattatcaaaaaggatcttcacctagatccttttaaattaaaaatgaagttttaaatcaatct
aaagtatatatgagtaaacttggtctgacagttaccaatgcttaatcagtgaggcacctatctcagcgatctgtctatttcgttcatcc
atagttgcctgactccccgtcgtgtagataactacgatacgggagggcttaccatctggccccagtgctgcaatgataccgcgagatcc
acgctcaccggctccagatttatcagcaataaaccagccagccggaagggccgagcgcagaagtggtcctgcaactttatccgcctcca
tccagtctattaatccacctggcggcgttgtgacaatttaccgaacaactccgcggccgggaagccgatctcggcttgaacgaattgtt
aggtggcggtacttgggtcgatatcaaagtgcatcacttcttcccgtatgcccaactttgtatagagagccactgcgggatcgtcaccg
taatctgcttgcacgtagatcacataagcaccaagcgcgttggcctcatgcttgaggagattgatgagcgcggtggcaatgccctgcct
ccggtgctctccggagactgcgagatcatagatatagatctcactacgcggctgctcaaacttgggcagaacgtaagccgcgagagcgc
caacaaccgcttcttggtcgaaggcagcaagcgcgatgaatgtcttactacggagcaagttcccgaggtaatcggagtccggctgatgt
tgggagtaggtggctacgtctccgaactcacgaccgaaaagatcaagagcagcccgcatggatttgacttggtcagggccgagcctaca
tgtgcgaatgatgcccatacttgagccacctaactttgttttagggcgactgccctgctgcgtaacatcgttgctgctgcgtaacatcg
ttgctgctccataacatcaaacatcgacccacggcgtaacgcgcttgctgcttggatgcccgaggcatagactgtacaaaaaaacagtc
ataacaagccatgaaaaccgccactgcgccgttaccaccgctgcgttcggtcaaggttctggaccagttgcgtgagcgcatacgctact
tgcattacagtttacgaaccgaacaggcttatgtcaactgggttcgtgccttcatccgtttccacggtgtgcgtccatgggcaaatatt
atacgcaaggcgacaaggtgctgatgccgctggcgattcaggttcatcatgccgtttgtgatggcttccatgtcggcaggaattcgaat
tcatacccaccggctccaactgcgcggcctgcggccttgccccatcaatttttttaattttctctggggaaaagcctccggcctgcggc
ctgcgcgcttcgcttgccggttggacaccaagtggaaggcgggtcaaggctcgcgcagcgaccgcgcagcggcttggccttgacgcgcc
tggaacgacccaagcctatgcgagtgggggcagtcgaagggcgaagcccgcccgcctgccccccgagcctcacggcggcgagtgcgggg
gttccaagggggcagcgccaccttgggcaaggccgaaggccgcgcagtcgatcaacaagccccggaggggccactttttgccggagggg
gagccgcgccgaaggcgtgggggaaccccgcaggggtgcccttctttgggcaccaaagaactagatatagggcgaaatgcgaaagactt
aaaaatcaacaacttaaaaaaggggggtacgcaacagctcattgcggcaccccccgcaatagctcattgcgtaggttaaagaaaatctg
taattgactgccacttttacgcaacgcataattgttgtcgcgctgccgaaaagttgcagctgattgcgcatggtgccgcaaccgtgcgg
cacccctaccgcatggagataagcatggccacgcagtccagagaaatcggcattcaagccaagaacaagcccggtcactgggtgcaaac
ggaacgcaaagcgcatgaggcgtgggccgggcttattgcgaggaaacccacggcggcaatgctgctgcatcacctcgtggcgcagatgg
gccaccagaacgccgtggtggtcagccagaagacactttccaagctcatcggacgttctttgcggacggtccaatacgcagtcaaggac
ttggtggccgagcgctggatctccgtcgtgaagctcaacggccccggcaccgtgtcggcctacgtggtcaatgaccgcgtggcgtgggg
ccagccccgcgaccagttgcgcctgtcggtgttcagtgccgccgtggtggttgatcacgacgaccaggacgaatcgctgttggggcatg
gcgacctgcgccgcatcccgaccctgtatccgggcgagcagcaactaccgaccggccccggcgaggagccgcccagccagcccggcatt
ccgggcatggaaccagacctgccagccttgaccgaaacggaggaatgggaacggcgcgggcagcagcgcctgccgatgcccgatgagcc
gtgttttctggatgatggcgagccgttggagccgccgacacgggtcacgctgccgcgccggtagcacttgggttgcgcagcaacccgta
agtgcgctgttccagactatcggctgtagccgcctcgccgccctataccttgtctgcctccccgcgttgcgtcgcggtgcatggagccg
ggccacctcgacctgaatggaagccggcggcacctcgctaacggattcaccgtttttatcaggctctgggaggcagaataaatgatcat
atcgtcaattattacctccacggggagagcctgagcaaactggcctcaggcatttgagaagcacacggtcacactgcttccggtagtca
ataaaccggtaaaccagcaatagacataagcggctatttaacgaccctgccctgaaccgacgaccgggtcgaatttgctttcgaatttc
tgccattcatccgcttattatcacttattcaggcgtagcaccaggcgtttaacgatcgtcttccttgctcgtcggtgatgtacttacag
ctcgaagtgcctcttcttgatggagcgcatggggacgtgcttggcaatcacgcgcaccccccggccgttttagcggctaaaaaagtcat
ggctctgccctcgggcggaccacgcccatcatgaccttgccaagctcgtcctgcttctcttcgatcttcgccagcagggcgaggatcgt
ggcatcaccgaaccgcgccgtgcgcgggtcgtcggtgagccagagtttcagcaggccgcccaggcggcccaggtcgccattgatgcggg
ccagctcgcggacgtgctcatagtccacgacgcccgtgattttgtagccctggccgacggccagcaggtaggccgacaggctcatgccg
gccgccgccgccttttcctcaatcgctcttcgttcgtctggaaggcagtacaccttgataggtgggctgcccttcctggttggcttggt
ttcatcagccatccgcttgccctcatctgttacgccggcggtagccggccagcctcgcagagcaggattcccgttgagcaccgccaggt
gcgaataagggacagtgaagaaggaacacccgctcgcgggtgggcctacttcacctatcctgcccggctgacgccgttggatacaccaa
ggaaagtctacacgaaccctttggcaaaatcctgtatatcgtgcgaaaaaggatggatataccgaaaaaatcgctataatgaccccgaa
gcagggttatgcagcggaagatatcgatgcataatgtgcctgtcaaatggacgaagcagggattctgcaaaccctatgctactccgtca
agccgtcaattgtctgattcgttaccaattatgacaacttgacggctacatcattcactttttcttcacaaccggcacggaactcgctc
gggctggccccggtgcattttttaaatacccgcgagaaatagagttgatcgtcaaaaccaacattgcgaccgacggtggcgataggcat
ccgggtggtgctcaaaagcagcttcgcctggctgatacgttggtcctcgcgccagcttaagacgctaatccctaactgctggcggaaaa
gatgtgacagacgcgacggcgacaagcaaacatgctgtgcgacgctggcgatatcaaaattgctgtctgccaggtgatcgctgatgtac
tgacaagcctcgcgtacccgattatccatcggtggatggagcgactcgttaatcgcttccatgcgccgcagtaacaattgctcaagcag
atttatcgccagcagctccgaatagcgcccttccccttgcccggcgttaatgatttgcccaaacaggtcgctgaaatgcggctggtgcg
cttcatccgggcgaaagaaccccgtattggcaaatattgacggccagttaagccattcatgccagtaggcgcgcggacgaaagtaaacc
cactggtgataccattcgcgagcctccggatgacgaccgtagtgatgaatctctcctggcgggaacagcaaaatatcacccggtcggca
aacaaattctcgtccctgatttttcaccaccccctgaccgcgaatggtgagattgagaatataacctttcattcccagcggtcggtcga
taaaaaaatcgagataaccgttggcctcaatcggcgttaaacccgccaccagatgggcattaaacgagtatcccggcagcaggggatca
ttttgcgcttcagccatacttttcatactcccgccattcagagaagaaaccaattgtccatattgcatcagacattgccgtcactgcgt
atttactggctcttctcgctaaccaaaccggtaaccccgcttattaaaagcattctgtaacaaagcgggaccaaagccatgacaaaaac
gcgtaacaaaagtgtctataatcacggcagaaaagtccacattgattatttgcacggcgtcacactttgctatgccatagcatttttat
ccataagattagcggatcctacctgacgctttttatcgcaactctctactgtttctccatacccgtttttttgggctagccctgtagaa
ataattgtttaactttaataaggagatataccatgggtaaaagcggaatttatcagattaaaaatactttaaacaataaagtatatgta
ggaagtgctaaagattttgaaaagagatggaagaggcattttaaagatttagaaaagggatgccattcttctataaaacttcagaggtc
ttttaacaaacatggtaatgtgtttgaatgttctattttggaagaaattccatatgagaaagatttgattattgaacgagaaaattttt
ggattaaagagcttaattctaaaattaatggatacaatattgctgatgcaacgtttggtgatacgtgttctacgcatccattaaaagaa
gaaattattaagaaacgttctgaaacttttaaagctaagatgcttaaacttggacctgatggtcggaaagctctttacagtaaacccgg
aagtaaaaacgggcgttggaatccagaaacccataagttttgtaagtgcggtgttcgcatacaaacttctgcttatacttgtagtaaat
gcagaaatggtggttctggtggtaccggaggtagcatggataaaaagtattctattggtttagacatcggcactaattccgttggatgg
gctgtcataaccgatgaatacaaagtaccttcaaagaaatttaaggtgttggggaacacagaccgtcattcgattaaaaagaatcttat
cggtgccctcctattcgatagtggcgaaacggcagaggcgactcgcctgaaacgaaccgctcggagaaggtatacacgtcgcaagaacc
gaatatgttacttacaagaaatttttagcaatgagatggccaaagttgacgattctttattcaccgtttggaagagtccttccttgtcg
aagaggacaagaaacatgaacggcaccccatctttggaaacatagtagatgaggtggcatatcatgaaaagtacccaacgatttatcac
ctcagaaaaaagctagttgactcaactgataaagcggacctgaggttaatctacttggctcttgcccatatgataaagttccgtgggca
ctttctcattgagggtgatctaaatccggacaactcggatgtcgacaaactgttcatccagttagtacaaacctataatcagttgtttg
aagagaaccctataaatgcaagtggcgtggatgcgaaggctattcttagcgcccgcctctctaaatcccgacggctagaaaacctgatc
gcacaattacccggagagaagaaaaatgggttgttcggtaaccttatagcgctctcactaggcctgacaccaaattttaagtcgaactt
cgacttagctgaagatgccaaattgcagcttagtaaggacacgtacgatgacgatctcgacaatctactggcacaaattggagatcagt
atgcggacttatttttggctgccaaaaaccttagcgatgcaatcctcctatctgacatactgagagttaatactgagattaccaaggcg
ccgttatccgcttcaatgatcaaaaggtacgatgaacatcaccaagacttgacacttctcaaggccctagtccgtcagcaactgcctga
gaaatataaggaaatattctttgatcagtcgaaaaacgggtacgcaggttatattgacggcggagcgagtcaagaggaattctacaagt
ttatcaaacccatattagagaagatggatgggacggaagagttgcttgtaaaactcaatcgcgaagatctactgcgaaagcagcggact
ttcgacaacggtagcattccacatcaaatccacttaggcgaattgcatgctatacttagaaggcaggaggatttttatccgttcctcaa
agacaatcgtgaaaagattgagaaaatcctaacctttcgcataccttactatgtgggacccctggcccgagggaactctcggttcgcat
ggatgacaagaaagtccgaagaaacgattactccctggaattttgaggaagttgtcgataaaggtgcgtcagctcaatcgttcatcgag
aggatgaccaactttgacaagaatttaccgaacgaaaaagtattgcctaagcacagtttactttacgagtatttcacagtgtacaatga
actcacgaaagttaagtatgtcactgagggcatgcgtaaacccgcctttctaagcggagaacagaagaaagcaatagtagatctgttat
tcaagaccaaccgcaaagtgacagttaagcaattgaaagaggactactttaagaaaattgaatgcttcgattctgtcgagatctccggg
gtagaagatcgatttaatgcgtcacttggtacgtatcatgacctcctaaagataattaaagataaggacttcctggataacgaagagaa
tgaagatatcttagaagatatagtgttgactcttaccctctttgaagatcgggaaatgattgaggaaagactaaaaacatacgctcacc
tgttcgacgataaggttatgaaacagttaaagaggcgtcgctatacgggctggggacgattgtcgcggaaacttatcaacgggataaga
gacaagcaaagtggtaaaactattctcgattttctaaagagcgacggcttcgccaataggaactttatgcagctgatccatgatgactc
tttaaccttcaaagaggatatacaaaaggcacaggtttccggacaaggggactcattgcacgaacatattgcgaatcttgctggttcgc
cagccatcaaaaagggcatactccagacagtcaaagtagtggatgagctagttaaggtcatgggacgtcacaaaccggaaaacattgta
atcgagatggcacgcgaaaatcaaacgactcagaaggggcaaaaaaacagtcgagagcggatgaagagaatagaagagggtattaaaga
actgggcagccagatcttaaaggagcatcctgtggaaaatacccaattgcagaacgagaaactttacctctattacctacaaaatggaa
gggacatgtatgttgatcaggaactggacataaaccgtttatctgattacgacgtcgatcacattgtaccccaatcctttttgaaggac
gattcaatcgacaataaagtgcttacacgctcggataagaaccgagggaaaagtgacaatgttccaagcgaggaagtcgtaaagaaaat
gaagaactattggcggcagctcctaaatgcgaaactgataacgcaaagaaagttcgataacttaactaaagctgagaggggtggcttgt
ctgaacttgacaaggccggatttattaaacgtcagctcgtggaaacccgccaaatcacaaagcatgttgcacagatactagattcccga
atgaatacgaaatacgacgagaacgataagctgattcgggaagtcaaagtaatcactttaaagtcaaaattggtgtcggacttcagaaa
ggattttcaattctataaagttagggagataaataactaccaccatgcgcacgacgcttatcttaatgccgtcgtagggaccgcactca
ttaagaaatacccgaagctagaaagtgagtttgtgtatggtgattacaaagtttatgacgtccgtaagatgatcgcgaaaagcgaacag
gagataggcaaggctacagccaaatacttcttttattctaacattatgaatttctttaagacggaaatcactctggcaaacggagagat
acgcaaacgacctttaattgaaaccaatggggagacaggtgaaatcgtatgggataagggccgggacttcgcgacggtgagaaaagttt
tgtccatgccccaagtcaacatagtaaagaaaactgaggtgcagaccggagggttttcaaaggaatcgattcttccaaaaaggaatagt
gataagctcatcgctcgtaaaaaggactgggacccgaaaaagtacggtggcttcgatagccctacagttgcctattctgtcctagtagt
ggcaaaagttgagaagggaaaatccaagaaactgaagtcagtcaaagaattattggggataacgattatggagcgctcgtcttttgaaa
agaaccccatcgacttccttgaggcgaaaggttacaaggaagtaaaaaaggatctcataattaaactaccaaagtatagtctgtttgag
ttagaaaatggccgaaaacggatgttggctagcgccggagagcttcaaaaggggaacgaactcgcactaccgtctaaatacgtgaattt
cctgtatttagcgtcccattacgagaagttgaaaggttcacctgaagataacgaacagaagcaactttttgttgagcagcacaaacatt
atctcgacgaaatcatagagcaaatttcggaattcagtaagagagtcatcctagctgatgccaatctggacaaagtattaagcgcatac
aacaagcacagggataaacccatacgtgagcaggcggaaaatattatccatttgtttactcttaccaacctcggcgctccagccgcatt
caagtattttgacacaacgatagatcgcaaacgatacacttctaccaaggaggtgctagacgcgacactgattcaccaatccatcacgg
gattatatgaaactcggatagatttgtcacagcttgggggtgacggatcccatcatcaccaccaccattgagcggccgcataatgctta
agtcgaacagaaagtaatcgtattgtacacggccgcataatcgaaattccctatcagtgatagagattgacatccctatcagtgataga
gatactgagcacgggagacccatgccatagcgttgttcggaatatgaatttttgaacagattcaccaacacctagtggtctcgttttag
agctagaaatagcaagttaaaataaggctagtccgttatcaacttgaaaaagtggcaccgagtcggtgctccgctgagcaataactagc
ataaccccttggggcctctaaacgggtcttgaggggttttttggcgagcatcacgtgctataaaaataattataatttaaattttttaa
tataaatatataaattaaaaatagaaagtaaaaaaagaaattaaagaaaaaatagtttttgttttccgaagatgtaaaagactctaggg
ggatcgccaacaaatactaccttttaccttgctcttcctgctctcaggtattaatgccgaattgtttcatcttgtctgtgtagaagacc
acacacgaaaatcctgtgattttacattttacttatcgttaatcgaatgtatatctatttaatctgcttttcttgtctaataaatatat
atgtaaagtacgctttttgttgaaattttttaaacctttgtttatttttttttcttcattccgtaactcttctaccttctttatttact
ttctaaaatccaaatacaaaacataaaaataaataaacacagagtaaattcccaaattattccatcattaaaagatacgaggcgcgtgt
aagttacaggcaagcgatcctagtacactctatatttttttatgcctcggtaatgattttcattttttttttccacctagcggatgact
ctttttttttcttagcgattggcattatcacataatgaattatacattatataaagtaatgtgatttcttcgaagaatatactaaaaaa
tgagcaggcaagataaacgaaggcaaagatgacagagcagaaagccctagtaaagcgtattacaaatgaaaccaagattcagattgcga
tctctttaaagggtggtcccctagcgatagagcactcgatcttcccagaaaaagaggcagaagcagtagcagaacaggccacacaatcg
caagtgattaacgtccacacaggtatagggtttctggaccatatgatacatgctctggccaagcattccggctggtcgctaatcgttga
gtgcattggtgacttacacatagacgaccatcacaccactgaagactgcgggattgctctcggtcaagcttttaaagaggccctagggg
ccgtgcgtggagtaaaaaggtttggatcaggatttgcgcctttggatgaggcactttccagagcggtggtagatctttcgaacaggccg
tacgcagttgtcgaacttggtttgcaaagggagaaagtaggagatctctcttgcgagatgatcccgcattttcttgaaagctttgcaga
ggctagcagaattaccctccacgttgattgtctgcgaggcaagaatgatcatcaccgtagtgagagtgcgttcaaggctcttgcggttg
ccataagagaagccacctcgcccaatggtaccaacgatgttccctccaccaaaggtgttcttatgtagttttacacaggagtctggact
tgactaggagtgcggttggaacgttggcccagccagatactcccgatcacgagcaggacgccgatgatttgaagcgcactcagcgtctg
atccaagaacaaccatcctagcaacacggcggtccccgggctgagaaagcccagtaaggaaacaactgtaggttcgagtcgcgagatcc
cccggaaccaaaggaagtaggttaaacccgctccgatcaggccgagccacgccaggccgagaacattggttcctgtaggcatcgggatt
ggcggatcaaacactaaagctactggaacgagcagaagtcctccggccgccagttgccaggcggtaaaggtgagcagaggcacgggagg
ttgccacttgcgggtcagcacggttccgaacgccatggaaaccgcccccgccaggcccgctgcgacgccgacaggatctagcgctgcgt
ttggtgtcaacaccaacagcgccacgcccgcagttccgcaaatagcccccaggaccgccatcaatcgtatcgggctacctagcagagcg
gcagagatgaacacgaccatcagcggctgcacagcgcctaccgtcgccgcgaccccgcccggcaggcggtagaccgaaataaacaacaa
gctccagaatagcgaaatattaagtgcgccgaggatgaagatgcgcatccaccagattcccgttggaatctgtcggacgatcatcacga
gcaataaacccgccggcaacgcccgcagcagcataccggcgacccctcggcctcgctgttcgggctccacgaaaacgccggacagatgc
gccttgtgagcgtccttggggccgtcctcctgtttgaagaccgacagcccaatgatctcgccgtcgatgtaggcgccgaatgccacggc
atctcgcaaccgttcagcgaacgcctccatgggctttttctcctcgtgctcgtaaacggacccgaacatctctggagctttcttcaggg
ccgacaatcggatctcgcggaaatcctgcacgtcggccgctccaagccgtcgaatctgagccttaatcacaattgtcaattttaatcct
ctgtttatcggcagttcgtagagcgcgccgtgcgtcccgagcgatactgagcgaagcaagtgcgtcgagcagtgcccgcttgttcctga
aatgccagtaaagcgctggctgctgaacccccagccggaactgaccccacaaggccctagcgtttgcaatgcaccaggtcatcattgac
ccaggcgtgttccaccaggccgctgcctcgcaactcttcgcaggcttcgccgacctgctcgcgccacttcttcacgcgggtggaatccg
atccgcacatgaggcggaaggtttccagcttgagcgggtacggctcccggtgcgagctgaaatagtcgaacatccgtcgggccgtcggc
gacagcttgcggtacttctcccatatgaatttcgtgtagtggtcgccagcaaacagcacgacgatttcctcgtcgatcaggacctggca
acgggacgttttcttgccacggtccaggacgcggaagcggtgcagcagcgacaccgattccaggtgcccaacgcggtcggacgtgaagc
ccatcgccgtcgcctgtaggcgcgacaggcattcctcggccttcgtgtaataccggccattgatcgaccagcccaggtcctggcaaagc
tcgtagaacgtgaaggtgatcggctcgccgataggggtgcgcttcgcgtactccaacacctgctgccacaccagttcgtcatcgtcggc
ccgcagctcgacgccggtgtaggtgatcttcacgtccttgttgacgtggaaaatgaccttgttttgcagcgcctcgcgcgggattttct
tgttgcgcgtggtgaacagggcagagcgggccgtgtcgtttggcatcgctcgcatcgtgtccggccacggcgcaatatcgaacaaggaa
agctgcatttccttgatctgctgcttcgtgtgtttcagcaacgcggcctgcttggcctcgctgacctgttttgccaggtcctcgccggc
ggtttttcgcttcttggtcgtcatagttcctcgcgtgtcgatggtcatcgacttcgccaaacctgccgcctcctgttcgagacgacgcg
aacgctccacggcggccgatggcgcgggcagggcagggggagccagttgcacgctgtcgcgctcgatcttggccgtagcttgctggacc
atcgagccgacggactggaaggtttcgcggggcgcacgcatgacggtgcggcttgcgatggtttcggcatcctcggcggaaaaccccgc
gtcgatcagttcttgcctgtatgccttccggtcaaacgtccgattcattcaccctccttgcgggattgccccgactcacgccggggcaa
tgtgcccttattcctgatttgacccgcctggtgccttggtgtccagataatccaccttatcggcaatgaagtcggtcccgtagaccgtc
tggccgtccttctcgtacttggtattccgaatcttgccctgcacgaataccagcgaccccttgcccaaatacttgccgtgggcctcggc
ctgagagccaaaacacttgatgcggaagaagtcggtgcgctcctgcttgtcgccggcatcgttgcgccactcttcattaaccgctatat
cgaaaattgcttgcggcttgttagaattgccatgacgtacctcggtgtcacgggtaagattaccgataaactggaactgattatggctc
atatcgaaagtctccttgagaaaggagactctagtttagctaaacattggttccgctgtcaagaactttagcggctaaaattttgcggg
ccgcgaccaaaggtgcgaggggcggcttccgctgtgtacaaccagatatttttcaccaacatccttcgtctgctcgatgagcggggcat
gacgaaacatgagctgtcggagagggcaggggtttcaatttcgtttttatcagacttaaccaacggtaaggccaacccctcgttgaagg
tgatggaggccattgccgacgccctggaaactcccctacctcttctcctggagtccaccgaccttgaccgcgaggcactcgcggagatt
gcgggtcatcctttcaagagcagcgtgccgcccggatacgaacgcatcagtgtggttttgccgtcacataaggcgtttatcgtaaagaa
atggggcgacgacacccgaaaaaagctgcgtggaaggctctgacgccaagggttagggcttgcacttccttctttagccgctaaaacgg
ccccttctctgcgggccgtcggctcgcgcatcatatcgacatcctcaacggaagccgtgccgcgaatggcatcgggcgggtgcgctttg
acagttgttttctatcagaacccctacgtcgtgcggttcgattagctgtttgtcttgcaggctaaacactttcggtatatcgtttgcct
gtgcgataatgttgctaatgatttgttgcgtaggggttactgaaaagtgagcgggaaagaagagtttcagaccatcaaggagcgggcca
agcgcaagctggaacgcgacatgggtgcggacctgttggccgcgctcaacgacccgaaaaccgttgaagtcatgctcaacgcggacggc
aaggtgtggcacgaacgccttggcgagccgatgcggtacatctgcgacatgcggcccagccagtcgcaggcgattatagaaacggtggc
cggattccacggcaaagaggtcacgcggcattcgcccatcctggaaggcgagttccccttggatggcagccgctttgccggccaattgc
cgccggtcgtggccgcgccaacctttgcgatccgcaagcgcgcggtcgccatcttcacgctggaacagtacgtcgaggcgggcatcatg
acccgcgagcaatacgaggtcattaaaagcgccgtcgcggcgcatcgaaacatcctcgtcattggcggtactggctcgggcaagaccac
gctcgtcaacgcgatcatcaatgaaatggtcgccttcaacccgtctgagcgcgtcgtcatcatcgaggacaccggcgaaatccagtgcg
ccgcagagaacgccgtccaataccacaccagcatcgacgtctcgatgacgctgctgctcaagacaacgctgcgtatgcgccccgaccgc
atcctggtcggtgaggtacgtggccccgaagcccttgatctgttgatggcctggaacaccgggcatgaaggaggtgccgccaccctgca
cgcaaacaaccccaaagcgggcctgagccggctcgccatgcttatcagcatgcacccggattcaccgaaacccattgagccgctgattg
gcgaggcggttcatgtggtcgtccatatcgccaggacccctagcggccgtcgagtgcaagaaattctcgaagttcttggttacgagaac
ggccagtacatcaccaaaaccctgtaaggagtatttccaatgacaacggctgttccgttccgtctgaccatgaatcgcggcattttgtt
ctaccttgccgtgttcttcgttctcgctctcgcgttatccgcgcatccggcgatggcctcggaaggcaccggcggcagcttgccatatg
agagctggctgacgaacctgcgcaactccgtaaccggcccggtggccttcgcgctgtccatcatcggcatcgtcgtcgccggcggcgtg
ctgatcttcggcggcgaactcaacgccttcttccgaaccctgatcttcctggttctggtgatggcgctgctggtcggcgcgcagaacgt
gatgagcaccttcttcggtcgtggtgccgaaatcgcggccctcggcaacggggcgctgcaccaggtgcaagtcgcggcggcggatgccg
tgcgtgcggtagcggctggacggctcgcctaatcatggctctgcgcacgatccccatccgtcgcgcaggcaaccgagaaaacctgttca
tgggtggtgatcgtgaactggtgatgttctcgggcctgatggcgtttgcgctgattttcagcgcccaagagctgcgggccaccgtggtc
ggtctgatcctgtggttcggggcgctctatgcgttccgaatcatggcgaaggccgatccgaagatgcggttcgtgtacctgcgtcaccg
ccggtacaagccgtattacccggcccgctcgaccccgttccgcgagaacaccaatagccaagggaagcaataccgatgatccaagcaat
tgcgattgcaatcgcgggcctcggcgcgcttctgttgttcatcctctttgcccgcatccgcgcggtcgatgccgaactgaaactgaaaa
agcatcgttccaaggacgccggcctggccgatctgctcaactacgccgctgtcgtcgatgacggcgtaatcgtgggcaagaacggcagc
tttatggctgcctggctgtacaagggcgatgacaacgcaagcagcaccgaccagcagcgcgaagtagtgtccgcccgcatcaaccaggc
cctcgcgggcctgggaagtgggtggatgatccatgtggacgccgtgcggcgtcctgctccgaactacgcggagcggggcctgtcggcgt
tccctgaccgtctgacggcagcgattgaagaagagcgccggcggcatttcgagagcctgggaacgatgtacgagggctatttcgtcctc
accttgacctggttcccgccgctgctcgcccagcgcaagttcgtcgagctgatgtttgacgacgacgcgaccgcaccggatcgcaaggc
gcgcacgcggggcctcatcgaccaattcaagcgtgacgtgcgcagcatcgagtcgcgcctgtcgtcggccgtgtcgctcactcgcttga
aggggcacaagatcgtcaacgaggacggcacgaccgtcacgcatgacgacttcctgcgctggctgcaattctgcgtgacgggcctgcac
catccggtgcagctccccagcaacccgatgtacctggacgccctggtcggcggacaggaaatgtggggcggggtagtgcccaaggtcgg
ccgcaagttcgtccaggtggtcgctctcgaaggcttccccttggagtcctatcccggcatcctgacggcgctcggcgagctgccctgcg
agtatcggtggtcgagccggttcatcttcatggaccagcacgaagccgtgaagcacctcgacaagttccgcaagaagtggcggcagaag
attcgcggcttcttcgaccaggtgttcaacacgaacaccggcccggtcgatcaggacgcgctttcgatggtggccgatgctgaggcggc
cattgccgaagtcaacagcggcatcgtggccgtgggctactacaccagcgtcgtcgtgctgatggatgaggaccgcacgcgcctggaag
ctgcggcccgcgatgttgaaaaggccgtcaaccggttgggctttgccgcgcgcatcgagtccatcaacaccctggacgccttccttggt
agtttgccgggccacggcgtggaaaacgtccgccggccgctcatcaacacgatgaacctggccgacctgctgccgaccagcaccatctg
gaccggcaacgcgaacgcgccatgcccgatgtacccgccgctgtcgccggcgctcatgcactgcgtcacgcaaggatcaacgccgttcc
ggctgaacctgcacgtgcgcgacctcggccacacctttatgttcgggccgaccggcgcaggtaaatcgacgcacctggcgatcctcgcc
gcgcagctccgtcgctatgccggcatgtcgatcttcgcctttgacaagggcatgtcgatgtacccgctggccgccggcatccgtgcggc
cacgaagggcaccagcggcctgcacttcaccgtggcggccgacgacgaacgcctggcgttctgcccgttgcagttcctgagcaccaagg
gcgaccgtgcttgggcgatggagtggatcgacaccatcctggcgttgaacggcgtcgaaacgaccccggcccagcgcaacgaaatcggc
aacgcgatcatgagcatgcacgccagcggcgcgcgcacgctctccgagttcagcgtgacgattcaggatgaggcgatccgcgaggcgat
ccgccagtacaccgtcgatggcgcaatgggccatctgctcgacgccgaagaggacggcttggcgctgtccgactttacagtgttcgaga
tcgaagagctgatgaacctcggcgagaaattcgccctgcctgtgttgctctacctgttccgccgtatcgagcgcgccctgacgggccag
ccggccgtcatcatcctggacgaagcctggttgatgctcggccacccggcattccgcgcgaagatcagggaatggctcaaggtgctgcg
taaggccaactgccttgtgctgatggcaacgcagagcctgtccgacgccgccaacagcggcatcctggacgtgatcgtggaatcgaccg
cgaccaagattttcctgccgaatatttacgccagggatgaggacacggcggccctgtaccgccgcatgggcctgaacgctcgccagatc
gagattctggcccaggccgttcccaagcgtcagtactactacgtgtcggaaaacggccgccgtctctacgacctggcacttggcccgct
cgcgctcgcgttcgtcggcgcatccgacaaggaatccgtcgccatcatcaagaacctggaagccaagttcggcgaccagtgggtggatg
aatggctgcgtggccggggcctcgcccttgatgaatacctggaggcagcatgagttttgcagacacgatcaagggcttgatcttcaaga
agaagcccgcaacggccgcagcagcggcgacgccggccgcgaccggcccgcaaaccgacaacccgtacctgacggcgcggcgcacctgg
aacgaccacgttggttccgttgtgtcgcaaaagcagacctggcaggttgtcggcatcctttcgctgatgatcgtcctcgcggcggtcgg
cggcatcatccacatcggcagccagtcgaagttcgtgccctatgtctacgaggtagacaagctcgggcagacggccgccgtggggccga
tgaccagggcgtcgaaagccgatccgcgtgtcattcacgcctcggtggctgagttcgtcggcgatgctcgcctggtgacgccggacgta
gctttgcagcgcaaggccgtctaccgcctctatgccaagctcgggccgaatgacccggccaccgccaagatgaacgaatggctcaacgg
caccgccgacgccagcccgttcgctcgcgcggccgtcgaaacggtcagcaccgaaatcacttccgtaatcccgcagacgcccgacacct
ggcaggtcgattgggtcgagacgacgcgcgacaggcaaggcgtggtgaaaggccagcccgtgcgcatgcgggccttggtgacggtctac
gtcgtcgagccgacggcggacaccaaggaagaacaactgcgaaacaacccggccgggatctacgtccgggacttctcctggtcgagact
tctgtgaggcactgaattatgaaaaaggaactgtttgctttggtcctggccgcgtccgttagcgtgcctgcatttgccgccgatcccgg
cgcggacctgactgacctctatttttccggcaagaacccggagctgaccgcgcaagagcgggcggccatcgccatcgccaagaagtggg
aggcgggtaccgccggcatgcggccggtggccggccccggtggttcggtgcgcttcctgttcggcgcgcagcagccgagcatcgtatgc
gccgtgctgcaagtgtgcgacgtggccctgcaacccggcgagcaagtcaactcgatcaacctgggcgacaccgcccgttggacggtcga
gccggccattaccggcagcggcgcgaacgaaacccagcacctcatcatcaagccgatggatgtgggcctggaaaccagcctggtcgtga
ccacggaccgccgcagctaccacatgcgcctgcgctcgcatcgcacgcagtacatgccgcaggtgtcgttcacctacccggaagatgcc
cttgcgaagtgggacgccatcaagaaccgcgaacagcgggatcgcgtcgagaaaaccattccgcagaccggcgagtacctgggcaacct
gagcttcaactactccgtcagcgggtccacgtcgtggaagccggtgcgcgtctacaacgacggcaagaaaaccatcatccagatgccgc
actcgatggaacagaccgaagcgccgacgctcctggtcgttcgcagggagggcggcctgttctccgacgatgaaacggtgatggtcaac
taccgggtccagggcgaccgctacatcgtcgatacgattttcgacaaggccatcctcatcgcgggcgtgggcagcagccaggaccgcgt
gaccatttcaagggggaactaaaccatgcgtaagattctgaccgtcatcgcactcgcggccacgttggccggctgcgcgacctccaagt
acggcagcttcgtccaggacgcgccggccgcctacaaccagaccattgcgaccgacgcggtgaagcagctcgtcaagctctacccgccg
gcgcaaaccaagctggaattgcagcaggctacgcccgatccgttcggcattgccctggtcactgaccttcgcgcccagggctatgctgt
catggagtacaagcccgacggcaacgcggccgcagctccggctgctgcgtcctcggccgctgcgaagccggcaacgccgcaagcccagg
gcggctatccgctgcgctacgtgctggaccaattcagcgacagcaacctgtatcgcctgaccgtcatggtcggctctcaatcgctcacg
cgcgcctacctcgcccaaaacaacacgatggtcccggccggcgcatgggttcggaaggagtaagccaatgagcgaagatcaaatggcac
cggacgcatcgccagatgcggtcaagccgaaaagcggggttcgccgcgtcaacaacatgccgatgtacctcatcggcggtgtgctcggc
atcttcctgctggtgatggccctggttgctgcggatcgcgctgcgcagcagaaccagccgggagctgcgaaggctgagaaggccggcag
caccagcatgtttgccgacgaaattgccggcaaacagcaggacggcatcatcaaggccaagccgctggagattccgccggaacaaaccg
cccagcaaccgacgacggagctgacgccagccccggcgcagggaacgactatcacggtcgcacggcccgagaacctggaccagcccccg
acgccgccgcagggtgcgcgcaacgaggacctggaccgcatccgcatggcgaagttgcagatgctggaagaggcgatcaaggccaagac
gacggtgcgcatcgacgcgccgcgcagccagggcagcgccggcggcggtgctccgcagggccgcgaggaaacccttgcgcgcatccagg
agctgcgtcggcaggctgagaacgcccgcgccaccgatccgaccgccgcctatcaggccgcgcttgcgcaggctcgcacgatgggcggc
gcggcagggggtggcggtatgggcggctcgggtgcgccgaccctcgtgcagacctcgaaccgcagtggtggcggcgctggctatgggtc
gttcgacaaccgcagcgagggcgaccgttggcggctcgactcccagccggaagcacctgcaacgccctatgtgctgcgcgctggcttcg
tcgttccggctacgcttatctcgggcatcaactccgatctgccaggccaaatcatggcccaggtatcgcagtcggtgtacgacacggcg
accggcaagcacatgctcatcccccaaggctcgcgcctggtgggcagctactcgaacgatgtggcctacgggcagaagcgcgttctggt
ggcatggcagcgcatcatcttccccgacggcaaggcaatggacattggggccatgccgggcggcgatagcgctgggtatgcaggcttca
acgacaaggtcaacaaccactacttccgcaccttcgcatcggcattcctcatgtcgggcgtcgttgcgggcatcagcttgagtcaggac
cgtggcaacagcaacagcggttacggacgacaagacgcgggttccgcgatgagtgaagcgttgggtcaacagctcggccaagtaacggc
gcagatgatcgccaaaaacttgaatatcgcgccgacgctggaaatccgtccgggctatcgcttcaacgtcattgtcacgaaagacatga
cgttttctaagccctaccaggcgtttgactattaactccaaggagtaacttatgaagaagctcgctaagaatgttttagccgctaaagt
agctctggtgctggccctctcggtcggcaccttggcggtcacgcctgcgcaagcgggcattccggtcatcgacggcaccaacctgtcac
aaaccactgtcaccgcgattcagcaggttgcgcaggtccagaagcaaatcgaggaataccggacgcagttgcagcagtacgaaaacatg
ctgcaaaacacggtggccccggccgcctacgtgtgggaccaggcgcagtccaccatcaacggcctgatgagcgccgttgataccctgaa
ctactacaagaaccaggcgggcagcatcgacgcttacctgggcaagttcaaggacgtgtcctactacaaggggtcgccgtgcttctccc
tgtcgggctgctcggaaagcgagcgcaaggcgatggaagagaaccgccgcctggcgtccgaatcgcagaaaaaggccaacgatgcgctg
ttccgtggcctcgatcagcagcagagcaacctcaagtccgacgccgccacgctggagcaattgaagggcaaggcgacgacggcgcaggg
ccagttggaagccctcggctacgccaaccagttcgccagccagcaggccaaccagctcatgcaaatccgtggccttctgcttgcgcagc
agaacgccatcgccacgcagatgcaggcccagcaggaccggcaggcccagcaggacgctgcgggcgcgaagctgcgcgagggttcgtac
cgcgcaagcccgtctaagacctggtgaggggaggcgcgatgaagaaatccaacttcatcgcagttgccgcgctggccgccgtcatggcg
gccagcctggcaggctgcgacaacaagcccgacaccgacaagctgacctgcgccgatctgccgaaggtcacggatgccgctcaacgcgc
ggagctgttgaagaagtgcccgcgcggagaaccgggaggcttcaagcccagcgaaaagaaagagtggtgatgacgtatgaaaatccaga
ctagagctgccgcgctcgcggtcctgatgctggccttgatgccggtagcggcatacgcccaaatcgacaattcgggcatcctcgacaac
gtattgcagcgctaccagaacgccgcgagcggctgggccactgtcgtccagaacgccgcaacctggctgttctggaccttgaccgtgat
tagcatggtctggaccttcggcatgatggcactgcgcaaggccgacattggcgagttcttcgccgagttcgtgcggttcaccatcttca
ccggcttcttctggtggctgctgaccaacggcccgaatttcgcgtcgtccatctatgcgtccctgcggcagattgcaggccaggcaacg
gggttggggcaggggctttcgccgtccggcatcgtcgatgttggcttcgagattttcttcaaggtgatggacgaaacctcgtactggtc
gccggtcgatagcttcgtcggtgcctcgttggcggccgccatcctctgcatcctggccctggtcggcgtgaatatgcttctgctcctgg
cgtccggatggattcttgcctacggcggtgtgttcttcctgggcttcggcggctcgcgctggacctcggacatggcgatcaactactac
aagaccgtcctcggggtcgccgcgcagctcttcgcaatggtgctgctcgtaggcatcggcaagaccttcctcgatgactactacagccg
catgagcgaaggcatcaacttcaaggaacttggagtgatgctgatcgtcggcctgatcctgctcgttctggtcaacaaggtgccgcagc
tcatcgccggcatcatcaccggcgcgagcgtcggcggtgctggtatcggccagttcggcgctggcacgctcgtcggtgcggccgcgacg
gccggcgcggcaatcgcaactggcggcgcatctatcgcggccggcgctgcggcggcggccggtggcgcgcaggccatcatggcggccgc
gtcgaaggccagcgataacgtctctgccggcactgacattctgtcgagcatgatgggcggcggcggtggcggcggcggtggtagcgccg
gcaccagcggcggcgacggcggcggctcgggtggcggcggtggctcgggcggcggtgaaaccccgatggcctcggccgccggcgacaac
agcagcggcgcacgcggcggcagttcgggcggcggctcgggtggtggccgttcgtctggcggtatcggtgccacggcggccaagggcgg
ccggatcgcggccgataccgtcgccaacctggcgaaaggtgccggctcgattgccaaggccaaggccggcgaaatgcgcgcatcggccc
aggaacgcatcggcgataccgtaggcggcaagatcgcgcaggcaattcgcggcgcgggtgcggcggcgcagaccgctgcaaccgtcgcc
gatagcaacagccaggcgcaggaacaacctgcaccggcacccgcaccgtcgttcgacgacaacagcctttccgcaagcaacaacaggga
agcggccgccgacgcggattccgaagtggcgagcttcgtcaacaagcccgcccaatcctgaaacgactcttaggagctacgaccatgca
actgaaaaaagcgttctcgtcggccgccctggtggtggccttgggcctcggcgcaactggctcggccagcgcgcaagacgtgctgacgg
gcgatacccgcctggcctgcgaggccattctgtgcctgtccacgggcagccggcccagcgagtgcagcccgtcgctctcgcggtacttc
ggcatccacaagcgcaagctgtcggacacgctcaaggcgcggctgaacttcctcaacctctgcccggtatcgaaccagacgccggaaat
gcagacgctcgtttcctcgatttcgcgcggggccgggcgctgcgatgcgtcctcgctgaactccgtgctgcgtgagtggcggagctggg
acgaccagttctacatcggcaaccgcctgccggactactgcgcggcctacaccggccatgcctataccgacttcaacacgaccgcgccg
cgctacgtcggcacgccggaagagggcggctattggatcgaggcggccgactacgaccgcgcgctcaaggagtacgaggcgaagctgaa
agagcggcagcagcagtacggtcgctatggcagcgacgcctaccgtcggttcgagcggtaaggggaggggatagcgatgccgtttgcca
agctgctggcacggaacgctctgccggtggtcgccctggtggcggccactggcttcggtgcggcggatgcgaccgccgcacggctcttc
cccgatctgtcggaacagatggaagagcgcgttgtgtgctcggtgtctgcggccgcgaagtacgagattccggccaacattcttctcgc
cattcgggaaaaggagggcggcaagccgggccagtgggtcaagaacaccaatggcacctatgacgtgggcgagctgcaattcaacaccg
cctacctgggcgacctggcgaagtatgggatcacggcccaggacgttgctgcggcaggctgctatccctatgacctggcggcctggcgg
ttgcgcgggcacattcgcaacgacaggggcgatctgtggacacgcgccgctaactatcactcgcgcacgccgtcgaagaacgcgatcta
tcgcgccgatctgatggtgaaggccgacaagtgggcgaagtggctggatgcgcgtttcgtcaccgtcaactatggccccagctcgccgg
cgcagccggcagggaaggggaccacacttgcggccgctgatacgtcggcagcagcgccggccgaagcgcagccgatgaagcaaggccgg
atcacccgcaccagcctccgcagctcgggttacgtaccccggcagctcatcatcaacaacacgccataaggaggaacggccgtttagcg
gctaaagcctatgggcattcgcaacctgacgcagcgatacatgaacggggccagggcctacgcggcctgggcggcatcgcaggcgaaag
cgccgtttgatcttctggtactgggcatcgggcctgtcatcgtctttggcctggtcgcgcatacgctgctcgcgttcctgcccacatgg
gccatgtacgccgccggcgctctgctggtcctcgcggccctgcctttggcgctgcacgtcctccgggaatacgcgctgcgctatgggcg
caaatagcgccctgcagggcgttcttactccaagggggagggcatgaatacacgcgccatgaacgacgccagcggccgggcctcgctgc
ctgccatggtgatcgccgacggcaccattgaagccttgaagtggctcgccttgcttgccatgaccggggatcacgtcaacaagtacctg
ttcaacggtacgctgccatatctgttcgaggcggggcgcttggccctgcctcttttcgttttcgtcctggcgtacaacctcgcccgccc
gggcgcgctcgagcgcggtttgtacgggcgagcgatgaaacgcctgttggccttcggcctggtcgcctcggtcccgttcattgcgttgg
gtggagtggtgggcggatggtggccgctgaacgtcatgttcacgctgttggccgcaaccgcgatgctctacctggtcgagcgcggccgc
tcggtcgctcctatagcgctgttcgtcgtggccggcggcctggtcgagttctgttggccggcgctgctgctggccgcgtctgtctggtt
gtacctcaagcgcccgacgtgggcggccgcgttgatggcgctgctgtcttgcgcgtccctgtggtacatcaatggcaacctttgggcgc
ttgctgttgtgcccctggtgatcgtcgccgccggcgtcgatcttcgtgtcccgcgcctgcgctgggccttttacacgtactacccgctg
catcttgccgctctttggctgatccgcattccgatgcgcgaggcgggctacttgtttttcacctgacctttgagattccaatatgcaat
tgctcaagaaatgcaccatcgcggccctgccgctgctcgccctgtccggctgcgcactgctgaacatccccatgccgacgccgcccggt
tcgaccccgccggaaatgctgaccgtgccagtggcgcaaatctgccgcgacgctgacaagaaccctgttcgggcaacggagctgtacgg
caagaaagggttgtcggccaccggcaaggtgcaggtgatttccgaaggcttcaagcctcgctatcgggtgctgctgcgcgctggcagcg
cctcggtccatgctgggaccgataaccagctcgccatcaagtcggtttccaccggccagaccacgcgcgtcactggcaccgtgaaggac
gtgtcctacgaccataacggctgctcgatctcgcttgacgatgcgaagttctactgaggggagggcggcggatgctgacacggttgaag
ggcttccttgctcgtcgccgcgagttgaaggaactggatgtgtccgtggtgagccggccccggccggctccggcggaattggtccaggt
tgatgcacgcgaggccgtttggcgcgtgccggtgcccggccaggccgaccgcttcatgtcggccaagcctggcgcgatcaacgatgaaa
tgttcgtggttcgggtggacaccgaagcgttctatcgggcttggctgcgcagcagctcgacgggccgcgaaacgcggtcggacaactgc
ccgctgcgctcggaaatgccgcaggactacaagttcaagcacgccgtccagggcttcgcgcacggcagggaaaatcctgtgccgctggc
cttcgccggcgcgcaccaggagcgccaccgggtggacattggtttcagcaacggggtcacgcgctcgttctggctgattgccaacaagg
ctccgtcgttcccgatccaggtccacggccgggagtcggccgagctgctgaacaaggtttgcggcctcgatcctgcgccgctgtcgttc
acggaactgttcgcgcaggcccaacgccaggctccgcaggtcgccacaccggcccggcctgcgccggcagcggccacccggccagctcc
caaggtgcagccacgccccggccgaagcggcccgcgcaaaggccgcggactctgactacaaccgtgcgcaaggcgcattagggaggatg
tatgtatgtaatcgcctgcggcatcgttgccggcttggcggctgcggtggccctgttgggcttcacgccgatgatggaggcgcttgccg
ccggcgaacgccgcaaggcactcgcgcaatggacgcggacgatgttcctggtgctgctgcctgtcgtgctgatgtgcgcgcccatcggg
tccagcatttacgacgccgtgcaagcggacgctggcaagcccatcgctttccacaacggccggatcacggtcgtcatggccctggtcgg
cagcttggccgttgtcctggtcgcggctgcgcgtgcggtggtcaaccgcaagcatgccagcttctggttcgtcggctgggtgatggcgt
cggttttggccggcggcgtcggcgcgatcgccagcgcgaagcaactggcgttcctcggcgaacatagcggcatggtggccttcggcttc
ttccgcgaccaggtgaaggacatgcactgcgatgcggacgtgatcctggcccggtgggatgaaaaggcgaactcgccggtggtctaccg
ctgcccgaaggcgtacctgctcaacaggttcgcatccgcgcccttcgtgccctggccggactacaccgagggggaaagcgaggatctag
gtagggcgctcgcagcggccctgcgggacgcgaaaaggtgagaaaagccgggcactgcccggctttatttttgctgctgcgcgttccag
gccgcccacactcgtttgacctggctcgggctgcatccgaccagcttggccgtcttggcaatgctcgatccgccggagcgaagcgtgat
gatgcggtcgtgcatgccggcgtcacgtttgcggccggtgtagcggccggcggccttcgccaactggacaccctgacgttgacgctcgc
gccgatcctcgtagtcgtcgcgggccatctgcaaggcgagcttcaaaagcatgtcctggacggattccagaacgattttcgccactccg
ttcgcctcggcggccagctccgacaggtccaccacgccaggcacggccagcttggcccctttggcccggatcgacgcaaccaggcgctc
ggcctcggccaacggcaagcggctgatgcggtcgatcttctccgcaacgacgacttcaccaggttgcaggtccgcgatcatgcgcagca
gctcgggccggtcggcgcgtgcgccggacgccttctcgcggtagatgccggcgacgtagtacccggcggcccgcgtggccgctacaagg
ctctcctggcgttcaagattctgctcgtccgtactggcgcgcaggtagatgcgggcgaccttcaaccttcgtccctccggttgttgctc
tcgcgtcgccatttccacggctcgacggcgtgcggatcggaccagaggccgacgcgcttgcctcgcgcctcctgttcgagccgcagcat
ttcagggtcggccgcgcggccgtggaagcgataggcccacgccatgccctggtgaaccatcgcggcgttgacgttgcgcggctgcggcg
gccggctggccagctccatgttgacccacacggtgcccagcgtgcggccgtaacggtcggtgtccttctcgtcgaccaggacgtgccgg
cggaacaccatgccggccagcgcctggcgcgcacgttcgccgaaggcttgccgcttttccggcgcgtcaatgtccaccaggcgcacgcg
caccggctgcttgtctaccagcacgtcgatggtgtcgccgtcgatgatgcgcacgacctcgccgcgcagctcggcccatgccggcgagg
caacgaccaggacggccagcgcggcagcggcgcgcagcatggcgtagcttcggcgcttcatgcgtggccccattgctgatgatcggggt
acgccaggtgcagcactgcatcgaaattggccttgcagtagccgtccagcgccacccgcgagccgaacgccggcgaaaggtactcgacc
aggccgggccggtcgcggacctcgcgccccaggacgtggatgcgccggccgcgtgtgccgtcgggtccaggcacgaaggccagcgcctc
gatgttgaagtcgatggatagaagttgtcggtagtgcttggccgccctcatcgcgtcccccttggtcaaattgggtatacccatttggg
cctagtctagccggcatggcgcattacagcaatacgcaatttaaatgcgcctagcgcattttcccgaccttaatgcgcctcgcgctgta
gcctcacgcccacatatgtgctaatgtggttacgtgtattttatggaggttatccaatgagccgcctgacaatcgacatgacggaccag
cagcaccagagcctgaaagccctggccgccttgcagggcaagaccattaagcaatacgccctcgaacgtctgttccccggtgacgctga
tgccgatcaggcatggcaggaactgaaaaccatgctggggaaccgcatcaacgatgggcttgccggcaaggtgtccaccaagagcgtcg
gcgaaattcttgatgaagaactcagcggggatcgcgcttgacggcctacatcctcacggctgaggccgaagccgatctacgcggcatca
tccgctacacgcgccgggagtggggcgcggcgcaggtgcgccgctatatcgctaagctggaacagggcatagccaggcttgccgccggc
gaaggcccgtttaaggacatgagcgaactctttcccgcgctgcggatggcccgctgcgaacaccactacgttttttgcctgccgcgtgc
gggcgaacccgcgttggtcgtggcgatcctgcatgagcgcatggacctcatgacgcgacttgccgacaggctcaagggctgatttcagc
cgctaaaaatcgcgccactcacaacgtcctgatggcgtacttacccaaagaacagctaggagaatcatttatgctcagcacacttccac
aagctcatgcaactttcttgaaccgcatccgcgatgcggtcgcttccgatgttcgcttccgcgctcttctgatcggcggctcttacgtt
cacggaggactcgatgagcactccgatttggatttcgacatcgttgttgaggacaactgctacgcagatgtcttgtctacacgcaagga
ttttgccgaggcactgcccggcttcctcaacgcgttcaccggcgaacatgtaggagaaccgcgccttctgatctgcctatatggtccgc
cactgctacacatcgatttgaagttttctcttgcttccgatctcgaccagcaaatcgagcggcgggcggttctgtttgctcgtgatccg
gcagagatcgagaagcgcattgaggcggcagcggtggcatggccaaaccgtccctccgagtggttcgaagcacgttgtcagcgccagtg
atataagacggtaattcaccatttggattgtccgctccacccaacatgttgtttccttaaggttctcacaccagaaaggacatcaacat
gctgagcagagaggacttttacatgataaagcaaatgcgccagcagggcgcgtacattgtcgatattgcgactcagattggttgctctg
aacggacggtcagacgctacctcaaataccctgaaccgccagccagaaagacccgccacaaaatggttaagctgaaaccgtttatggat
tacatcgacatgcgcctggcagagaatgtctggaatagtgaggttatctttgcggagattaaggcaatgggttatacgggcggacgttc
catgctgcgttactacatccagcccaaacgtaaaatgcgtccgtcaaaaagaacagttcgcttcgaaactcagcctggataccagctcc
agcacgactggggcgaagttgaggtggaggttgccgggcaacggtgcaaagttaactttgcggttaatacgctggggttctcccgccgc
ttccatgtcttcgccgcaccaaaacaggatgctgagcatacctacgaatcactggttcgcgccttccgctacttcggtggttgtgtgaa
aacggtgctggttgataaccagaaggctgcggtgctgaagaataacaacgggaaagtcgtgttcaactccggattcctgttgctggccg
accactataacttcctgccacgggcatgccgtccacgcagggccagaacaaaaggtaaggttgagcggatggtgaaatacctcaaggag
aacttcttcgttcggtaccgcaggttcgacagcttcactcatgttaatcaacaactggagcaatggatagccgatgtggctgacaaacg
ggaacttcgccagttcaaagaaacgccggaacagcgcttcgcgctggagcaggaacatctgcagccgttaccggatacggacttcgata
ccagttacttcgacatccgccatgtgtcctgggacagctatatcgaggttggtggtaatcgttacagcgttcccgaagcgctgtgtggt
cagccggtatcgatacgaatatcgctggatgacgagttgcggatctacagtaatgagaaactggtggcctcacatcgcctctgttcggc
atcgtctggctggcagacagtgccggagcatcacgccccgctctggcagcaggtcagtcaggtggaacatcgaccactgagtgcctatg
aggagctgttgtgatgcatgagctggaagtcctgctgagtcgcctgaaaatggagcatctgagttatcacgttgaaagcctgctggaac
aggcagctaaaaaagagctgaactaccgggagttcctgtgcatggcgctacagcaggaatggaacggcaggcatcagcgcggtatggag
tccaggctgaagcaggctcgtctgccgtgggtcaaaacgctggagcagttcgactttaccttccagccgggcatcgaccgtaaggttgt
ccgggaactggctggtctggcgttcgtggagcgcagcgaaaacgtgatcctgctgggacctcctggtgtcggaaaaactcatctggcca
tagctcttggcgtgaaagcggtggatgcgggacatcgggtactgtttatgccactggacagactgatcgcgacactgatgaaagcgaaa
caggaaaaccggctggagcgtcagctgcagcaactgagttatgcccgggtgttgatcctggatgaaataggctatctgccgatgaacag
agaggaagccagcctgttcttccggctactgaaccgtcgatatgaaaaagcgagcatcatactgacgtcaaacaaagggttcgcagact
ggggagaaatgttcggagatcacgtgctggcaacagcgatactggatcggttgctacatcactcaaccacgctgaatatcaaaggagag
agttaccggttaaaagagaaacgtaaagctggagtgctgaccaaaaacacaacgccaatcagtgatgatgaaatggtgaaaagcggaca
gcatcagtaacgaaagtatcttagcgggcatgaaaatggcaaataacggtcaaacatcgtggcgttgacaacgtgcctggatctggcta
cactatgcggccaccaagctcgcccgtggcgagctttacgaagcgatcggcatgctcggtttcttccgtgagcaagtgttaggaccttt
gctctaccgtcgcgctggaaaggaccagcgcggagtgaggcgattggaaacccttcgactggatgaagagcgcagactagccaccacca
ttgcgctgcacgatgcgttgtctgtcagggatgccatcaaagcatctgcctccatctatctcgacctccgagccgccgatccgtcgttg
gaaccgacaacgcatatgccaggtcttctgtacgacttaatagaacgtgcggtaccaggcacgcctaaccgtcagtgagattggatgag
tgaacgatattgatcgagaagagccctgcgcagccgctgccgtgcccgagagcatggcggctcacgtgatgggatacaaatgggcgcgt
gataaggttggtcagtccggctgcgcggtctatcggctgcatagcaagaattctgccgtgttatggaactgtcttgaggagttcgaacc
ttcgcttcaggagaggcttgttgcgcaatatggcattgccgatccggataggcgcaagctgcaatttcatctcctgctggacgaacttt
tctaaggcgatgccccctcgacctcgatcagggaggcgttcaggacgactcacaaagaaagccgggcaatgcccggctttttctgctgc
tacctccgtagtcgtaaggtcgttgcaggtgctcgggtgcggtacaactcgccggtcgccagctcaagcgcgatcacgtcgttgccgtc
gtagttgacgatgatgctgttgggccgactgtcctcacgcttcgcagggagaggccagccttcaatcgaagccggcgcaagctcgtagt
gcttcccggtttcgacgctgcgcagcgtccaggtcctgcaaccggccacgccggtcgcagaaaccacggcgagcgagccgcgaaaatcg
tgcgggtacgcctcgatgttcatacgcctcctagatcgagcgcgagcgtttctgctcggccttggccgcctgttcctgggacacctcgc
cgatgaccttgccctggccccggctgtaggcgatttcgtagttcttgccgacaaccggcggcttctcaaagatgccccggctgtgtttc
acgatcccgccttcgctgaactggtagacgttgcgcccatcgtcgtgcagcacctggccgacgtgcttgtgcgggtggacgtttttgct
tgcgtccttcgcatcgctcaactggtgaatgcctttcggtagccccgcctcgggcaacaccttcatggtcagccattcgccgttcacta
cctggtccacctggcggctgccgttcatgacggcgatcttgacgctgccctcgggtttcatgatgacgccagggcttgccgatgtgcgt
ggtgccccgatctgtactttgttcatacgctctagttctccttagtaggttctcgcgcggcgttgccgctgttcttgctgctcgatgtc
ttgctgcttgagctgctgcaccttctgccgctggccctcgtcgagaagcaccttgccgacagcacttctcacctggcgttcaaccccgt
ccttgcccaggctgctgcgctcggacaggtcgttgaaatcggtgtgcttcttcatgttcgacagggcggcgagctggccatcgttcaac
agcgattccttgagcttggccgtgtcggcctcgctcaactggaccttgccggccgccgcgtccgcgaggcgcttttcagcgtgcaaatg
gttgcggtagttctccggggtgatcggcggcagctccttcgggtaggcgttctcgcccggcgcgaagattgggaagatggccttgccgc
cgaccgccttggcggcctcctgtgccttcgtcctgccgggattcacgccctgggtgatctgcacctggcggtcgtcgtcgccggcgatc
acaacgggcttgtccgggaatttcgcgtgcagggcctcggcaacagcctgtaggttgccggaatcgaacgcggcgacagtcgcgtgccc
cagcgcttcggccactgtggcggcggtggcatagccttcgccgatcaccagcgccggcgcggccgcgagcgcatccatgccaccgacga
catggaagcatccttccttgcggctgtccttggcgaagcgcttggtgccgtcctcctggatgtactgcatggtccattgcttgccgtcg
gcgtcgtaggccgggatgtaggttttctggccctcctggtcggtaaggacgccggcgtgcacctgtagacccttgtcgcgcaggtacgg
cgtcggttccgtgatgggaaccaggctttgcgcctggcggccgatgcgctgcgccgtggcttcgtgctggcgttcttgttcctcggcac
gcgcggccagcttggccgccgcctcggcctgcatcttggccttctcggcggggtccagggcgtagcccttggccttccacttcatttcg
acgccggtgcggttgtttttgatgtaaccggccgggtggccgtcgaggtggccgacgtagaagcccgacttctcgcccttcttgtcgcc
ctcggtctcgatgcggtgcttcttgccgtccatgatggggtgctcgccgcctggggtgacgacgcagcccatgatttcagggcctccgc
gaactcatcttcgggggtgacggccggggattgctgggtgggcacgttgtccggcagccagcgttgcagcttgcccatgtcggcgttcg
gtccggcgtaccaggacttggccaccttgtcccactgcgcgccggccgccttggcaacctggcgctcgccgtagggcacggccaggtag
acgcgctcctgggccgcgttggggcgctcggccgtgggttgggtaggctgggcctcggcgcgggcctctacggccgctgtagcgccctc
gcgcgcccatttggcgaacggggcagggtcaacccctgccggaacgtaccaggcgcgttcctggcggtcccagcgcgctccaagggcct
tcacctcgtctttctccttgaacggcacgttcaagtaggcgcgctcgggcttggcgggggcttgagcggccgccggctgctcggcggcg
ttcatggcctgggccatttcctgctgctcgcgctcgtagtcggcgatccggcgctgtaggtcctcgtcgtgcagcatggccgtgccctc
ggcggccttgcgcgcctccttggcggcaacgcggtcctcgtcggtgctgttgggatcgcggcgaactcgctcttcatggatgcgggcga
acttcgcggcctgctcgtactcgttggccgtcgcgtaagcgtcgatcacggccaggcggtcggcgagcgcttcggcattggtctgcgcg
tcggggccggcgaagtcggcaagccattggtggccgccccacgcatggttcgcatagacgccccaaaactccggctctcggtcgccggc
cggcaccacggaccgttcgccgtcgtgctcgacctcgacgttggcctggacctggacgcggccggtccaatcggcaggcagctcaaagc
ccagcgtggtttcggtcagcgcggccagcgattggttgccttccgccggctccgcgccggcgcggtacatgcgcagggtctgcgcgatc
agctcgtcggccggcgcaatggccggtcgtgccacctggtcttgctgttgctccatagttgccccctgcgcaggctcgatggcctgctg
ggtcgtttgttcttgaatttgcttctgctcgaacgccaggacgaaatcctggatcttctccgcgtcggcggccgcgcggaaaatctcta
gcgggtcctcttgtagcgccttgatccacgatccgacataggccgcgtgctggccggggtcgtggccgatgcccagctcgtcgcccagg
atcatgctggcaatctcggcccgcagctcttccttggcgtacccctcgctcccgaagggatgcgccaggtcgcggtccagccgcgacgg
gtggccggtccagtgccccagctcatggagcgcggttgcgtagtagttgtcggcgctcgggaactggcctttgtcgggcagatggatgc
tgtccgtggacggccgataaaacgcgcggtcgtgctcgccgtggcggatggtggcacctgacgccgcaaggatgtgctcggcccgctcg
acggcgctccaagtctgttccttgcgttccaacggcggcaggccgtcgatctgctccgcattgaacacggtggcgaagaacacgcgcgg
gcgttcgagctgcaccgtcaccttgaccggatcgccgttggcatcgaggaccggcttgccggtctgctcgtcggtcttggtctgctctt
cgctgaacttccaatactggatcggcgtgcctttctcgccgcgacgcacctgtgcgccggcggcagcggcctgcttgtaggtcatccag
cgcgggtccgcatggccctgggccatgagctgaatcgcgttgatgcccttgtaacgcttcccggtagtcgggttgagcgggatgaagga
gccgggcatgcccggttcccacggtttttgccacggcgcagtgccggctttcagttgctcaatgaggcgttcggcaacctgctcgtgga
acggctttttgacctctgccatagccaattacctcccgtcattggcggccgcggtcgtcgtgtcctcgggcacggtcgcgtccaccagg
tcaatgtcgctctcggcggcgtcctgctcgctcaaggcgtcctcggggaaggccccggccttctccgcttcttccgggtcgaactcgac
ctggaagccgggcgtcatcgcatcggcgagcttttcacgcagggcggcggcggcgttcgcggtatcgtcgaacggcgcaaagtcgtcct
gctgcacggtcttgggatcattcatcgctttcactcctggttggtgccgttacggcctttgctgtagtccggcctgcctttcaggtcgg
ggtatgtctgcttgcacgtcgggaagttgctgcacccccaccagaacatgccgcgcttcttgccaggccgacgggaaaggccgtggccg
caggccatgcacttgtgcagctcggagactttcggggcctcgcgcgggacgggcttgccgcccttgtcgtcgcacgcgaacttgcagcc
gtcggcaaagccggtgcagccccaaaagtattcgttcttgtccttcttcttgaggcgtcgcagcggcttgccgcaggacgggcaagggt
gcgtgtcgatcttcatgttgaggccgttgtattgatgttggcgacctcggcgccgatgtattccatcagctcgttgacgaacgacagcg
tgtcgcgctcgccggcctggatggccttctgctgctcatgccagagcgcggtcatgtcggggaatctggccgtgtcgggcagtgcgtcg
tacagctcttcgccggtcggcgtggacacgatgtgcttgcccttctccaccaggtagccgcgctcgaaaagcgtggcgatgatggagtc
tcgcgttgccggcgtgccgatcccgccgtgctcgccttgcttgcccttgtccttttcgatcaagattttccgcaggcggtcatcgcgga
tgtatttcgcaacgcgggtaaggtccgacagcagagattccatcgtgtacagcggctgcggtttcgtctcctgctgctcggccttcgca
tcggtgcaggtgccggcctggccgtcacgcagcttgcgcaggtcctgttcaatgtcgtcggcattgccttccaggtcctcgttgccggc
gtcgttcttgtagagaatcttccagcccggcgacgtggtgacgttcgagcgcacgccgaaacgatgatcgccgacctgggcaagcacgt
cggtctggtcatacagatgcttcggccagaactgcgcgacgtaggcgcgcgcgatcagcaggtaaatcttctgctcggcatcggtgagc
ttcgacaggtcggccgtgctttcggtcgggatgatcgcgtggtgcgcggaaaccttggacgagttgaaggcgcggctcttgatcgtcgg
attggcgcgctgcgcagcagcggccagcatgggggccgtctgtgcgatggccgccagcacgcccggcgcatcgccgtgctgttcctcgc
tcaagtattcgcagtcggaacggttgtaggtgatgagcttgtgcttctcgcgcagggcctgcgtaatgtccttcacctggtccggcttg
aagccgaacttgcgcgaggcgtccatttgcagtttcagcaggttgtagggcagcggcgcagccgcttccttcgccttggtggtcacgga
cacgatgcgggcgggttggccgctcacggcggccgcgatgccctcggcgtgctccttgttgctgaggcggcctttctcgtccaccggat
cgccgtcggcgacctggtaacgggccgggaactgaatgccctcgacctcgaactggccgttcaccaggtagtagtaggttttctggtgg
gccgcgttctcgcggcaacggcgcacgacaaggcccaggatcggagtctgcacgcgccccacgctcaacagcccctgatagcccttcgc
gcgtgccgcaagcgtgtacaggcgcgtgatgttgaagccgtatagctggtcgccgacgctgcgggcctcggccgcagcggacaggccgg
cgaactcgcggttgtcgcgcatcgcggcgagctgccggcgcacgatcttcacgttgttgtcgttgataagcagccgctgcaccggcaga
cggcagttggcgtattccaggatttcatcgaccagaagctggccttcgtcgtccgggtcgccggcgtgaaccacgcttttcgcctgctt
caacaggctgaggatggtcttgaactgagctttcgcaccggcatcgccggacggtttcttgcgccagggaatatggacgatgggcaggt
cggccatgttccagttggcgtagcgctcgtcgtagtcctccgggtctagcaaggccagcatgtgaccgtagcaccaggtcacgcggtcg
gagccgcattcgtaatagccgtccttgcggctgccgccgcccaggccctcgacgatggcttttgccagctccggtttttcagcgattac
aaggcgttcaaattgcatatatccccctaccctcaccaggtcagaaccggcctgatgacggtgatgatttgcgaacgattgacaggccc
gaagtagcggccgtcgaaagacgtgtcgcttacgtcggacataagcagaacctcggcggtccccagggtgtagctgtcggactgataac
gaggcagcggccgtcctgatggatcggccttgatgagcgcgctgtgaggcagcagcccgccattcacgcgcacgccggcgtcggtgatg
gcaacctcgtcgcctttagcggctaaaactcgcttcatcatgtagccgtagtcgccggggcagaaaccgccggcgatgtagccccgctc
cttggcgtccgaaaacacgccgacttgcggcgggcagaacatgacgtaagcccccttctccaccggcgcattcgatttccagtacaggc
cgaccggaatgcttttggtggtgttgaccttcgcgccggcgagataggccgcgccggcgagcaacaaggccgcgccgcctccgatggcg
acgtacttggtgaggcgctggaagcggctcatatcgtgatcccctccccttcctcgacggtggccgtctggatcagcttgtcgctgacc
ttcggagccggtacggccgcgcgggcctggaatatcgggtctttgaagtagagcggctgcttgccgtagatcgcgggatagccggcgac
gtacacaaccatgtcgcccgcctcttcaatgctgccgtcggcgctcttcttcggccccggcatgcgcaggcattcatcgggggtcagca
atggccgctgcacttcctggaaggtccgcgagacgttgcccaacagcgccgacgtgcggcggccgctcgtcgtgatctgctccttcacg
atggtcgtggtgcctgtcagttttgacaggtgctcggccgtctccacgcggttcggcgggtaggcgttctgcacgtggcagttcgacgt
gatgctttcgtcgtggccgtagccggtttcgcggctcttgagctggttaatgtcctggcagatgaggtagcacttgatgccgtagccgg
cgacgaaggcaagggactcttgcaggatttcgagcttgcccaggctggggaactcgtcgagcatcatcagcagacgatgcttgtagtgc
gcgacaggacggccgttctcgaagtccatcttgtcggccagcagccggacgatcatgttgaccatgacgcgcaccagaggccgcagacg
ggccttgtcgttgggctgcgtcacgatgaacaggcttaccgggtcgtcgtggtgcatcagttgcttgatgcggaagtcggacttgctga
cgttgcgggccacaaccgggtcgcggtacagggccaggtaggacttggcggtggacagcacggaaccggattcttcctccgggcggtcc
atcatgtcgcgggccgcagagccgaccgcagggtggttctgcccgtcaacgtggccgtaggtggtcatttccatccaaagctcgcccac
gtcgcggttcgggtcggcaagcatgccgtccaccgacggcagggtggccggcgtaccctcgttcttagccttgtagagcgcgtgcagga
tgacgccgacaagcagcgcctggctggttttctgccagtgcgattccaggcccttgccgtccggatcgacgatcagggtggcaaggttc
tgcacgtcgccaacctcgtactcggtccccaagcggatttcatcgagcgggttccagcacgcgctaccctgcgcggatgccggctcaaa
gcgcacgaccttgttgcgggcatgcttcttccgccagccggcggtcagcgcccacaactcgcctttcaggtcggtgatgacggcgctgt
gcgcccaggaaagcagcgtcggaacgaccaggccgacgcccttgccggagcgcgtcggcgcgtaggtcaagacgtgctcggggccgttg
tgccgcaggtagtggaacttgccgtccttgtcctgccagccgcccacatagacgccgctggaagtgggcgggtgtttgcctgacaccag
ctcgacgacggtgcgcggccggggcagcaggccggcggcctgtatgtccttcttgtcggcccagcgggccgaaccgtgcagatagtcgt
tcgccttgccggtgttcgccttgaccatctgcgtgacggccgtgcccagcaggcccacggtcgaaacgaccatacccatgctggccgcg
cgcatgaaatcgtcgggatattggccgtaccacttgccggcccattgaaggatcgaccagggcgtgtagacgtggttgatattccagcc
aagtccggcctgatactggaaggaatgggcgaaatattgcgtcgcggtctgcaagcctgccccaagggacaggccggcgaggatgggaa
cggtcttgctggccttcggttttttcgcccgtatctgtggccccacggcgttgtttcggttcttcatctactcctacctcgggtagttt
taagggagcctcgcggggtcacggtgacgggatcaccgatggcgaggcgcttcatgcgttgcaccgtggccttatcgacgggcagcacc
agaatctcgtcgttttctttcctcaacagggccagcgcctggtcctcgacgttccgggtgcctgcataggacagcgcaccaacataatc
agtatatcgtgcatgcttcggtatatcgaagccgtttagccgcttttgctcgcgctcggcaacatatttctcggccgccgcgatctgtt
cgggctttagccctcttcctggcccagaaactccccgtcgcagtgcgtgagctggttcggctccttgctgctccacgtgaccaggaaca
tcacgcggcaatagcatttcagctccgccggcgatgcgaaccacaccgagttgggacagcgctcgcaaacggttttggctttggggcgg
cggctttcgtccaatgcgtccaacgttgggcttgcggagtgcgacggttccgccggcgctgacggcgcgagcgtcccgtcggtcgccgt
cgccgcctgtggcgttgagggtggttctggctgcggcaggtcgaatgcctccatcgccgccgcgatctcttcgtccgtcatttcgttcg
ggttgctcatgtgcttgctccttcgtcagtagttcttgacggcggcgctcaagggcggcgtcgtcaaaggtgattgccagacggccagc
ggcggccgcctgcgcgatccgctccttgaactctgctgtgccgttgacggtgatccggtcgccgaagcgctccattgccaggcgcaggg
cggcgtccaggccgtccgtggtggcctcgcgcgagacttgcaggcggtcgccgtcgtcgcggacggcgctgctgccgacgcgatagatg
atggttcccttcttcgtgatgttgtccgtcacggccgcatggcccggcttggcctcgccgctgccctggatggtgttgcccttgaggtc
gctgcggccctcgcgtgcgcgcagcgcggccagggccttgtcgtcgcccttcatcgcctcggccttgagccagtcggcccacgcgcggc
gctgcgtgcgctcctggaccgcctgacggccctgccggtactcgcggttgatcttgtccaggtcggcgcgcagagccttgtgcgcctgc
gcgtacatcagtcgctttgcaatgcgcccctcgcccagcagcttgatagcggcgcggcgcagccggttgctgcgcatcgcggcttcaat
caggcggtcacgacgccggcgcagcgtgtccagctcgcccttgcgcacggcccccatttcctggcgttcagactgataccgggcgtata
gctcggtggtgtcgatgcgggtcttgagcggcttcgctcgatactcccgccgccggggggcttcgccgccctcggctggcgtgaatgcc
ccgaatcgggcttcgagcttcggcttggacaggtcgcgcgaaacggtgctggccttgaccgtcgtgccgtcgccggcctcgaagatgaa
gccgtttccgcgctcgcgcagcttaagcccgttttcccgcaggacgcggtgcaggtcctcccaggattgcgccgcttgcagctccggca
ggcattcgcgcttgatccagccgaccaggctttccacgcccgcgtgccgctccatgtcgttcgcgcggttctcggaaacgcgctgccgc
gtttcgtgattgtcacgctcaagcccgtagtcccgttcgagcgtcgcgcagaggtcagcgagggcgcggtaggcccgatacggctcatg
gatggtgtttcgggtcgggtgaatcttgttgatggcgatatggatgtgcaggttgtcggtgtcgtgatgcacggcactgacgcgctgat
gctcggcgaagccaagcccagcgcagatgcggtcctcaatcgcgcgcaacgtctccgcgtcgggcttctctcccgcgcggaagctaacc
agcaggtgataggtcttgtcggcctcggaacgggtgttgccgtgctgggtcgccatcacctcggccatgacagcgggcagggtgtttgc
ctcgcagttcgtgacgcgcacgtgacccaggcgctcggtcttgccttgctcgtcggtgatgtacttcaccagctccgcgaagtcgctct
tcttgatggagcgcatggggacgtgcttggcaatcacgcgcaccccccggccgttttagcggctaaaaaagtcatggctctgccctcgg
gcggaccacgcccatcatgaccttgccaagctcgtcctgcttctcttcgatcttcgccagcagggcgaggatcgtggcatcaccgaacc
gcgccgtgcgcgggtcgtcggtgagccagagtttcagcaggccgcccaggcggcccaggtcgccattgatgcgggccagctcgcggacg
tgctcatagtccacgacgcccgtgattttgtagccctggccgacggccagcaggtaggccgacaggctcatgccggccgccgccgcctt
ttcctcaatctctcttcgttcgtctggaaggcagtacaccttgataggtgggctgcccttcctggttggcttggtttcatcagccatcc
gcttgccgaattctgacgccgttggatacaccaaggaaagtctacacgaaccctttggcaaaatcctgtatatcgtgcgaaaaaggatg
gatataccgaaaaaatcgctataatgaccccgaagcagggttatgcagcggaaaagcgctgcttccctgctgttttgtggaatatctac
cgactggaaacaggcaaatgcaggaaattactgaactgaggggacaggcgagagacgatgccaaagagctacaccgacgagctggccga
gtgggttgaatcccgcgcggccaagaagcgccggcgtgatgaggctgcggttgcgttcctggcggtgagggcggatgtcgaggcggcgt
tagcgtccggctatgcgctcgtcaccatttgggagcacatgcgggaaacggggaaggtcaagttctcctacgagacgttccgctcgcac
gccaggcggcacatcaaggccaagcccgccgatgtgcccgcaccgcaggccaaggctgcggaacccgcgccggcacccaagacgccgga
gccacggcggccgaagcaggggggcaaggctgaaaagccggcccccgctgcggccccgaccggcttcaccttcaacccaacaccggaca
aaaaggatctactgtaatggcgaaaattcacatggttttgcagggcaagggcggggtcggcaagtcggccatcgccgcgatcattgcgc
agtacaagatggacaaggggcagacacccttgtgcatcgacaccgacccggtgaacgcgacgttcgagggctacaaggccctgaacgtc
cgccggctgaacatcatggccggcgacgaaattaactcgcgcaacttcgacaccctggtcgagctgattgcgccgaccaaggatgacgt
ggtgatcgacaacggtgccagctcgttcgtgcctctgtcgcattacctcatcagcaaccaggtgccggctctgctgcaagaaatggggc
atgagctggtcatccataccgtcgtcaccggcggccaggctctcctggacacggtgagcggcttcgcccagctcgccagccagttcccg
gccgaagcgcttttcgtggtctggctgaacccgtattgggggcctatcgagcatgagggcaagagctttgagcagatgaaggcgtacac
ggccaacaaggcccgcgtgtcgtccatcatccagattccggccctcaaggaagaaacctacggccgcgatttcagcgacatgctgcaag
agcggctgacgttcgaccaggcgctggccgatgaatcgctcacgatcatgacgcggcaacgcctcaagatcgtgcggcgcggcctgttt
gaacagctcgacgcggcggccgtgctatgagcgaccagattgaagagctgatccgggagattgcggccaagcacggcatcgccgtcggc
cgcgacgacccggtgctgatcctgcataccatcaacgcccggctcatggccgacagtgcggccaagcaagaggaaatccttgccgcgtt
caaggaagagctggaagggatcgcccatcgttggggcgaggacgccaaggccaaagcggagcggatgctgaacgcggccctggcggcca
gcaaggacgcaatggcgaaggtaatgaaggacagcgccgcgcaggcggccgaagcgatccgcagggaaatcgacgacggccttggccgc
cagctcgcggccaaggtcgcggacgcgcggcgcgtggcgatgatgaacatgatcgccggcggcatggtgttgttcgcggccgccctggt
ggtgtgggcctcgttatgaatcgcagaggcgcagatgaaaaagcccggcgttgccgggctttgtttttgcgttagctgggcttgtttga
caggcccaagctctgactgcgcccgcgctcgcgctcctgggcctgtttcttctcctgctcctgcttgcgcatcagggcctggtgccgtc
gggctgcttcacgcatcgaatcccagtcgccggccagctcgggatgctccgcgcgcatcttgcgcgtcgccagttcctcgatcttgggc
gcgtgaatgcccatgccttccttgatttcgcgcaccatgtccagccgcgtgtgcagggtctgcaagcgggcttgctgttgggcctgctg
ctgctgccaggcggcctttgtacgcggcagggacagcaagccgggggcattggactgtagctgctgcaaacgcgcctgctgacggtcta
cgagctgttctaggcggtcctcgatgcgctccacctggtcatgctttgcctgcacgtagagcgcaagggtctgctggtaggtctgctcg
atgggcgcggattctaagagggcctgctgttccgtctcggcctcctgggccgcctgtagcaaatcctcgccgctgttgccgctggactg
attactgccggggactgctgttgccctgctcgcgccgtcgtcgcagttcggcttgcccccactcgattgactgcttcatttcgagccgc
agcgatgcgatctcggattgcgtcaacggacggggcagcgcggaggtgtccggcttctccttgggtgagtcggtcgatgccatagccaa
aggtttccttccaaaatgcgtccattgctggaccgtgtttctcattgatgcccgcaagcatcttcggcttgaccgccaggtcaagcgcg
ccttcatgggcggtcatgacggacgccgccatgaccttgccgccgttgttctcgatgtagccgcgtaatgaggcaatggtgccgcccat
cgtcagcgtgtcatcgacaacgatgtacttctggccggggatcacctccccctcgaaagtcgggttgaacgccaggcgatgatctgaac
cggctccggttcgggcgaccttctcccgctgcacaatgtccgtttcgacctcaaggccaaggcggtcggccagaacgaccgccatcatg
gccggaatcttgttgttccccgccgcctcgacggcgaggactggaacgatgcggggcttgtcgtcgccgatcagcgtcttgagctgggc
aacagtgtcgtccgaaatcaggcgctcgaccaaattaagcgccgcttccgcgtcgccctgcttcgcagcctggtattcaggctcgttgg
tcaaagaaccaaggtcgccgttgcgaaccaccttcgggaagtctccccacggtgcgcgctcggctctgctgtagctgctcaagacgcct
ccctttttagccgctaaaactctaacgagtgcgcccgcgactcaacttgacgctttcggcacttacctgtgccttgccacttgcgtcat
aggtgatgcttttcgcactcccgatttcaggtactttatcgaaatctgaccgggcgtgcattacaaagttcttccccacctgttggtaa
atgctgccgctatctgcgtggacgatgctgccgtcgtggcgctgcgacttatcggccttttgggccatatagatgttgtaaatgccagg
tttcagggccccggctttatctaccttctggttcgtccatgcgccttggttctcggtctggacaattctttgcccattcatgaccagga
ggcggtgtttcattgggtgactcctgacggttgcctctggtgttaaacgtgtcctggtcgcttgccggctaaaaaaaagccgacctcgg
cagttcgaggccggctttccctagagccgggcgcgtcaaggttgttccatctattttagtgaactgcgttcgatttatcagttactttc
ctcccgctttgtgtttcctcccactcgtttccgcgtctagccgacccctcaacatagcggcctcttcttgggctgcctttgcctcttgc
cgcgcttcgtcacgctcggcttgcaccgtcgtaaagcgctcggcctgcctggccgcctcttgcgccgccaacttcctttgctcctggtg
ggcctcggcgtcggcctgcgccttcgctttcaccgctgccaactccgtgcgcaaactctccgcttcgcgcctggtggcgtcgcgctcgc
cgcgaagcgcctgcatttcctggttggccgcgtccagggtcttgcggctctcttctttgaatgcgcgggcgtcctggtgagcgtagtcc
agctcggcgcgcagctcctgcgctcgacgctccacctcgtcggcccgctgcgtcgccagcgcggcccgctgctcggctcctgccagggc
ggtgcgtgcttcggccagggcttgccgctggcgtgcggccagctcggccgcctcggcggcctgctgctctagcaatgtaacgcgcgcct
gggcttcttccagctcgcgggcctgcgcctcgaaggcgtcggccagctccccgcgcacggcttccaactcgttgcgctcacgatcccag
ccggcttgcgctgcctgcaacgattcattggcaagggcctgggcggcttgccagagggcggccacggcctggttgccggcctgctgcac
cgcgtccggcacctggactgccagcggggcggcctgcgccgtgcgctggcgtcgccattcgcgcatgccggcgctggcgtcgttcatgt
tgacgcgggcggccttacgcactgcatccacggtcgggaagttctcccggtcgccttgctcgaacagctcgtccgcagccgcaaaaatg
cggtcgcgcgtctctttgttcagttccatgttggctccggtaattggtaagaataataatactcttacctaccttatcagcgcaagagt
ttagctgaacagttctcgacttaacggcaggttttttagcggctgaagggcaggcaaaaaaagccccgcacggtcggcgggggcaaagg
gtcagcgggaaggggattagcgggcgtcgggcttcttcatgcgtcggggccgcgcttcttgggatggagcacgacgaagcgcgcacgcg
catcgtcctcggccctatcggcccgcgtcgcggtcaggaacttgtcgcgcgctaggtcctccctggtgggcaccaggggcatgaactcg
gcctgctcgatgtaggtccactccatgaccgcatcgcagtcgaggccgcgttccttcaccgtctcttgcaggtcgcggtacgcccgctc
gttgagcggctggtaacgggccaattggtcgtaaatggctgtcggccatgagcggcctttcctgttgagccagcagccgacgacgaagc
cggcaatgcaggcccctggcacaaccaggccgacgccgggggcaggggatggcagcagctcgccaaccaggaaccccgccgcgatgatg
ccgatgccggtcaaccagcccttgaaactatccggccccgaaacacccctgcgcattgcctggatgctgcgccggatagcttgcaacat
caggagccgtttcttttgttcgtcagtcatggtccgccctcaccagttgttcgtatcggtgtcggacgaactgaaatcgcaagagctgc
cggtatcggtccagccgctgtccgtgtcgctgctgccgaagcacggcgaggggtccgcgaacgccgcagacggcgtatccggccgcagc
gcatcgcccagcatggccccggtcagcgagccgccggccaggtagcccagcatggtgctgttggtcgccccggccaccagggccgacgt
gacgaaatcgccgtcattccctctggattgttcgctgctcggcggggcagtgcgccgcgccggcggcgtcgtggatggctcgggttggc
tggcctgcgacggccggcgaaaggtgcgcagcagctcgttatcgaccggctgcggcgtcggggccgccgccttgcgctgcggtcggtgt
tccttcttcggctcgcgcagcttgaacagcatgatcgcggaaaccagcagcaacgccgcgcctacgcctcccgcgatgtagaacagcat
cggattcattcttcggtcctccttgtagcggaaccgttgtctgtgcggcgcgggtggcccgcgccgctgtctttggggatcagccctcg
atgagcgcgaccagtttcacgtcggcaaggttcgcctcgaactcctggccgtcgtcctcgtacttcaaccaggcatagccttccgccgg
cggccgacggttgaggataaggcgggcagggcgctcgtcgtgctcgacctggacgatggcctttttcagcttgtccgggtccggctcct
tcgcgcccttttccttggcgtccttaccgtcctggtcgccgtcctcgccgtcctggccgtcgccggcctccgcgtcacgctcggcatca
gtctggccgttgaaggcatcgacggtgttgggatcgcggcccttctcgtccaggaactcgcgcagcagcttgaccgtgccgcgcgtgat
ttcctgggtgtcgtcgtcaagccacgcctcgacttcctccgggcgcttcttgaaggccgtcaccagctcgttcaccacggtcacgtcgc
gcacgcggccggtgttgaacgcatcggcgatcttctccggcaggtccagcagcgtgacgtgctgggtgatgaacgccggcgacttgccg
atttccttggcgatatcgcctttcttcttgcccttcgccagctcgcggccaatgaagtcggcaatttcgcgcggggtcagctcgttgcg
ttgcaggttctcgataacctggtcggcttcgttgtagtcgttgtcgatgaacgccgggatggacttcttgccggcccacttcgagccac
ggtagcggcgggcgccgtgattgatgatatagcggcccggctgctcctggttctcgcgcaccgaaatgggtgacttcaccccgcgctct
ttgatcgtggcaccgatttccgcgatgctctccggggaaaagccggggttgtcggccgtccgcggctgatgcggatcttcgtcgatcag
gtccaggtccagctcgatagggccggaaccgccctgagacgccgcaggagcgtccaggaggctcgacaggtcgccgatgctatccaacc
ccaggccggacggctgcgccgcgcctgcggcttcctgagcggccgcagcggtgtttttcttggtggtcttggcttgagccgcagtcatt
gggaaatctccatcttcgtgaacacgtaatcagccagggcgcgaacctctttcgatgccttgcgcgcggccgttttcttgatcttccag
accggcacaccggatgcgagggcatcggcgatgctgctgcgcaggccaacggtggccggaatcatcatcttggggtacgcggccagcag
ctcggcttggtggcgcgcgtggcgcggattccgcgcatcgaccttgctgggcaccatgccaaggaattgcagcttggcgttcttctggc
gcacgttcgcaatggtcgtgaccatcttcttgatgccctggatgctgtacgcctcaagctcgatgggggacagcacatagtcggccgcg
aagagggcggccgccaggccgacgccaagggtcggggccgtgtcgatcaggcacacgtcgaagccttggttcgccagggccttgatgtt
cgccccgaacagctcgcgggcgtcgtccagcgacagccgttcggcgttcgccagtaccgggttggactcgatgagggcgaggcgcgcgg
cctggccgtcgccggctgcgggtgcggtttcggtccagccgccggcagggacagcgccgaacagcttgcttgcatgcaggccggtagca
aagtccttgagcgtgtaggacgcattgccctgggggtccaggtcgatcacggcaacccgcaagccgcgctcgaaaaagtcgaaggcaag
atgcacaagggtcgaagtcttgccgacgccgcctttctggttggccgtgaccaaagttttcatcgtttggtttcctgttttttcttggc
gtccgcttcccacttccggacgatgtacgcctgatgttccggcagaaccgccgttacccgcgcgtacccctcgggcaagttcttgtcct
cgaacgcggcccacacgcgatgcaccgcttgcgacactgcgcccctggtcagtcccagcgacgttgcgaacgtcgcctgtggcttccca
tcgactaagacgccccgcgctatctcgatggtctgctgccccacttccagcccctggatcgcctcctggaactggctttcggtaagccg
tttcttcatggataacacccataatttgctccgcgccttggttgaacatagcggtgacagccgccagcacatgagagaagtttagctaa
acatttctcgcacgtcaacacctttagccgctaaaactcgtccttggcgtaacaaaacaaaagcccggaaaccgggctttcgtctcttg
ccgcttatggctctgcacccggctccatcaccaacaggtcgcgcacgcgcttcactcggttgcggatcgacactgccagcccaacaaag
ccggttgccgccgccgccaggatcgcgccgatgatgccggccacaccggccatcgcccaccaggtcgccgccttccggttccattcctg
ctggtactgcttcgcaatgctggacctcggctcaccataggctgaccgctcgatggcgtatgccgcttctccccttggcgtaaaaccca
gcgccgcaggcggcattgccatgctgcccgccgctttcccgaccacgacgcgcgcaccaggcttgcggtccagaccttcggccacggcg
agctgcgcaaggacataatcagccgccgacttggctccacgcgcctcgatcagctcttgcactcgcgcgaaatccttggcctccacggc
cgccatgaatcgcgcacgcggcgaaggctccgcagggccggcgtcgtgatcgccgccgagaatgcccttcaccaagttcgacgacacga
aaatcatgctgacggctatcaccatcatgcagacggatcgcacgaacccgctgaa

TABLE 4
Sequence of pNuc-trans (SEQ ID NO: 67)
acgaccgggtcgaatttgattcgaatttctgccattcatccgcttattatcacttattcaggcgtagcaaccaggcgtttaagggcacc
aataactgccttaaaaaaattacgccccgccctgccactcatcgcagtactgttgtaattcattaagcattctgccgacatggaagcca
tcacaaacggcatgatgaacctgaatcgccagcggcatcagcaccttgtcgccttgcgtataatatttgcccatagtgaaaacgggggc
gaagaagttgtccatattggccacgtttaaatcaaaactggtgaaactcacccagggattggctgagacgaaaaacatattctcaataa
accattagggaaataggccaggttttcaccgtaacacgccacatcttgcgaatatatgtgtagaaactgccggaaatcgtcgtggtatt
cactccagagcgatgaaaacgtttcagtttgctcatggaaaacggtgtaacaagggtgaacactatcccatatcaccagctcaccgtct
ttcattgccatacggaactccggatgagcattcatcaggcgggcaagaatgtgaataaaggccggataaaacttgtgcttatttttctt
tacggtctttaaaaaggccgtaatatccagctgaacggtaggttataggtacattgagcaactgactgaaatgcctcaaaatgttatta
cgatgccattgggatatatcaacggtggtatatccagtgatttttttaccattttagcttccttagctcctgaaaatctcgataactca
aaaaatacgcccggtagtgatcttatttcattatggtgaaagttggaacctcttacgtgccgatcaacgtctcattttcgccaaaagtt
ggcccagggcttcccggtatcaacagggacaccaggatttatttattctgcgaagtgatcttccgtcacaggtatttattcggcgcaaa
gtgcgtcgggtgatgctgccaacttactgatttagtgtatgatggtgtttttgaggtgctccagtggcttctgtttctatcagctgtcc
ctcctgttcagctactgacggggtggtgcgtaacggcaaaagcaccgccggacatcagcgctagcggagtgtatactggcttactatgt
tggcactgatgagggtgtcagtgaagtgcttcatgtggcaggagaaaaaaggctgcaccggtgcgtcagcagaatatgtgatacaggat
atattccgcttcctcgctcactgactcgctacgctcggtcgttcgactgcggcgagcggaaatggcttacgaacggggcggagatttcc
tggaagatgccaggaagatacttaacagggaagtgagagggccgcggcaaagccgtttttccataggctccgcccccctgacaagcatc
acgaaatctgacgctcaaatcagtggtggcgaaacccgacaggactataaagataccaggcgtttccccctggcggctccctcgtgcgc
tctcctgttcctgcctttcggtttaccggtgtcattccgctgttatggccgcgtttgtctcattccacgcctgacactcagttccgggt
aggcagttcgctccaagctggactgtatgcacgaaccccccgttcagtccgaccgctgcgccttatccggtaactatcgtcttgagtcc
aacccggaaagacatgcaaaagcaccactggcagcagccactggtaattgatttagaggagttagtcttgaagtcatgcgccggttaag
gctaaactgaaaggacaagttttggtgactgcgctcctccaagccagttacctcggttcaaagagttggtagctcagagaaccttcgaa
aaaccgccctgcaaggcggttttttcgttttcagagcaagagattacgcgcagaccaaaacgatctcaagaagatcatcttattaatca
gataaaatatttctagatttcagtgcaatttatctcttcaaatgtagcacctgaagtcagccccatacgatataagttgtaattctcat
gttagtcatgccccgcgcccaccggaaggagctgactgggttgaaggctctcaagggcatcggtcgagatcccggtgcctaatgagtga
gctaacttacattaattgcgttgcgcgatcgtcttgccttgctcgtcggtgatgtacttacagctcgaagtgcctcttcttgatggagc
gcatggggacgtgcttggcaatcacgcgcaccccccggccgttttagcggctaaaaaagtcatggctctgccctcgggcggaccacgcc
catcatgaccttgccaagctcgtcctgcttctcttcgatcttcgccagcagggcgaggatcgtggcatcaccgaaccgcgccgtgcgcg
ggtcgtcggtgagccagagtttcagcaggccgcccaggcggcccaggtcgccattgatgcgggccagctcgcggacgtgctcatagtcc
acgacgcccgtgattttgtagccctggccgacggccagcaggtaggccgacaggctcatgccggccgccgccgccttttcctcaatcgc
tcttcgttcgtctggaaggcagtacaccttgataggtgggctgcccttcctggttggcttggtttcatcagccatccgcttgccctcat
ctgttacgccggcggtagccggccagcctcgcagagcaggattcccgttgagcaccgccaggtgcgaataagggacagtgaagaaggaa
cacccgctcgcgggtgggcctacttcacctatcctgcccggctgacgccgttggatacaccaaggaaagtctacacgaaccctttggca
aaatcctgtatatcgtgcgaaaaaggatggatataccgaaaaaatcgctataatgaccccgaagcagggttatgcagcggaagatatcg
atgcataatgtgcctgtcaaatggacgaagcagggattctgcaaaccctatgctactccgtcaagccgtcaattgtctgattcgttacc
aattatgacaacttgacggctacatcattcactttttcttcacaaccggcacggaactcgctcgggctggccccggtgcattttttaaa
tacccgcgagaaatagagttgatcgtcaaaaccaacattgcgaccgacggtggcgataggcatccgggtggtgctcaaaagcagcttcg
cctggctgatacgttggtcctcgcgccagcttaagacgctaatccctaactgctggcggaaaagatgtgacagacgcgacggcgacaag
caaacatgctgtgcgacgctggcgatatcaaaattgctgtctgccaggtgatcgctgatgtactgacaagcctcgcgtacccgattatc
catcggtggatggagcgactcgttaatcgcttccatgcgccgcagtaacaattgctcaagcagatttatcgccagcagctccgaatagc
gcccttccccttgcccggcgttaatgatttgcccaaacaggtcgctgaaatgcggctggtgcgcttcatccgggcgaaagaaccccgta
ttggcaaatattgacggccagttaagccattcatgccagtaggcgcgcggacgaaagtaaacccactggtgataccattcgcgagcctc
cggatgacgaccgtagtgatgaatctctcctggcgggaacagcaaaatatcacccggtcggcaaacaaattctcgtccctgatttttca
ccaccccctgaccgcgaatggtgagattgagaatataacctttcattcccagcggtcggtcgataaaaaaatcgagataaccgttggcc
tcaatcggcgttaaacccgccaccagatgggcattaaacgagtatcccggcagcaggggatcattttgcgcttcagccatacttttcat
actcccgccattcagagaagaaaccaattgtccatattgcatcagacattgccgtcactgcgtcttttactggctcttctcgctaacca
aaccggtaaccccgcttattaaaagcattctgtaacaaagcgggaccaaagccatgacaaaaacgcgtaacaaaagtgtctataatcac
ggcagaaaagtccacattgattatttgcacggcgtcacactttgctatgccatagcatttttatccataagattagcggatcctacctg
acgctttttatcgcaactctctactgtttctccatacccgtttttttgggctagccctgtagaaataattgtttaactttaataaggag
atataccatgggtaaaagcggaatttatcagattaaaaatactttaaacaataaagtatatgtaggaagtgctaaagattttgaaaaga
gatggaagaggcattttaaagatttagaaaaaggatgccattcttctataaaacttcagaggtatttaacaaacatggtaatgtgtttg
aatgttctattttggaagaaattccatatgagaaagatttgattattgaacgagaaaatttttggattaaagagcttaattctaaaatt
aatggatacaatattgctgatgcaacgtttggtgatacgtgttctacgcatccattaaaagaagaaattattaagaaacgttctgaaac
ttttaaagctaagatgcttaaacttggacctgatggtcggaaagctctttacagtaaacccggaagtaaaaacgggcgttggaatccag
aaacccataagttttgtaagtgcggtgttcgcatacaaacttctgcttatacttgtagtaaatgcagaaatggtggttctggtggtacc
ggaggtagcatggataaaaagtattctattggtttagacatcggcactaattccgttggatgggctgtcataaccgatgaatacaaagt
accttcaaagaaatttaaggtgttggggaacacagaccgtcattcgattaaaaagaatcttatcggtgccctcctattcgatagtggcg
aaacggcagaggcgactcgcctgaaacgaaccgctcggagaaggtatacacgtcgcaagaaccgaatatgttacttacaagaaattttt
agcaatgagatggccaaagttgacgattctttctttcaccgtttggaagagtccttccttgtcgaagaggacaagaaacatgaacggca
ccccatctttggaaacatagtagatgaggtggcatatcatgaaaagtacccaacgatttatcacctcagaaaaaagctagttgactcaa
ctgataaagcggacctgaggttaatctacttggctcttgcccatatgataaagttccgtgggcactttctcattgagggtgatctaaat
ccggacaactcggatgtcgacaaactgttcatccagttagtacaaacctataatcagttgtttgaagagaaccctataaatgcaagtgg
cgtggatgcgaaggctattcttagcgcccgcctctctaaatcccgacggctagaaaacctgatcgcacaattacccggagagaagaaaa
atgggttgttcggtaaccttatagcgctctcactaggcctgacaccaaattttaagtcgaacttcgacttagctgaagatgccaaattg
cagcttagtaaggacacgtacgatgacgatctcgacaatctactggcacaaattggagatcagtatgcggacttatttttggctgccaa
aaaccttagcgatgcaatcctcctatctgacatactgagagttaatactgagattaccaaggcgccgttatccgcttcaatgatcaaaa
ggtacgatgaacatcaccaagacttgacacttctcaaggccctagtccgtcagcaactgcctgagaaatataaggaaatattctttgat
cagtcgaaaaacgggtacgcaggttatattgacggcggagcgagtcaagaggaattctacaagtttatcaaacccatattagagaagat
ggatgggacggaagagttgcttgtaaaactcaatcgcgaagatctactgcgaaagcagcggactttcgacaacggtagcattccacatc
aaatccacttaggcgaattgcatgctatacttagaaggcaggaggatttttatccgttcctcaaagacaatcgtgaaaagattgagaaa
atcctaacctttcgcataccttactatgtgggacccctggcccgagggaactctcggttcgcatggatgacaagaaagtccgaagaaac
gattactccctggaattttgaggaagttgtcgataaaggtgcgtcagctcaatcgttcatcgagaggatgaccaactttgacaagaatt
taccgaacgaaaaagtattgcctaagcacagtttactttacgagtatttcacagtgtacaatgaactcacgaaagttaagtatgtcact
gagggcatgcgtaaacccgcctttctaagcggagaacagaagaaagcaatagtagatctgttattcaagaccaaccgcaaagtgacagt
taagcaattgaaagaggactactttaagaaaattgaatgcttcgattctgtcgagatctccggggtagaagatcgatttaatgcgtcac
ttggtacgtatcatgacctcctaaagataattaaagataaggacttcctggataacgaagagaatgaagatatcttagaagatatagtg
ttgactcttaccctctttgaagatcgggaaatgattgaggaaagactaaaaacatacgctcacctgttcgacgataaggttatgaaaca
gttaaagaggcgtcgctatacgggctggggacgattgtcgcggaaacttatcaacgggataagagacaagcaaagtggtaaaactattc
tcgattttctaaagagcgacggcttcgccaataggaactttatgcagctgatccatgatgactctttaaccttcaaagaggatatacaa
aaggcacaggtttccggacaaggggactcattgcacgaacatattgcgaatcttgctggttcgccagccatcaaaaagggcatactcca
gacagtcaaagtagtggatgagctagttaaggtcatgggacgtcacaaaccggaaaacattgtaatcgagatggcacgcgaaaatcaaa
cgactcagaaggggcaaaaaaacagtcgagagcggatgaagagaatagaagagggtattaaagaactgggcagccagatcttaaaggag
catcctgtggaaaatacccaattgcagaacgagaaactttacctctattacctacaaaatggaagggacatgtatgttgatcaggaact
ggacataaaccgtttatctgattacgacgtcgatcacattgtaccccaatcctttttgaaggacgattcaatcgacaataaagtgctta
cacgctcggataagaaccgagggaaaagtgacaatgttccaagcgaggaagtcgtaaagaaaatgaagaactattggcggcagctccta
aatgcgaaactgataacgcaaagaaagttcgataacttaactaaagctgagaggggtggcttgtctgaacttgacaaggccggatttat
taaacgtcagctcgtggaaacccgccaaatcacaaagcatgttgcacagatactagattcccgaatgaatacgaaatacgacgagaacg
ataagctgattcgggaagtcaaagtaatcactttaaagtcaaaattggtgtcggacttcagaaaggattttcaattctataaagttagg
gagataaataactaccaccatgcgcacgacgcttatcttaatgccgtcgtagggaccgcactcattaagaaatacccgaagctagaaag
tgagtttgtgtatggtgattacaaagtttatgacgtccgtaagatgatcgcgaaaagcgaacaggagataggcaaggctacagccaaat
acttcttttattctaacattatgaatttctttaagacggaaatcactctggcaaacggagagatacgcaaacgacctttaattgaaacc
aatggggagacaggtgaaatcgtatgggataagggccgggacttcgcgacggtgagaaaagttttgtccatgccccaagtcaacatagt
aaagaaaactgaggtgcagaccggagggttttcaaaggaatcgattcttccaaaaaggaatagtgataagctcatcgctcgtaaaaagg
actgggacccgaaaaagtacggtggcttcgatagccctacagttgcctattctgtcctagtagtggcaaaagttgagaagggaaaatcc
aagaaactgaagtcagtcaaagaattattggggataacgattatggagcgctcgtatttgaaaagaaccccatcgacttccttgaggcg
aaaggttacaaggaagtaaaaaaggatctcataattaaactaccaaagtatagtctgtttgagttagaaaatggccgaaaacggatgtt
ggctagcgccggagagcttcaaaaggggaacgaactcgcactaccgtctaaatacgtgaatttcctgtatttagcgtcccattacgaga
agttgaaaggttcacctgaagataacgaacagaagcaactttttgttgagcagcacaaacattatctcgacgaaatcatagagcaaatt
tcggaattcagtaagagagtcatcctagctgatgccaatctggacaaagtattaagcgcatacaacaagcacagggataaacccatacg
tgagcaggcggaaaatattatccatttgtttactcttaccaacctcggcgctccagccgcattcaagtattttgacacaacgatagatc
gcaaacgatacacttctaccaaggaggtgctagacgcgacactgattcaccaatccatcacgggattatatgaaactcggatagatttg
tcacagcttgggggtgacggatcccatcatcaccaccaccattgagcggccgcataatgcttaagtcgaacagaaagtaatcgtattgt
acacggccgcataatcgaaattccctatcagtgatagagattgacatccctatcagtgatagagatactgagcacgggagacccatgcc
atagcgttgttcggaatatgaatttttgaacagattcaccaacacctagtggtctcgttttagagctagaaatagcaagttaaaataag
gctagtccgttatcaacttgaaaaagtggcaccgagtcggtgctccgctgagcaataactagcataaccccttggggcctctaaacggg
tcttgaggggttttttggcgagcatcacgtgctataaaaataattataatttaaattttttaatataaatatataaattaaaaatagaa
agtaaaaaaagaaattaaagaaaaaatagtttttgttttccgaagatgtaaaagactctagggggatcgccaacaaatactacctttta
ccttgctcttcctgctctcaggtattaatgccgaattgtttcatcttgtctgtgtagaagaccacacacgaaaatcctgtgattttaca
ttttacttatcgttaatcgaatgtatatctatttaatctgatttcttgtctaataaatatatatgtaaagtacgctttttgttgaaatt
ttttaaacctttgtttatttttttttcttcattccgtaactcttctaccttctttatttactttctaaaatccaaatacaaaacataaa
aataaataaacacagagtaaattcccaaattattccatcattaaaagatacgaggcgcgtgtaagttacaggcaagcgatcctagtaca
ctctatatttttttatgcctcggtaatgattttcattttttttttccacctagcggatgactattttttttcttagcgattggcattat
cacataatgaattatacattatataaagtaatgtgatttcttcgaagaatatactaaaaaatgagcaggcaagataaacgaaggcaaag
atgacagagcagaaagccctagtaaagcgtattacaaatgaaaccaagattcagattgcgatctattaaagggtggtcccctagcgata
gagcactcgatcttcccagaaaaagaggcagaagcagtagcagaacaggccacacaatcgcaagtgattaacgtccacacaggtatagg
gtttctggaccatatgatacatgctctggccaagcattccggctggtcgctaatcgttgagtgcattggtgacttacacatagacgacc
atcacaccactgaagactgcgggattgctctcggtcaagatttaaagaggccctaggggccgtgcgtggagtaaaaaggtttggatcag
gatttgcgcctttggatgaggcactttccagagcggtggtagatctttcgaacaggccgtacgcagttgtcgaacttggtttgcaaagg
gagaaagtaggagatctctcttgcgagatgatcccgcattttcttgaaagctttgcagaggctagcagaattaccctccacgttgattg
tctgcgaggcaagaatgatcatcaccgtagtgagagtgcgttcaaggctcttgcggttgccataagagaagccacctcgcccaatggta
ccaacgatgttccctccaccaaaggtgttcttatgtagttttacacaggagtctggacttgactgaaacctcaggcatttgagaagcac
acggtcacactgcttccggtagtcaataaaccggtaaaccagcaatagacataagcggctatttaacgaccctgccctgaaccgacgac
cgggtcgaatttgctttcgaatttctgccattcatccgcttattatc

TABLE 5
Summary of sgRNA cloning

| Target | 1 | 2 | 3 | 4 | 5 | 6 | 7 | 8 |
|---|---|---|---|---|---|---|---|---|
| RplC | Y | 5 | 2 | 2 | 2 | 3 | 2 | insertion |
| YtfN | ? | 3 | 3 | 1 | 1 | 1 | 1 | NS |
| YghJ | N | 3 | 3 | 1 | 1 | 1 | 1 | correct |
| MrcB | ? | 3 | 3 | 1 | 1 | 1 | 1 | NS |
| AegA | N | 3 | 3 | 1 | 1 | 1 | 1 | correct |
| GltJ | N | 3 | 3 | 1 | 1 | 1 | 1 | correct |
| OmpS | ? | 3 | 3 | 1 | 1 | 1 | 1 | NS |
| MviM | ? | 3 | 3 | 1 | 1 | 1 | 1 | NS |
| STM1005 | N | 2 | 2 | 1 | 1 | 1 | 1 | correct |
| STM4261 | N | 2 | 1 | 1 | 1 | 1 | 1 | correct |
| FabB | Y | 12 | 12 | 2 | 2 | 1 | 1 | correct |
| MurE | Y | 5 | 5 | 1 | 1 | 1 | 1 | correct |
| Tsf | Y | 10 | 2 | 1 | 1 | 1 | 1 | correct |
| FtsW | Y | 20 | 17 | 9 | 8 | 13 | 8 | insertion |
| RpoB | Y | 32 | 11 | 8 | 1 | 5 | 3 | insertion |
| PolA | Y | 5 | 5 | 1 | 1 | 1 | 1 | correct |
| IcdA | Y | 4 | 4 | 1 | 1 | 1 | 1 | correct |
| NarY | Y | 4 | 4 | 1 | 1 | 1 | 1 | correct |
| ClpX | Y | 4 | 4 | 1 | 1 | 1 | 1 | insertion |
| ArgS | Y | 29 | 15 | 4 | 1 | 9 | 3 | insertion |
| | | | | | | | | ×2 |
| TrmD | Y | 19 | 13 | 6 | 3 | 10 | 5 | insertion |
| PrfA | Y | 14 | 5 | 1 | 1 | 1 | 1 | correct |
| LepA | Y | 19 | 17 | 6 | 6 | 16 | 6 | insertion |
| PolA.1 | Y | 3 | 3 | 1 | 1 | 1 | 1 | NS |
| PolA.2 | Y | 3 | 3 | 1 | 1 | 1 | 1 | correct |
| PolA.3 | Y | 3 | 3 | 1 | 1 | 1 | 1 | correct |
| PolA.4 | Y | 23 | 17 | 2 | 1 | 4 | 1 | NS |
| PolA.5 | Y | 15 | 5 | 1 | 1 | 1 | 1 | NS |
| PolA.6 | Y | 3 | 3 | 1 | 1 | 1 | 1 | NS |
| PolA.7 | Y | 3 | 3 | 1 | 1 | 1 | 1 | NS |
| PolA.8 | Y | 3 | 3 | 1 | 1 | 1 | 1 | NS |
| PolA.9 | Y | 3 | 3 | 1 | 1 | 1 | 1 | correct |
| PolA.10 | Y | 3 | 3 | 1 | 1 | 1 | 1 | NS |
| PolA.11 | Y | 3 | 3 | 3 | 2 | 1 | 1 | correct |
| PolA.12 | Y | 3 | 3 | 1 | 1 | 1 | 1 | NS |
| PolA.13 | Y | 3 | 3 | 1 | 1 | 1 | 1 | NS |
| PolA.14 | Y | 3 | 3 | 1 | 1 | 1 | 1 | NS |
| PolA.15 | Y | 3 | 3 | 1 | 1 | 1 | 1 | correct |
| PolA.16 | Y | 3 | 3 | 1 | 1 | 1 | 1 | correct |
| PolA.18 | Y | 3 | 3 | 1 | 1 | 1 | 1 | NS |
| KatG.1 | N | 3 | 3 | 1 | 1 | 1 | 1 | correct |
| KatG.2 | N | 3 | 3 | 1 | 1 | 1 | 1 | NS |
| KatG.3 | N | 3 | 3 | 1 | 1 | 1 | 1 | correct |
| KatG.4 | N | 3 | 3 | 1 | 1 | 1 | 1 | correct |
| KatG.5 | N | 3 | 3 | 1 | 1 | 1 | 1 | correct |
| KatG.6 | N | 3 | 2 | 1 | 1 | 1 | 1 | correct |
| KatG.7 | N | 3 | 3 | 1 | 1 | 1 | 1 | correct |
| KatG.8 | N | 3 | 3 | 1 | 1 | 1 | 1 | correct |
| KatG.9 | N | 3 | 3 | 1 | 1 | 1 | 1 | NS |
| KatG.10 | N | 3 | 3 | 1 | 1 | 1 | 1 | NS |
| KatG.11 | N | 3 | 3 | 1 | 1 | 1 | 1 | correct |
| KatG.12 | N | 3 | 3 | 1 | 1 | 1 | 1 | NS |
| KatG.13 | N | 3 | 3 | 1 | 1 | 1 | 1 | NS |
| KatG.14 | N | 3 | 2 | 1 | 1 | 1 | 1 | NS |
| KatG.15 | N | 3 | 3 | 1 | 1 | 1 | 1 | NS |
| KatG.16 | N | 3 | 3 | 1 | 1 | 1 | 1 | correct |
| KatG.17 | N | 3 | 3 | 1 | 1 | 1 | 1 | NS |
| KatG.18 | N | 3 | 3 | 1 | 1 | 1 | 1 | correct |
| fabB.1 | Y | 4 | 2 | 1 | 0 | 2 | 1 | NS |
| fabB.2 | Y | 3 | 0 | 1 | 0 | 0 | 0 | NS |
| fabB.3 | Y | 3 | 3 | 0 | 0 | 1 | 0 | NS |
| fabB.4 | Y | 3 | 1 | 0 | 0 | 1 | 0 | NS |
| fabB.5 | Y | 3 | 3 | 0 | 0 | 1 | 0 | NS |
| fabB.6 | Y | 3 | 3 | 1 | 1 | 2 | 2 | NS |
| fabB.7 | Y | 4 | 1 | 0 | 0 | 1 | 0 | NS |
| fabB.8 | Y | 3 | 3 | 0 | 0 | 3 | 0 | NS |
| fabB.9 | Y | 3 | 3 | 1 | 1 | 2 | 1 | NS |
| fabB.10 | Y | 3 | 3 | 1 | 1 | 2 | 2 | NS |
| fabB.11 | Y | 3 | 3 | 1 | 1 | 2 | 2 | NS |
| fabB.12 | Y | 3 | 0 | 2 | 0 | 0 | 0 | NS |
| fabB.13 | Y | 4 | 0 | 2 | 0 | 0 | 0 | NS |
| fabB.14 | Y | 3 | 2 | 1 | 0 | 2 | 1 | NS |
| fabB.15 | Y | 3 | 0 | 1 | 0 | 0 | 0 | NS |
| fabB.16 | Y | 3 | 3 | 0 | 0 | 2 | 0 | NS |
| fabB.17 | Y | 3 | 3 | 0 | 0 | 3 | 0 | NS |
| fabB.18 | Y | 4 | 2 | 0 | 0 | 2 | 0 | NS |
| fabB.19 | Y | 3 | 3 | 2 | 0 | 2 | 2 | NS |
| fabB.20 | Y | 3 | 3 | 1 | 1 | 2 | 2 | NS |

Column label 1: Gene function.
Column label 2: Number of colonies screened.
Column label 3: Number of positive PCR Screens.
Column label 4: Number send for sequencing.
Column label 5: Number with correct gRNA sequence.
Column label 6: Number of clones digested.
Column label 7: Number of correct digests.
Column label 8: Full plasmid sequencing results.

### REFERENCES

1. Whipps, J. M. (2001) Microbial interactions and biocontrol in the rhizosphere. Journal of experimental Botany, 52(suppl 1), 487-511.
2. O'Toole, P. W. and Jeffery, I. B. (2015) Gut microbiota and aging. Science, 350(6265), 1214-1215.
3. Gerard, P. (2016) Gut microbiota and obesity. Cellular and molecular life sciences, 73(1), 147-162.
4. Sun, J. and Kato, I. (2016) Gut microbiota, inflammation and colorectal cancer. Genes & diseases, 3(2), 130-143.
5. Yu, Y., Champer, J., Beynet, D., Kim, J., and Friedman, A. J. (2015) The role of the cutaneous microbiome in skin cancer: lessons learned from the gut. Journal of drugs in dermatology: JDD, 14(5), 461-465.
6. Chatain-Ly, M. H. (2014) The factors affecting effectiveness of treatment in phages therapy. Frontiers in microbiology, 5, 51.
7. Reid, G., Younes, J. A., Van der Mei, H. C., Gloor, G. B., Knight, R., and Busscher, H. J. (2011) Microbiota restoration: natural and supplemented recovery of human microbial communities. Nature Reviews Microbiology, 9(1), 27.
8. Khanna, S., Vazquez-Baeza, Y., Gonz alez, A., Weiss, S., Schmidt, B., Mu˜niz-Pedrogo, D. A., Rainey, J. F., Kammer, P., Nelson, H., Sadowsky, M., et al. (2017) Changes in microbial ecology after fecal microbiota transplantation for recurrent C. difficile infection affected by underlying inflammatory bowel disease. Microbiome, 5(1), 55.
9. Petrof, E. O., Gloor, G. B., Vanner, S. J., Weese, S. J., Carter, D., Daigneault, M. C., Brown, E. M., Schroeter, K., and Allen-Vercoe, E. (2013) Stool substitute transplant therapy for the eradication of Clostridium difficile infection: ‘RePOOPulating’ the gut. Microbiome, 1(1), 3.
10. Martz, S. L., Guzman-Rodriguez, M., He, S.-M., Noordhof, C., Hurlbut, D. J., Gloor, G. B., Carlucci, C., Weese, S., Allen-Vercoe, E., Sun, J., et al. (2017) A human gut ecosystem protects against C. difficile disease by targeting TcdA. Journal of gastroenterology, 52(4), 452-465.
11. Jinek, M., Chylinski, K., Fonfara, I., Hauer, M., Doudna, J. A., and Charpentier, E. (2012) A programmable dual-RNA-guided DNA endonuclease in adaptive bacterial immunity. Science, p. 1225829.
12. Citorik, R. J., Mimee, M., and Lu, T. K. (2014) Sequence-specific antimicrobials using efficiently delivered RNA-guided nucleases. Nature biotechnology, 32(11), 1141.
13. Bikard, D., Euler, C. W., Jiang, W., Nussenzweig, P. M., Goldberg, G. W., Duportet, X., Fischetti, V. A., and Marraffini, L. A. (2014) Exploiting CRISPR-Cas nucleases to produce sequence-specific antimicrobials. Nature biotechnology, 32(11), 1146.
14. Gomaa, A. A., Klumpe, H. E., Luo, M. L., Selle, K., Barrangou, R., and Beisel, C. L. (2014) Programmable removal of bacterial strains by use of genome-targeting CRISPR-Cas systems. MBio, 5(1), e00928-13.
15. Wolfs, J. M., Hamilton, T. A., Lant, J. T., Laforet, M., Zhang, J., Salemi, L. M., Gloor, G. B., Schild-Poulter, C., and Edgell, D. R. (2016) Biasing genome-editing events toward precise length deletions with an RNA-guided TevCas9 dual nuclease. Proceedings of the National Academy of Sciences, 113(52), 14988-14993.
16. Gibson, D. G. (2009) Synthesis of DNA fragments in yeast by one-step assembly of overlapping oligonucleotides. Nucleic acids research, 37(20), 6984-6990.
17. Noskov, V. N., Karas, B. J., Young, L., Chuang, R.-Y., Gibson, D. G., Lin, Y.-C., Stam, J., Yonemoto, I. T., Suzuki, Y., Andrews-Pfannkoch, C., et al. (2012) Assembly of large, high G+C bacterial DNA fragments in yeast. ACS synthetic biology, 1(7), 267-273.
18. Strand, T. A., Lale, R., Degnes, K. F., Lando, M., and Valla, S. (2014) A new and improved host-independent plasmid system for RK2-based conjugal transfer. PloS one, 9(3), e90372.

Thus, it should be understood that although the present invention has been specifically disclosed by preferred embodiments and optional features, modification, improvement and variation of the inventions embodied therein herein disclosed may be resorted to by those skilled in the art, and that such modifications, improvements and variations are considered to be within the scope of this invention. The materials, methods, and examples provided here are representative of preferred embodiments, are exemplary, and are not intended as limitations on the scope of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

It is to be understood that while the invention has been described in conjunction with the above embodiments, that the foregoing description and examples are intended to illustrate and not limit the scope of the invention. Other aspects, advantages and modifications within the scope of the invention will be apparent to those skilled in the art to which the invention pertains.

1. An isolated or recombinant cis-conjugative plasmid comprising:
(a) conjugation genes;
(b) a gene or a combination of genes capable of being expressed in a target bacteria within a microbiome or biofilm and that modulates the target bacteria in the microbiome or biofilm; and
(c) a single or multiple single-guide RNAs corresponding to a single or multiple target sites of the target bacteria.

2. The isolated or recombinant cis-conjugative plasmid of claim 1, wherein the gene that modulates the target bacteria is a coding region for a dual nuclease gene.

3. The isolated or recombinant cis-conjugative plasmid of claim 1, wherein the gene that modulates the target bacteria is a coding region for TevCas9 nuclease gene.

4. The isolated or recombinant cis-conjugative plasmid of claim 1, wherein the gene that modulates the target bacteria is a coding region for a site-specific DNA endonuclease

5. The isolated or recombinant cis-conjugative plasmid of claim 1, wherein the gene that modulates the target bacteria is a coding region for a bacterial toxin, wherein the bacterial toxin includes DNA gyrase inhibitors or topoisomerase inhibitors.

6. The isolated or recombinant cis-conjugative plasmid of claim 1, wherein the gene that modulates the target bacteria is a coding region for a gene or genes for biosynthetic or biodegradative pathways.

7. The isolated or recombinant cis-conjugative plasmid of claim 1, wherein the gene that modulates the target bacteria is a coding region for regulatory sequence including small RNA molecules or transcription factors.

8. (canceled)

9. (canceled)

10. A method for inhibiting, preventing or treating an infection caused by an organism (“target organism”) that can accept by conjugation and express a conjugative plasmid in a subject, comprising administering to the subject an effective amount of a cis-conjugative plasmid comprising (a) conjugation genes, (b) a gene or a combination of genes capable of being expressed in the target organism and that modulates the target organism that causes the infection, and (c) a single or multiple single-guide RNAs corresponding to a single or multiple target sites of the target bacteria, thereby inhibiting, preventing or treating the infection.

11. (canceled)

12. The method of claim 8, wherein the gene that modulates the target organism is a coding region for a dual nuclease gene.

13. The method of claim 8, wherein the gene that modulates the target organism is a coding region for TevCas9 nuclease gene.

14. The method of claim 8, wherein the gene that modulates the target organism is a coding region for a site-specific DNA endonuclease.

15. The method of claim 8, wherein the gene that modulates the target organism is a coding region for a bacterial toxin, wherein the bacterial toxin includes DNA gyrase inhibitors or topoisomerase inhibitors.

16. The method of claim 8, wherein the gene that modulates the target organism is a coding region for a gene or genes for biosynthetic or biodegradative pathways.

17. The method of claim 8, wherein the gene that modulates the target organism is a coding region for regulatory sequence including small RNA molecules or transcription factors.

18. (canceled)

19. The method of claim 8, wherein the target organism is a bacterium.

20. The method of claim 8, wherein the gene that modulates the target organism in the cis-conjugative plasmid modulates only the target organism.

21-30. (canceled)

31. A method of detecting the presence of a bacteria of interest in a microbiome, the method comprising contacting the microbiome with a cis-conjugative plasmid comprising (a) conjugation genes, (b) a detectable gene specific for the bacteria of interest and (c) a single or multiple single-guide RNAs corresponding to a single or multiple target sites of the bacteria of interest.

32. (canceled)

33. The method of claim 31, wherein the detectable gene expresses a detectable protein when the detectable gene is activated by an activator when the activator is in operative proximity to the detectable gene.

34. The method of claim 33, wherein the activator is a transcriptional activation domain.

35. The method of claim 31, wherein the detectable gene is a transposon for transposon-based tagging.

36-39. (canceled)

40. The method of claim 31, wherein the microbiome is a site of an infection and the bacteria of interest is bacteria that causes the infection, and wherein the detectable gene is specific for the bacteria that causes the infection.

41. The isolated or recombinant cis-conjugative plasmid of claim 1, wherein the cis-conjugative plasmid comprises SEQ ID NO:66 or an isolated or recombinant nucleic acid sequence having at least 80% sequence identity to SEQ ID NO:66.
